(19) United States
(12) Patent Application Publication
(10) Pub. No.: US 2010/0166789 A1
(43) Pub. Date: Jul. 1, 2010

(54) PROTEINS FOR USE IN DIAGNOSING AND TREATING INFECTION AND DISEASE

(75) Inventors: Haig Keledjian, San Marino, CA (US); Michael Agadjanyan, Huntington Beach, CA (US); Martha Karen Newell, Colorado Springs, CO (US); Evan Newell, Menlo Park, CA (US)

Correspondence Address: WOLF GREENFIELD & SACKS, P.C., 600 ATLANTIC AVENUE, BOSTON, MA, 02210-2206, US

(73) Assignees: The Regents of the University of Colorado, Denver, CO (US); Viral Genetics, Inc., San Marino, CA (US)

(21) Appl. No.: 12/508,532

(22) Filed: Jul. 23, 2009

Related U.S. Application Data
(60) Provisional application No. 61/135,922, filed on Jul. 25, 2008.

(51) Int. Cl. A61K 39/00; A61K 38/08; A61K 38/10; A61K 38/16; G01N 33/53; C07K 7/06; C07K 7/08; C07K 14/00; A61P 37/02; A61P 31/18

(52) U.S. Cl. 424/185.1; 514/14; 514/13; 514/15; 514/12; 435/7.21; 530/327; 530/326; 530/325; 530/324; 530/328

(57) ABSTRACT

The present invention describes compositions of thymus derived peptides and uses therefore in diagnostic methods and for the treatment of diseases associated with reduced T helper cell counts, diseases such as infection, e.g., HIV infection and other viral infections, parasitic, and bacterial infection, AIDS, ARC, multiple sclerosis, chronic fatigue syndrome, rheumatoid arthritis, Alzheimer's disease, asthma, allergy, dermatitis, type 1 diabetes mellitus, colitis, inflammatory bowel disease/irritable bowel syndrome, Crohn's disease, Psoriasis, Chronic obstructive pulmonary disease, Systemic lupus erythematosus, transplant rejection and cancer.

### RELATED APPLICATIONS

This application claims priority under 35 U.S.C. §119 from U.S. provisional application Ser. No. 61/135,922, filed Jul. 25, 2008 the contents of which are incorporated herein in their entirety.

### FIELD OF THE INVENTION

This invention relates to the areas of immunology and virology and specifically relates to thymus derived peptides, which are useful as diagnostics and therapeutics for infectious disease such as viral infection e.g. human immunodeficiency virus (HIV) infection and related diseases such as acquired immunodeficiency syndrome (AIDS) and AIDS-related complex (ARC), as well as other viral, parasitic, bacterial infections, diseases associated with a decrease in T cell count, autoimmune disease, graft rejection, Alzheimer's disease, allergic disease, and cancer.

### BACKGROUND OF INVENTION

Bone marrow produces cells which are destined to become immune cells. These cells become lymphocytes or phagocytes. Lymphocytes are small white blood cells that bear the major responsibility for carrying out the activities of the immune system. The two major classes of lymphocytes are B cells and T cells. B cells mature in the bone (thus the term “B cells”) marrow. T cells migrate to the thymus (thus the term “T cells”) where they multiply and mature into cells capable of immune response. Upon exiting the bone marrow and thymus, both B and T cells travel widely and continuously throughout the body.

There are two types of T cells, regulatory and cytotoxic T cells, which contribute to the immune defenses in at least two major ways. Chief among the T cells are “helper/inducer” cells. Identifiable by the T4 cell marker, helper T cells are essential for activating B cells and other T cells as well as natural killer cells and macrophages. Cytotoxic T cells are killer cells which, for example, directly attack and rid the body of cells that have been infected by viruses or transformed by cancer.

Important phagocytes are monocytes and macrophages. Monocytes circulate in the blood, then migrate into tissues where they develop into macrophages (“big eaters”). Macrophages are found throughout the body tissues and are versatile cells that play many roles. As scavengers, they rid the body of worn-out cells and other debris. Foremost among cells that present antigen to T cells, having first digested and processed it, macrophages playa crucial role in initiating the immune response. As secretory cells, monocytes and macrophages are vital to the regulation of immune responses. They also carry receptors for lymphokines that allow them to be “activated” to pursue microbes and tumor cells.

Some diseases, such as Acquired Immunodeficiency Syndrome (AIDS,) are caused by a virus, in the case of AIDS, the human immunodeficiency virus (HIV). Such viruses destroy helper T cells and, again using AIDS as an example, is harbored in macrophages and monocytes. Entry of HIV-1 into helper T cells involves the primary receptor CD4 and co-receptors CCR5 and CXCR4. The first step in cell entry occurs when the HIV-1 glycoprotein gp120 binds to the CD4 receptors on target cells. The next step is an interaction between the HIV-1 envelope protein and the co-receptor CCR5. Once gp120 interacts with receptor and co-receptor, the HIV-1 envelope protein gp41 undergoes a conformational change and literally brings the viral membrane into close proximity with the cell membrane. Fusion of two lipid bilayers then occurs, allowing intracellular entry of the viral contents (see, for example, Nature (1997) 387:426-430).

When HIV infects a human patient, it incorporates itself into the deoxyribonucleic acid (DNA) of the immune cells and for a variable period of between 3 months to years, the patient may not exhibit any-immunodeficiency symptoms and sometimes does not produce a detectable level of antibodies against AIDS. Since an initial HIV infection may not immediately lead to detectable clinical disease symptoms or a detectable level of antibodies, the term “HIV infection” as used herein encompasses both the infection and any disease resulting therefrom, the latter being termed “HIV-related diseases”. Examples of HIV-related diseases are AIDS and ARC. After the above incubation period, the HIV multiplies within the infected cell and eventually bursts the host cells which release the newly formed viruses. Since the host cells are destroyed in the process, the patient's immune system is impaired and the host is susceptible to opportunistic diseases that a human with intact immune system is not susceptible to. In human, generally the AIDS virus will multiply and the human will eventually die from severe immunodeficiency. Interestingly, only humans suffer from AIDS. When a non-human mammal, such as a rabbit, mouse, rat or cow, is injected with HIV, the animal may temporarily have some T cells destroyed. However, 14 to 21 days post-infection, the animal would mount an antibody attack and does not succumb to AIDS.

Currently, despite enormous efforts there is no cure for AIDS and the available therapeutic treatments have limited, and in some cases negligible, results.

Accurately diagnosing AIDS at an earlier stage of the disease has also been the focal point of research efforts. Currently, the commercially available diagnostic tests are generally directed to detecting the patient's antibodies against HIV. But antibody production against the virus generally does not occur until about 14 to 21 days after the time the patient is infected with AIDS. Therefore, if a patient is tested before antibody to production has begun and is quantitatable; the tests will produce a false negative result. On the other hand, some of these tests may also give false positive results due to non-specific binding of the antibodies. Another means for detecting the viral infection is through nucleic acid hybridization.

Unless otherwise noted, the following is based on Stein et al. (1992) Infect. Diseases, 165: 352. The surrogate marker that most closely correlates with the stage of HIV infection is the CD4+ or T helper, cell count. HIV-1 envelope glycoprotein, gp120, specifically binds to the CD4 receptor that is expressed in greatest concentration in a subset of T lymphocytes and in lower amounts on monocytes and macrophages. Cells expressing CD4 receptors are termed the “helper/inducer” subset, reflecting their role as both helper cells for B cell responses for antigens expressed on cells bearing human leukocyte antigen (HLA) class II receptors and inducer cells that cause T cells to suppress immune responses. The selective loss of CD4+ cells results in numerous immune defects associated with susceptibility to the opportunistic infections that are the hallmark of AIDS.

The HIV core antigen p24 can be detected before the appearance of HIV antibodies. After the appearance of HIV antibodies by the screening enzyme-linked immunosorbent assay (ELISA), p24 aritigenemia generally becomes undetectable, though it can occasionally persist and often will recur later in the disease. HIV-I titers found in plasma and peripheral blood mononuclear cell cultures also fall rapidly as specific antibodies are detectable, suggesting at least a transiently effective host immune response. Markers of immune stimulation include P2-microglobulin.

In patients followed from the time of seroconversion, CD4+ cell decline has been correlated with progression to AIDS. Serum levels of P2-microglobulin and detection of p24 antigen in blood were also both independently correlated with rates of progression. Combined with CD⁴ cell counts, use of p2-microglobulin and p24 antigen increased prognostic accuracy for progression to AIDS compared with CD4+ cell count alone.

Increased CD8+ cell counts were found to be somewhat predictive of subsequent development of AIDS. To better correlate clinical end points, such as survival and progression to AIDS, with surrogate markers of antiviral therapy effects, analysis of additional markers such as neopterin and P2-microglobulin, among others, have been combined with the CD4 cell count and p24 antigen.

In a limited study (Jacobson (1991) BNJ, 302:73) of patients with AIDS and to ARC who tolerated an anti-AIDS drug, zidovudine, and who survived for 12 weeks, the following was found.

After controlling for three factors (age, diagnosis of AIDS at baseline, log of the baseline serum neopterin concentration), the log of the CD4+ cell count at 8-12 weeks, but not the change over time, best predicted subsequent survival. A decrease in P2 microglobulin concentration at 8-12 weeks significantly predicted survival and, combined with the log of the CD4+ cell count, provided the best predictive model. Decreases in p24 antigenemia, serum neopterin concentrations, and the Karhofsky performance status (a measure of function in routine activities) did not significantly correlate with survival on therapy.

Stein et al. (1992 Infect. Diseases 165:352), conclude that changes in CD4+ cell counts and other surrogate markers may be increasingly used as the sole end point for investigations of antiretroviral activity, of a drug or therapy, in patients with early HIV infection.

Other diseases, such as type 1 diabetes mellitus, colitis and Crohn's disease, are not currently known to be caused by viral infection. But these diseases are also associated with a decrease in the number of helper T (T_{H}) cells.

### SUMMARY OF INVENTION

The invention is based at least in part on the discovery that peptides of thymus derived extracts are useful in the treatment of disorders such as viral and bacterial infection, autoimmune disease, tissue graft rejection and cancer. The invention is also based on the discovery of the specific sequence of such peptides.

In some aspects the invention is a composition of a thymus derived peptide and a pharmaceutically acceptable carrier. In some embodiments the composition is free of cystatin A protein and a histone protein. In other embodiments the composition includes cystatin A protein and/or a histone protein. The thymus derived peptide may be a peptide of any of SEQ ID NO. 1-SEQ ID NO. 265. In some embodiments the composition includes 1 thymus derived peptide. In other embodiments the composition includes 2-100 thymus derived peptides.

In some embodiments the thymus derived peptide is synthetic. In other embodiments the thymus derived peptide is derived from natural sources.

The composition may also include other compounds. For instance the composition may include an adjuvant, such as, for instance, aluminum hydroxide or aluminum phosphate, calcium phosphate, mono phosphoryl lipid A, ISCOMs with Quil-A, or Syntex adjuvant formulations (SAFs) containing the threonyl derivative or muramyl dipeptide. Other compounds in the composition may be, for instance, an anti-viral agent, an anti-bacterial agent, an anti-cancer agent, or an anti-HIV agent.

In some embodiments the composition has a binding affinity for gp120 of at least 5000 RD. In other embodiments the composition has a binding affinity for gp41 of at least 5000 RD or for CD4 of at least 5000 RU.

The composition may include, in some embodiments, thymus derived peptide complexed to at least one protein selected from the group consisting of CD4, gp120 and gp41.

In other embodiments the composition is formulated for oral, intranasal, or pulmonary administration.

According to other aspects of the invention a method for treating HIV infection is provided. The method involves administering to a human infected with HIV or at risk of HIV infection a composition comprising a thymus derived peptide and a pharmaceutically acceptable carrier. In some embodiments the composition does not include every peptide of a thymus nuclear protein extract. In other embodiments the composition is not a thymus nuclear protein extract. In some embodiments the composition is free of cystatin A protein or a histone protein. In other embodiments the composition includes cystatin A protein and/or a histone protein. In some embodiments the thymus derived peptide is complexed to at least one protein selected from the group consisting of CD4, gp120 and gp41.

The composition may be administered on any therapeutically effective schedule or dosage. In some embodiments the administration occurs over a period of eight weeks. In other embodiments the administration is bi-weekly. The bi-weekly administration is optionally on consecutive days. In other embodiments the administration is at least one of oral, parenteral, subcutaneous, intravenous, intranasal, pulmonary, intramuscular and mucosal administration.

The composition in some embodiments has a binding affinity for gp120 of at least 5000 RD. In other embodiments the composition has a binding affinity for gp41 of at least 5000 RD or for CD4 of at least 5000 RU.

The composition may be administered in conjunction with other agents, such as an adjuvant or an anti-HIV agent. In some embodiments the adjuvant is aluminum hydroxide or aluminum phosphate. In other embodiments the adjuvant is calcium phosphate, aluminum salt adjuvants, such as aluminum phosphate or aluminum hydroxide, calcium phosphate nanoparticles (BioSante Pharmaceuticals, Inc.), ZADAXIN™, nucleotides ppGpp and pppGpp, killed Bordetella pertussis or its components, Corenybacterium derived P40 component, killed cholera toxin or its parts or killed mycobacteria or its parts.

A method for diagnosing HIV infection is provided in other aspects. The method involves collecting a sample from a subject; mixing the sample with a thymus derived peptide; and identifying a complex of the thymus derived peptide bound to CD4, gp120 or gp41, wherein the complex is indicative of HIV infection. The complex is identified in some embodiments by electrophoresis, chromatography, HPLC, or an immunological reaction. In other embodiments the sample is blood, serum or plasma.

A kit for detection of HIV is provided in other aspects of the invention. The kit includes a container housing a thymus derived peptide; a reagent for identifying at least one complex of said cystatin A protein and said at least one histone protein with CD4, gp120 or gp41; and instructions for identifying a complex that is indicative of HIV infection.

In another aspect the invention is a kit including a container housing a thymus derived peptide wherein the thymus derived peptide is a peptide of any of SEQ ID NO. 1-SEQ ID NO. 265; and instructions for identifying a complex that is indicative of HIV-I infection.

A method for treating a disease associated with a decrease in the number of T_{H} cells is provided according to other aspects of the invention. The method involves administering to a subject in need thereof a composition comprising a thymus derived peptide and a pharmaceutically acceptable carrier, in an effective amount to treat the disease. In some embodiments the composition does not include every peptide of a thymus nuclear protein extract. In other embodiments the composition is not a thymus nuclear protein extract. In some embodiments the composition is free of cystatin A protein or a histone protein. In other embodiments the composition includes cystatin A protein and/or a histone protein.

In some embodiments the disease is an autoimmune disease such as multiple sclerosis, rheumatoid arthritis, dermatitis, type 1 diabetes mellitus, colitis, inflammatory to bowel disease/irritable bowel syndrome, Crohn's disease, Psoriasis, and Systemic lupus erythematosus. In other embodiments the disease is chronic fatigue syndrome, Alzheimer's disease, or chronic obstructive pulmonary disease.

According to other aspects the invention is a method for treating cancer by administering to a subject having cancer a composition comprising a thymus derived peptide and a pharmaceutically acceptable carrier, in an effective amount to treat the cancer in the subject. In some embodiments the composition does not include every peptide of a thymus nuclear protein extract. In other embodiments the composition is not a thymus nuclear protein extract. In some embodiments the composition is free of cystatin A protein or a histone protein. In other embodiments the composition includes cystatin A protein and/or a histone protein.

In other aspects the invention is a method for treating a subject having a cell or tissue graft by administering to the subject in need thereof a composition comprising a thymus derived peptide and a pharmaceutically acceptable carrier, in an effective amount to inhibit cell or tissue graft rejection in the subject. In some embodiments the graft tissue or cell is heart, lung, kidney, skin, cornea, liver, neuronal tissue or cell, stem cell, including hematopoietic or embryonic stem cell. In some embodiments the composition does not include every peptide of a thymus nuclear protein extract. In other embodiments the composition is not a thymus nuclear protein extract. In some embodiments the composition is free of cystatin A protein or a histone protein. In other embodiments the composition includes cystatin A protein and/or a histone protein.

The current invention, therefore, discloses compositions, kits and methods for using thymus derived peptide. Methods for use include, for instance, methods for making diagnostics and therapeutics for HIV infection, AIDS and ARC and other diseases associated with a decrease in helper T cell numbers. Diseases associated with a decrease in the number of T_{H} cells are described for instance in Simpson et al. (2002) Clin Exp Allergy 32:37-42; Bottini et al. (2005) Intl Arch Allergy Immunol 138:328-333), such as multiple sclerosis (Nakajima et al. (2004) European Neurology 52:162-168), chronic fatigue syndrome, rheumatoid arthritis (Leader (1998) Ann Rheum Dis 57:328330, Alzheimer's disease, dermatitis (Feizy and Ghobadi, Dermatology Online Journal 12(3):3), type 1 diabetes mellitus (Feizy and Ghobadi, Dermatology Online Journal 12(3):3), colitis (Fort et al. (2001) J Immunol 166:2793-2800), inflammatory bowel disease/irritable bowel syndrome (Weinstock and Summers (2001) Currents Vol 2, Number 1; Fichtner-Feigl et al. (2005) J Clin Invest doi: 1 0.1172/JCI24792), Crohn's diseuse (Sato et al. (2005) Gut 54:1254-1262), Psoriasis (Simpson et al. (2002) Clin Exp Allergy 32:37-42), Chronic obstructive pulmonary disease (Bottini et al. (2005) Intl Arch Allergy Immunol 138:328-333), System lupus erythematosus, transplant rejection and cancer (Wu et al. (2005) Leukemia 19:268-274; Vujanovic et al. (2006) Cancer Gene Therapy 13:798-805).

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 TNP binds to HIV-1 envelope glycoproteins and human CD4 molecules. (A) 10% SDS-PAGE analysis of TNP following by Coomassie stain (Lane 1 Molecular-weight standards; Lane 2-TNP 80 μg/mL). Representative binding sensorgrams of TNP to human CD4 molecule (B), HIV-1 full-length gp41 (C) and gp120 (D) glycoproteins immobilized on a Biacore sensor chip (8 μg/mL, 1.6 μg/mL; 0.4 μg/mL).

FIG. 2 presents SDS-PAGE analysis of TNP proteins purified via binding to HIV-1 gp120 and CD4.

FIG. 3 presents representative binding activities of histone fraction HI, a heterogeneous mixture of all histone fractions, unfractionated whole histone and BSA to human CD4 and HIV-1 gp120.

FIG. 4 depicts CLIP displacement from the surface of Raji B cells lines in response to no treatment (4A and 4C) or treatment with MKN.5 (4B and 4D) for 4 (4A and 4B) and 24 hours (4C and 4D).

FIG. 5 depicts CLIP displacement from the surface of Daudi B cells lines in response to no treatment (5A and 5C) or treatment with MKN.5 (5B and 5D) for 4 (5A and 5B) and 24 hours (5C and 5D).

FIG. 6 depicts CLIP displacement from the surface of Raji (6B) or Daudi (6A) B cells lines in response to treatment with FRIMAVLAS for 24 hours.

FIG. 7 is a set of bar graphs depicting CLIP (7A), HLA DR, DP, DQ (7B) staining on the surface of Daudi cells in response to no treatment, or treatment with MKN.4 or MKN.6.

FIG. 8 depicts CLIP (y-axis) and HLA DR (x-axis) staining on the surface of B cells in response to no treatment, or treatment with MKN.4 or MKN.10.

FIG. 9 is a picture of a gel demonstrating binding of TNP extract to gp41. Lanes 1 and 2 were loaded with 7 μL TNP extract plus 6 μL gp41; lanes 3 and 4 with 7 μL TNP extract alone; and lane 5 was loaded with 6 μL gp41. Symbols indicate the position of the anode (−) and cathode (+).

FIG. 10 depicts the sonogram results demonstrating binding of bovine TNP to human CD4 molecules.

FIG. 11 is a graph depicting the binding of active component of TNP extract to CD4 molecules. The Biacore sensorgrams showing kinetics of the specific binding and subsequent dissociation of TNP's active component with immobilized CD4. Three sensorgrams correspond to different dilutions of one TNP sample received from Viral Genetics, Inc. The Arrow indicates the end of TNP injection and response at this time used for estimation of the sample's active component binding capacity at total concentration of sample.

FIG. 12 is a graph showing the percentage of patients responding to treatment with TNP extract in the South African study.

FIG. 13 is a graph showing the baseline CD4 and % Responders at day 150 and day 240. Patients who were on VGV-1 with lower CD4 counts (the red columns) were much more likely to have a good response than placebo patients (orange and blue columns). Patients on VGV-1 that were healthier (grey columns) also did not do as well as these sicker patients that received VGV-1.

### DETAILED DESCRIPTION

For clarity of disclosure, and not by way of limitation, the detailed description of the invention is divided into the following subsections:

(i) Thymus derived peptides

(ii) Uses of the Compositions of the Invention

(iii) Infectious Disease

(iv) Transplant/Graft Rejection

(v) Autoimmune Disease

(vi) Cancer

(vii) Alzheimer's Disease

(viii) Allergic Disease

(ix) Characterization and Demonstration of thymus derived peptide activities

(x) Dosage Regimens

(xi) Administrations, Formulations

(xii) Preparation of Peptides (Purification, Recombinant, Peptide Synthesis)

(xiii) Articles of Manufacture

### (i) Thymus Derived Peptides

The present invention includes in some aspects a composition suitable for administration to humans containing one or more specific peptides referred to herein as thymus derived peptides. The thymus derived peptides are present in subfractions of extracts obtained from thymus and have sometimes been described as “thymus nuclear protein (TNP)” or “thymus factors (TF)” when isolated from calf thymus (see for example US 20040018639). TNP or TF refers to those proteins that are produced in and found in the thymus. The peptides contributing to the therapeutic activity of TNP have now been identified and characterized and are useful for therapeutic purposes such as the treatment of infectious disease, cancer, autoimmune disease, Alzheimer's disease and transplant/graft rejection. These thymus derived peptides are described structurally in Table 1.

TNPs are typically purified from the thymus cells of freshly sacrificed, i.e., 4 hours or less after sacrifice, mammals such as monkeys, gorillas, chimpanzees, guinea pigs, cows, rabbits, dogs, mice and rats. Such methods can also be used to prepare a preparation of peptides of the invention. Alternatively, the thymus derived peptides can be synthesized using routine procedures known in the art in view of the peptide sequence information provided in Table 1. Such methods are preferred in some embodiments and such peptides are referred to herein as synthetic peptides. For instance, it is routine in to the art to prepare peptides using recombinant technology. Additionally the peptides may be purchased from commercial vendors that synthesize proteins or they may be synthesized directly using known techniques for peptide synthesis. Each of these methods is described in more detail below.

The compositions include one or more of the thymus derived peptides listed in Table 1. The compositions for therapeutic use can include, one or more, most or all of the peptides found in Table 1 as long as the composition is not a thymus nuclear protein extract or TNP extract. As used herein a “thymus nuclear protein extract” or “TNP extract” is a preparation of thymus peptides isolated and formulated according to the methods described in U.S. Ser. No. 11/973,920. A composition is not a thymus nuclear protein extract or TNP extract if it has additional components or less components or is all or partly synthetic. For instance a composition is not a thymus nuclear protein extract or TNP extract if the peptides included therein are prepared from natural sources but the composition does not include every peptide of a thymus nuclear protein extract as described in U.S. Ser. No. 11/973,920, for instance those listed in Table 1. Thus a single composition may include many of these peptides as long as all of the peptides found in Table 1 are not included if all of the peptides are derived from a natural thymus. However, the composition may include all of the peptides if one or more of the peptides in the mixture are synthetic. Additionally, it may include all of the peptides if one or more additional elements is added such as an extra synthetic peptide.

When the composition includes more than one thymus derived peptide, the ratio of the peptides in the composition can vary greatly. For instance if the composition includes two different peptides the ratio of the first peptide to the second peptide can range from 0.01 weight percent (wt %): 0.99 wt % to 0.99 wt %:0.1 wt % or any ratio there between.

In some instances the composition includes cystatin A and/or histones and in other instances the composition is free of cystatin A or histones. Histone encompasses all histone proteins including HI, H2A, H2B, H3, H4 and H5.

TABLE 1

| Amino Acid Sequence | SEQ ID NO. |
|---|---|
| KALVQNDTLLQVKG | 1 |
| KAMDIMNSFVNDIFERI | 2 |
| KAMGIMKSFVNDIFERI | 3 |
| KAMGNMNSFVNDIFERI | 4 |
| KAMSIMNSFVNDLFERL | 5 |
| KASGPPVSELITKA | 6 |
| KDAFLGSFLYEYSRR | 7 |
| KDDPHACYSTVFDKL | 8 |
| KEFFQSAIKLVDFQDAKA | 9 |
| KESYSVYVYKV | 10 |
| KGLVLIAFSQYLQQCPFDEHVKL | 11 |
| KHLVDEPQNLIKQ | 12 |
| KHPDSSVNFAEFSKK | 13 |
| KKQTALVELLKH | 14 |
| KKVPEVSTPTLVEVSRN | 15 |
| KLFTFHADICTLPDTEKQ | 16 |
| KLGEYGFQNALIVRY | 17 |
| KLKPDPNTLCDEFKA | 18 |
| KLVNELTEFAKT | 19 |
| KLVVSTQTALA | 20 |
| KQTALVELLKH | 21 |
| KSLHTLFGDELCKV | 22 |
| KTITLEVEPSDTIENVKA | 23 |
| KTVMENFVAFVDKC | 24 |
| KTVMENFVAFVDKCCAADDKEACFAVEGPKL | 25 |
| KTVTAMDVVYALKR | 26 |
| KVFLENVIRD | 27 |
| KVPEVSTPTLVEVSRN | 28 |
| KYLYEIARR | 29 |
| MGIMNSFVNDIFERI | 30 |
| RAGLQFPVGRV | 31 |
| RDNIQGITKPAIRR | 32 |
| REIAQDFKTDLRF | 33 |
| RFQSAAIGALQEASEAYLVGLFEDTNLCAIHAKR | 34 |
| RILGLIYEETRR | 35 |
| RISGLIYEETRG | 36 |
| RISGLIYKETRR | 37 |
| RKENHSVYVYKV | 38 |
| RLLLPGELAKH | 39 |
| RNDEELNKLLGKV | 40 |
| RNECFLSHKDDSPDLPKL | 41 |
| RRPCFSALTPDETYVPKA | 42 |
| RTLYGFGG | 43 |
| RTSKLQNEIDVSSREKS | 44 |
| RVTIAQGGVLPNIQAVLLPKK | 45 |
| LPDTEKQKL | 46 |
| YSTVFDKLK | 47 |
| ITLEVEPSD | 48 |
| LVQNDTLLQ | 49 |
| IKAMGIMKS | 50 |
| IKAMSIMNS | 51 |
| YVYKVRLLL | 52 |
| IKAMGNMNS | 53 |
| VRLLLPGEL | 54 |
| VVYALKRKV | 55 |
| YEIARRMGI | 56 |
| FRFQSAAIG | 57 |
| VVSTQTALA | 58 |
| IMNSFVNDI | 59 |
| ICTLPDTEK | 60 |
| MGIMKSFVN | 61 |
| MGIMNSFVN | 62 |
| LVELLKHKS | 63 |
| FERIKAMGI | 64 |
| FERIKAMSI | 65 |
| VLIAFSQYL | 66 |
| IMNSFVNDL | 67 |
| IMKSFVNDI | 68 |
| IQGITKPAI | 69 |
| VYVYKVRLL | 70 |
| YVYKVKGLV | 71 |
| LIYKETRRR | 72 |
| VKGLVLIAF | 73 |
| IRRREIAQD | 74 |
| VYVYKVKGL | 75 |
| VTAMDVVYA | 76 |
| YGFQNALIV | 77 |
| LVNELTEFA | 78 |
| VRYKLKPDP | 79 |
| LKTVTAMDV | 80 |
| FQNALIVRY | 81 |
| MSIMNSFVN | 82 |
| VKAKTVMEN | 83 |
| FKAKLVNEL | 84 |
| LRFRFQSAA | 85 |
| LVLIAFSQY | 86 |
| LKASGPPVS | 87 |
| VIRDKVPEV | 88 |
| VQNDTLLQV | 89 |
| MGNMNSFVN | 90 |
| YVPKARTLY | 91 |
| FQSAIKLVD | 92 |
| LYGFGGRTS | 93 |
| YKVKGLVLI | 94 |
| LVELLKHKK | 95 |
| LKHKKVPEV | 96 |
| LLKHKSLHT | 97 |
| YKVRLLLPG | 98 |
| VRNECFLSH | 99 |
| IVRYKLKPD | 100 |
| LIVRYKLKP | 101 |
| LLGKVRNEC | 102 |
| FERIKAMGN | 103 |
| VAFVDKCCA | 104 |
| LIYEETRRR | 105 |
| LIYEETRGR | 106 |
| VYALKRKVF | 107 |
| YLYEIARRM | 108 |
| LVVSTQTAL | 109 |
| VFLENVIRD | 110 |
| LVEVSRNKL | 111 |
| LIAFSQYLQ | 112 |
| IRDKVPEVS | 113 |
| LCKVKTITL | 114 |
| LIKQKHPDS | 115 |
| FERIRAGLQ | 116 |
| FQSAAIGAL | 117 |
| LVEVSRNKY | 118 |
| VKLKHLVDE | 119 |
| VYKVKGLVL | 120 |
| YALKRKVFL | 121 |
| VELLKHKKV | 122 |
| LQVKGKAMD | 123 |
| LKHKSLHTL | 124 |
| VELLKHKSL | 125 |
| VPKARTLYG | 126 |
| FKTDLRFRF | 127 |
| MDIMNSFVN | 128 |
| IKLVDFQDA | 129 |
| FVDKCKTVM | 130 |
| IHAKRRILG | 131 |
| FLYEYSRRK | 132 |
| VMENFVAFV | 133 |
| YLVGLFEDT | 134 |
| VYKVRLLLP | 135 |
| YLQQCPFDE | 136 |
| IRAGLQFPV | 137 |
| LLKHKKVPE | 138 |
| IKQKHPDSS | 139 |
| VLPNIQAVL | 140 |
| VEPSDTIEN | 141 |
| FGGRTSKLQ | 142 |
| VAFVDKCKT | 143 |
| FFQSAIKLV | 144 |
| FQDAKAKES | 145 |
| IQAVLLPKK | 146 |
| LLQVKGKAM | 147 |
| IAFSQYLQQ | 148 |
| FLGSFLYEY | 149 |
| FVNDIFERI | 150 |
| VDEPQNLIK | 151 |
| LSHKDDSPD | 152 |
| FLSHKDDSP | 153 |
| LPNIQAVLL | 154 |
| LKRKVFLEN | 155 |
| LLPGELAKH | 156 |
| FVAFVDKCC | 157 |
| IFERIKAMS | 158 |
| IENVKAKTV | 159 |
| VSRNKLFTF | 160 |
| LKPDPNTLC | 161 |
| MENFVAFVD | 162 |
| YSRRKDDPH | 163 |
| LFGDELCKV | 164 |
| FERLKASGP | 165 |
| VSTQTALAK | 166 |
| FAKTKLVVS | 167 |
| VTIAQGGVL | 168 |
| LNKLLGKVR | 169 |
| LYEIARRMG | 170 |
| MKSFVNDIF | 171 |
| LFTFHADIC | 172 |
| LAKQTALVE | 173 |
| FVAFVDKCK | 174 |
| FVNDLFERL | 175 |
| VKTITLEVE | 176 |
| IAQGGVLPN | 177 |
| LRRPCFSAL | 178 |
| LGSFLYEYS | 179 |
| LCAIHAKRR | 180 |
| LPKLRRPCF | 181 |
| VEVSRNKLF | 182 |
| FLENVIRDK | 183 |
| IYKETRRRK | 184 |
| VEVSRNKYL | 185 |
| FVDKCCAAD | 186 |
| LFEDTNLCA | 187 |
| VNFAEFSKK | 188 |
| VGRVRDNIQ | 189 |
| MNSFVNDIF | 190 |
| MNSFVNDLF | 191 |
| LVDEPQNLI | 192 |
| FSKKKKQTA | 193 |
| YGFGGRTSK | 194 |
| LITKAKDAF | 195 |
| MDVVYALKR | 196 |
| LLLPGELAK | 197 |
| LQFPVGRVR | 198 |
| LKEFFQSAI | 199 |
| YEYSRRKDD | 200 |
| LTPDETYVP | 201 |
| LGKVRNECF | 202 |
| LKHLVDEPQ | 203 |
| LQNEIDVSS | 204 |
| LVDFQDAKA | 205 |
| FAVEGPKLK | 206 |
| VSELITKAK | 207 |
| IFERIRAGL | 208 |
| LENVIRDKV | 209 |
| VGLFEDTNL | 210 |
| VSSREKSRV | 211 |
| IYEETRRRI | 212 |
| IFERIKAMG | 213 |
| FGDELCKVK | 214 |
| LFERLKASG | 215 |
| IARRMGIMN | 216 |
| LGLIYEETR | 217 |
| ILGLIYEET | 218 |
| YEETRRRIS | 219 |
| IDVSSREKS | 220 |
| LHTLFGDEL | 221 |
| LVGLFEDTN | 222 |
| VKGKAMDIM | 223 |
| FPVGRVRDN | 224 |
| VSRNKYLYE | 225 |
| IAQDFKTDL | 226 |
| FHADICTLP | 227 |
| VRDNIQGIT | 228 |
| YKLKPDPNT | 229 |
| VDFQDAKAK | 230 |
| FAEFSKKKK | 231 |
| LYEYSRRKD | 232 |
| FDEHVKLKH | 233 |
| LTEFAKTKL | 234 |
| LQQCPFDEH | 235 |
| LEVEPSDTI | 236 |
| IGALQEASE | 237 |
| VDKCKTVME | 238 |
| VFDKLKEFF | 239 |
| FTFHADICT | 240 |
| VPEVSTPTL | 241 |
| FSALTPDET | 242 |
| ITKPAIRRR | 243 |
| YKETRRRKE | 244 |
| IYEETRGRI | 245 |
| VEGPKLKTV | 246 |
| FEDTNLCAI | 247 |
| VNELTEFAK | 248 |
| YSVYVYKVK | 249 |
| LQEASEAYL | 250 |
| ISGLIYKET | 251 |
| YEETRGRIS | 252 |
| FDKLKEFFQ | 253 |
| VSTPTLVEV | 254 |
| VNDLFERLK | 255 |
| LPGELAKHR | 256 |
| VNDIFERIK | 257 |
| FSQYLQQCP | 258 |
| ITKAKDAFL | 259 |
| LGEYGFQNA | 260 |
| LCDEFKAKL | 261 |
| VDKCCAADD | 262 |
| VNDIFERIR | 263 |
| ISGLIYEET | 264 |
| LAKHRNDEE | 265 |

In some embodiments, the compositions of the invention that are used in prevention or treatment of cancer and/or infectious diseases or other disorders comprise an enriched, an isolated, or a purified thymus derived peptide. In accordance with the methods described herein, a thymus derived peptide employed in a composition of the invention can be in the range of 0.001 to 100 percent of the total mg protein, or at least 0.001%, at least 0.003%, at least 0.01%, at least 0.1%, at least 1%, at least 10%, at least 30%, at least 60%, or at least 90% of the total mg protein. In one embodiment, a thymus derived peptide employed in a composition of the invention is at least 4% of the total to protein. In another embodiment, a thymus derived peptide is purified to apparent homogeneity, as assayed, e.g., by sodium dodecyl sulfate polyacrylamide gel electrophoresis.

The invention provides thymus derived peptides which can be from the thymus of various animals or synthesized. Thus, it would be valuable if the structure of other thymus derived peptides or fragments thereof may be predicted based on the amino acid sequence. Structure prediction, analysis of crystallographic data, sequence alignment, as well as homology modeling, can be accomplished using computer software programs available in the art, such as BLAST, CHARMm release 21.2 for the Convex, and QUANTA v. 3.3, (Molecular Simulations, Inc., York, United Kingdom).

The invention further provides derivatives (including but not limited to fragments), and analogs of the thymus derived peptides set forth in Table 1. The to production and use of derivatives and analogs related to thymus derived peptide are within the scope of the present invention. In a specific embodiment, the derivative or analog is functionally active, i.e., capable of exhibiting one or more functional activities associated with a full-length, wild-type thymus derived peptide.

In particular, thymus derived peptide derivatives can be made by altering thymus derived peptide sequences by substitutions, insertions or deletions that provide for functionally equivalent molecules. The thymus derived peptide derivatives of the invention include, but are not limited to, those containing, as a primary amino acid sequence, all or part of the amino acid sequence of a thymus derived peptide including altered sequences in which functionally equivalent amino acid residues are substituted for residues within the sequence resulting in a silent change (i.e., conservative substitutions). For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid, thymus derived peptide derivatives of the invention also include, but are not limited to, those containing, as a primary amino acid sequence, all or part of the amino acid sequence of a thymus derived peptide including altered sequences in which amino acid residues are substituted for residues with similar chemical properties (i.e., conservative substitutions). In specific embodiments, 1, 2, 3, 4, or 5 amino acids are substituted.

Derivatives or analogs of thymus derived peptide include, but are not limited to, those peptides which are substantially homologous to thymus derived peptide or fragments thereof.

Included within the scope of the invention are thymus derived peptide fragments or other derivatives or analogs which are differentially modified during or after translation, e.g., by glycosylation, acetylation, phosphorylation, amidation, derivatization by known protecting/blocking groups, proteolytic cleavage, linkage to an antibody to molecule or other cellular ligand, etc. Any of numerous chemical modifications may be carried out by known techniques, including but not limited to, reagents useful for protection or modification of free NH2-groups, free COOH-groups, OH-groups, side groups of Trp-, Tyr-, Phe-, His-, Arg-, or Lys-; specific chemical cleavage by cyanogen bromide, hydroxylamine, BNPS-Skatole, acid, or alkali hydrolysis; enzymatic cleavage by trypsin, chymotrypsin, papain, V8 protease, NaBH₄; acetylation, formylation, oxidation, reduction; metabolic synthesis in the presence of tunicamycin; etc.

Furthermore, if desired, nonclassical amino acids or chemical amino acid analogs can be introduced as a substitution or addition into the thymus derived peptide sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

In a specific embodiment, the thymus derived peptide derivative is a chimeric, or fusion, protein comprising a thymus derived peptide or fragment thereof fused via a peptide bond at its amino- and/or carboxy-terminus to a non-thymus derived peptide amino acid sequence. In an embodiment, the non-thymus derived peptide amino acid sequence is fused at the amino-terminus of a thymus derived peptide or a fragment thereof. In one embodiment, such a chimeric protein is produced by recombinant expression of a nucleic acid encoding the protein (comprising a thymus derived peptide-coding sequence joined in-frame to a non-thymus derived peptide coding sequence). Such a chimeric product can be custom made by a variety of companies (e.g., Retrogen, Operon, etc.) or made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. In a specific embodiment, such chimeric construction can be used to enhance one or more desired properties of a thymus derived peptide, including but not limited to, thymus derived peptide stability, solubility, or resistance to proteases. In another embodiment, chimeric construction can be used to target thymus derived peptide to a specific site, e.g., a chimeric construction comprising a thymus derived peptide fused to an antibody to a specific type of cancer allows thymus to derived peptide to be delivered to the cancer site. In yet another embodiment, chimeric construction can be used to identify or purify a thymus derived peptide of the invention, such as a His-tag, a FLAG tag, a green fluorescence protein (GFP), β-galactosidase, a maltose binding protein (MalE), a cellulose binding protein (CenA) or a mannose protein, etc.

The thymus derived peptide sequence can be characterized by a hydrophilicity analysis (Hopp, T. and Woods, K., 1981, Proc. Natl. Acad. Sci. U.S.A. 78: 3824). A hydrophilicity profile can be used to identify the hydrophobic and hydrophilic regions of the thymus derived peptide.

Secondary structural analysis (Chou, P. and Fasman, G., 1974, Biochemistry 13: 222) can also be done, to identify regions of the thymus derived peptide that assume specific secondary structures.

Other methods of structural analysis can also be employed. These include, but are not limited to, X-ray crystallography (Engstom, A., 1974, Biochem. Exp. Biol. 11: 7-13) and computer modeling (Fletterick, R. and Zoller, M. (eds.), 1986, Computer Graphics and Molecular Modeling, in Current Communications in Molecular Biology, Cold Spring Harbor Laboratory, Cold Spring Harbor, N.Y.).

The functional activity of a thymus derived peptide or a fragment thereof can be assayed by various methods known in the art.

The peptides useful herein are isolated peptides. As used herein, the term “isolated” means that the referenced material is removed from its native environment, e.g., a cell. Thus, an isolated biological material can be free of some or all cellular components, i.e., components of the cells in which the native material is occurs naturally (e.g., cytoplasmic or membrane component). The isolated peptides may be substantially pure and essentially free of other substances with which they may be found in nature or in vivo systems to an extent practical and appropriate for their intended use. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from at least one of the substances with which it may be associated in living systems.

The term “purified” in reference to a protein or a nucleic acid, refers to the separation of the desired substance from contaminants to a degree sufficient to allow the practitioner to use the purified substance for the desired purpose. Preferably this means at least one order of magnitude of purification is achieved, more preferably two or three orders of magnitude, most preferably four or five orders of magnitude of purification of the starting material or of the natural material. In specific embodiments, a purified thymus derived peptide is at least 60%, at least 80%, or at least 90% of total protein or nucleic acid, as the case may be, by weight. In a specific embodiment, a purified thymus derived peptide is purified to homogeneity as assayed by, e.g., sodium dodecyl sulfate polyacrylamide gel electrophoresis, or agarose gel electrophoresis.

### (ii) Uses of the Compositions of the Invention

The composition of the current invention which contains one or more thymus derived peptides is of interest to the therapeutic treatment of numerous diseases. When the sub-fractions of TNP extracts containing such peptides are administered to a diseased individual, it improves health over time compared to untreated individuals. In particular, individuals having received the sub-fractions of TNP extracts containing such peptides display increases in the number of T_{H} cells compared to untreated individuals as well as show various improvements in disease condition.

The thymus derived peptides, thus, in some embodiments exhibit increases in T_{H} cells of at least 10%, 25%, 40%, 50%, 60%, 70%, 80%, 90%, 100% or more. In other embodiments the thymus derived peptides increase weight gain of a subject by 0.1-1 kg, 1-2 kg, 2-3 kg or more than 3 kg.

For patients suffering from a viral or retroviral infection, treatment with the composition of the current invention can effect a reduction in viral load of at least 10%, 25%, 40%, 50%, 60%, 70%, 75%, 80%, 90%, 100% or more.

Furthermore, the effects obtained by treatment with the composition of the current invention are maintained for at least 90 days, 150 days, 180 days, 240 days, 330 days, 667 days or more after conclusion of treatment.

Thus, in some aspects the invention relates to a method for treating a disorder associated with a decrease in the T_{H} cell number by administering to a subject a thymus derived peptide in an effective amount to treat the disease or to inhibit the decrease in T_{H} cell number. A disorder associated with decrease in the T_{H} cell number is one in which the levels of T_{H} cells are decreased below a normal level in the absence of the disease. An example of a disorder associated with decrease in the T_{H} cell number is HIV infection. It is believed that, according to an aspect of the invention, the thymus derived peptides of the invention can interact with cell surface proteins and protect T_{H} cells from harmful interactions with other cells. Other disorders associated with decrease in the T_{H} cell number include autoimmune disease, cancer, Alzheimer's disease and rejection of transplanted cells, tissues or grafts. The loss of host T cells is critical in advancing the HIV infection.

A subject shall mean a human or vertebrate mammal including but not limited to a dog, cat, horse, goat and primate, e.g., monkey. Thus, the invention can also be used to treat diseases or conditions in non human subjects. Preferably the subject is a human.

As used herein, the term treat, treated, or treating when used with respect to a disorder refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy.

When used in combination with the therapies of the invention the dosages of known therapies may be reduced in some instances, to avoid side effects.

The thymus derived peptide can be administered in combination with other therapeutic agents and such administration may be simultaneous or sequential. When the other therapeutic agents are administered simultaneously they can be administered in the same or separate formulations, but are administered at the same time. The administration of the other therapeutic agent and the thymus derived peptide can also be temporally separated, meaning that the therapeutic agents are administered at a different time, either before or after, the administration of the thymus derived peptide. The separation in time between the administration of these compounds may be a matter of minutes or it may be longer.

(iii) Infectious Disease

Infectious diseases that can be treated or prevented by the methods of the present to invention are caused by infectious agents including, but not limited to, viruses, bacteria, fungi, protozoa and parasites.

The present invention provides methods of preventing or treating an infectious disease, by administering to a subject in need thereof a composition comprising thymus derived peptide alone or in combination with one or more prophylactic or therapeutic agents other than the thymus derived peptide. Any agent or therapy which is known to be useful, or which has been used or is currently being used for the prevention or treatment of infectious disease can be used in combination with the composition of the invention in accordance with the methods described herein.

Viral diseases that can be treated or prevented by the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papolomavirus, cytomegalovirus, echinovirus, arbovirus, huntavirus, coxsackie virus, mumps virus, measles virus, rubella virus, and polio virus. In accordance with the some preferred embodiments of the invention, the disease that is treated or prevented by the methods of the present invention is caused by a human immunodeficiency virus (human immunodeficiency virus type I (HIV-I), or human immunodeficiency virus type II (HIV-II); e.g., the related disease is AIDS). In other embodiments the disease that is treated or prevented by the methods of the present invention is caused by a Herpes virus, Hepatitis virus, Borrelia virus, Cytomegalovirus, or Epstein Barr virus.

AIDS or HIV Infection

According to an embodiment of the invention, the methods described herein are useful in treating AIDS or HIV infections. HIV stands for human immunodeficiency virus, the virus that causes AIDS. HIV is different from many other viruses because it attacks the immune system, and specifically white blood cell (T cells or CD4 cells) that are important for the immune system to fight disease. In a specific embodiment, treatment is by introducing one or more thymus derived peptides into a subject infected with HIV. In particular, HIV intracellular entry into T cells can be blocked by treatment with the peptides of the invention.

Both B cell and T cell populations undergo dramatic changes following HIV-infection. During the early stages of HIV infection, peripheral B-cells undergo aberrant polyclonal activation in an antigen-independent manner [Lang, K. S., et al., Toll-like receptor engagement converts T-cell autoreactivity into overt autoimmune disease. Nat Med, 2005. 11(2): p. 138-45.], perhaps as a consequence of their activation by HIV gp120 (He, B., et al., HIV-1 envelope triggers polyclonal Ig class switch recombination through a CD40-independent mechanism involving BAFF and C-type lectin receptors. J Immunol, 2006. 176(7): p. 3931-41.). At early stages, the B cells appear to be resistant to T cell-mediated cytotoxicity [Liu, J. and M. Roederer, Differential susceptibility of leukocyte subsets to cytotoxic T cell killing: implications for HIV immunopathogenesis. Cytometry A, 2007. 71(2): p. 94-104]. However, later in infection, perhaps as a direct consequence of their antigen-independent activation [Cambier, J. C., et al., Differential transmembrane signaling in B lymphocyte activation. Ann N Y Acad Sci, 1987. 494: p. 52-64. Newell, M. K., et al., Ligation of major histocompatibility complex class II molecules mediates apoptotic cell death in resting B lymphocytes. Proc Natl Acad Sci U S A, 1993. 90(22): p. 10459-63], B-cells become primed for apoptosis [Ho, J., et al., Two overrepresented B cell populations in HIV-infected individuals undergo apoptosis by different mechanisms. Proc Natl Acad Sci USA, 2006. 103(51): p. 19436-41]. The defining characteristic of HIV infection is the depletion of CD4+ T-cells. A number of mechanisms may contribute to killing, including direct killing of the infected CD4+ T-cells by the virus or “conventional” killing of HIV-infected cells by cytotoxic CD8+ lymphocytes. The effectiveness of cytotoxic T cell killing is dramatically impaired by down-regulation of class I MHC expression on the surface of the infected cell due to the action of the viral Tat and Nef proteins [Joseph, A. M., M. Kumar, and D. Mitra, Nef: “necessary and enforcing factor” in HIV infection. Curr HIV Res, 2005. 3(1): p. 87-94.]. However, the same reduction in MHC class I expression that impairs cytotoxic T-cell mediated killing, in conjunction with increased expression of death inducing receptors, could mark infected cells, such as CD4⁺ macrophages and CD4⁺ T cells, instead as targets for NK or γδ T cell killing.

Recent work suggests that HIV-1 infection leads to a broad level of chronic activation of the immune system including changes in cytokines, redistribution of lymphocyte subpopulations, immune cell dysfunctions, and cell death [Biancotto, A., et al., Abnormal activation and cytokine spectra in lymph nodes of people chronically infected with HIV-1. Blood, 2007. 109(10): p. 4272-9.]. Our early work demonstrated that CD4 engagement prior to T cell receptor recognition of antigen and MHC class by CD4⁺ T cells primes CD4⁺ T cells for apoptotic cell death [Newell, M. K., et al., Death of mature T cells by separate ligation of CD4 and the T-cell receptor for antigen. Nature, 1990. 347(6290): p. 286-9]. As the CD4⁺ T cell levels decline, the ability to fight off minor infections declines, viremia increases, and symptoms of illness appear.

B cell activation is typically an exquisitely well-regulated process that requires interaction of the resting B cell with specific antigen. However, during the course of HIV infection, (and certain autoimmune diseases) peripheral B cells become polyclonally activated by an antigen-independent mechanism. Paradoxically, and in contrast to the polyclonal B cell activation and consequent hypergammaglobulinemia that is characteristic of early HIV infection, patients are impaired in their B cell response to immunological challenges, such as vaccination [Mason, R. D., R. De Rose, and S. J. Kent, CD4+ T-cell subsets: what really counts in preventing HIV disease? Expert Rev Vaccines, 2008. 7(2): p. 155-8]. At these early stages, the B cells appear to be resistant to T cell mediated cytotoxicity. At later stages in the course of infection, B cells from HIV infected patients become primed for apoptosis. The pathological role of polyclonal activated B cells and late stage B cell death in HIV is not known.

There have been conflicting reports on the role of Tregs in HIV infection. Some argue that Tregs prevent an adequate CD4 T cell response to infections and that diminished Tregs may contribute directly, or indirectly to the loss of CD4 T cells. Others have recognized a positive correlation between decreases in Tregs and viremia and advancing disease. These seemingly opposing functions of Tregs can likely be reconciled by the fact that HIV infection renders Tregs dysfunctional at two stages of disease: early Treg dysfunction prevents B cell death of polyclonally activated B cells and, in late stage disease, HIV-induced death of Treg correlates with late stage conventional CD4 T cell activation and activation induced cell death resulting in loss of activated, conventional CD4T cells. Therefore an important therapeutic intervention of the invention involves reversal of Treg dysfunction in both early and late stages of disease. These methods may be accomplished using the thymus derived peptides of the invention. Although Applicant is not bound by a proposed mechanism of action, it is believed that the thymus derived peptides may be peptide targets for Treg activation. Therefore, polyclonally activated B cells, having self antigens in the groove of MHC class I or II, may serve as antigen presenting cells for the targeted peptides (thymus to derived peptides) such that the targeted peptides replace CLIP. This results in the activation of Tregs.

Susceptibility or resistance to many diseases appears to be determined by the genes encoding Major Histocompatibilty Complex (MHC) molecules. Often referred to as immune response genes (or IR genes), these molecules are the key players in restricting T cell activation. T cells, both CD8 and CD4 positive T cells, recognize antigens only when the antigen is presented to the T cell in association with MHC class I (expressed on all nucleated cells) or MHC class II molecules (expressed on cells that present antigens to CD4+ T cells), respectively. MHC molecules are highly polymorphic, meaning there are many possible alleles at a given MHC locus. The polymorphism of MHC accounts for the great variations in immune responses between individual members of the same species. The ability of an antigen to bind to the MHC molecules is therefore genetically dependent on the MHC alleles of the individual person.

Viral Genetics Inc. has conducted six human clinical trials outside of the United States testing the safety and efficacy of a TNP extract (TNP-1, referred to as VGV-1 in the trials) in patients infected with HIV. In all 6 studies, subjects received 8 mg VGV-1 as an intramuscular injection of 2.0 mL of a 4.0 mg/mL suspension of TNP, twice a week for 8 weeks for a total of 16 doses. The studies are described in detail in the Examples section. The data suggested that TNP-1 treatment in HIV-1 infected patients was safe and well tolerated in human trials. There was a decrease in CD4 cells observed in the trials which trended consistently with the natural progression of disease. However, changes in HIV-1 RNA observed were less than expected during a natural course of HIV-1 infection.

The South African study demonstrated efficacy of TNP in various subsets of HIV/AIDS patients while providing additional verification of the compound being well-tolerated. In brief, TNP appeared to have a meaningful effect on levels of HIV virus in subsets of patients with more heavily damaged immune systems. The discoveries of the invention, specifically relating to thymus derived peptides are consistent with and provide an explanation for some of the observations arising in the trials. For instance, the fact that TNP which has long been believed to be an immune-based drug, showed superior results in patients with a more damaged immune system was difficult to reconcile. However, the results of the invention specifically related to the ability of thymus derived peptides to reverse Treg dysfunction in HIV disease, as discussed above.

Additionally, the transient, short-term anti-HIV effect of TNP in the clinical trials was difficult to explain. The results of the instant invention demonstrate that these results appears to be a simple dosing problem. The formulation used in the clinical trials was not the ideal dosage and the number of times it is administered was also likely not optimal. By extending the period of time TNP is dosed and increasing the dosage, it appears likely it can achieve a longer-lasting effect.

Another phenomena observed in the clinical trial related to the fact that TNP appeared to work in 25-40% of patients. The discoveries of the invention provide an explanation for this. It has been discovered that TNP includes several protein compounds that should be able to treat HIV in certain subgroups of human patients but not all of them. This is based on the specific MHC of the patient. The invention also relates to the discovery of subgroups of peptides that are MHC matched that will provide more effective treatment for a much larger group of patients. The differential binding affinity of the TNP peptides to widely variant MHC molecules between individuals may account for the variation in the ability of TNP peptides to modulate disease between various HIV-infected people. MHC polymorphisms may also account for the wide range that describes time between first infection with HIV and the time to onset of full-blown AIDS.

Because TNP is derived from the thymus, the epitopes in the TNP mixtures could be involved in Treg selection. The B cell would not be recognized by the Tregs until TNP peptides (thymus derived peptides), or other appropriate self peptides, competitively replace the endogenous peptide in the groove of B cell MHC class II. The TNP peptides are likely enriched for the pool that selects Tregs in the thymus and these peptides are processed and presented in B cells differentially depending on disease state. Therefore, the partial success in reducing the HIV viral load that was observed in patients treated with the VGV-1 targeted peptide treatment is explained by the following series of observations: 1) gp120 from HIV polyclonally activates B cells that present conserved self antigens via MHC class II (or potentially MHC class I) and the activated B cells stimulate gamma delta T cells, 2) the VGV-1 targeted peptides bind with stronger affinity to the MHC molecules of the polyclonally activated B cell, 3) the consequence is activation and expansion of Tregs whose activation and expansion corresponds with decreased viral load, diminished γδ T cell activation, and improvement as a result of to inhibition of activation-induced cell death of non-Treg (referred to as conventional) CD4+ T cells.

The discoveries of the invention suggest that the success of TNP extract treatment in HIV patients involves binding of targeted peptides from the TNP mixture to cell surface Major Histocompatibility Complex (MHC) molecules on the activated B cell surface. MHC molecules are genetically unique to individuals and are co-dominantly inherited from each parent. MHC molecules serve to display newly encountered antigens to antigen-specific T cells. According to our model, if the MHC molecules bind a targeted peptide that has been computationally predicted to bind the individual's MHC molecules with greater affinity than the peptide occupying the groove of the MHC molecules on the activated B cell surface, the consequence will be activation of Treg cells that can dampen an inflammatory response. Tregs usually have higher affinity for self and are selected in the thymus. Because TNP is derived from the thymus, it is reasonable to suggest that these epitopes could be involved in Treg selection. Aberrantly activated B cells have switched to expression of non-thymically presented peptides. The TNP peptides may be represented in the pool that selects Tregs in the thymus. Loading of the thymic derived peptides onto activated B cells then provides a unique B cell/antigen presenting cell to activate the Treg.

In accordance with another embodiment, the methods of this invention can be applied in conjunction with, or supplementary to, the customary treatments of AIDS or HIV infection. Historically, the recognized treatment for HIV infection is nucleoside analogs, inhibitors of HIV reverse transcriptase (RT). Intervention with these antiretroviral agents has led to a decline in the number of reported AIDS cases and has been shown to decrease morbidity and mortality associated with advanced AIDS. Prolonged treatment with these reverse transcriptase inhibitors eventually leads to the emergence of viral strains resistant to their antiviral effects. Recently, inhibitors of HIV protease have emerged as a new class of HIV chemotherapy. HIV protease is an essential enzyme for viral infectivity and replication. Protease inhibitors have exhibited greater potency against HIV in vitro than nucleoside analogs targeting HIV-1 RT. Inhibition of HIV protease disrupts the creation of mature, infectious virus particles from chronically infected cells. This enzyme has become a viable target for therapeutic intervention and a candidate for combination therapy.

Knowledge of the structure of the HIV protease also has led to the development of novel inhibitors, such as saquinovir, ritonavir, indinivir and nelfinavir. NNRTIs (non-nucleoside reverse transcriptase inhibitors) have recently gained an increasingly important role in the therapy of HIV infection. Several NNRTIs have proceeded onto clinical development (i.e., tivirapine, loviride, MKC-422, HBY-097, DMP 266). Nevirapine and delaviridine have already been authorized for clinical use. Every step in the life cycle of HIV replication is a potential target for drug development.

Many of the antiretroviral drugs currently used in chemotherapy either are derived directly from natural products, or are synthetics based on a natural product model. The rationale behind the inclusion of deoxynucleoside as a natural based antiviral drugs originated in a series of publications dating back as early as 1950, wherein the discovery and isolation of thymine pentofuranoside from the air-dried sponges (Cryptotethia crypta) of the Bahamas was reported. A significant number of nucleosides were made with regular bases but modified sugars, or both acyclic and cyclic derivatives, including AZT and acyclovir. The natural spongy-derived product led to the first generation, and subsequent second—third generations of nucleosides (AZT, DDI, DDC, D4T, 3TC) antivirals specific inhibitors of HIV-1 RT.

A number of non-nucleoside agents (NNRTIs) have been discovered from natural products that inhibit RT allosterically. NNRTIs have considerable structural diversity but share certain common characteristics in their inhibitory profiles. Among NNRTIs isolated from natural products include: calanoid A from calophylum langirum; Triterpines from Maporonea African a. There are publications on natural HIV integrase inhibitors from the marine ascidian alkaloids, the lamellarin.

Lyme's Disease is a tick-borne disease caused by bacteria belonging to the genus Borrelia. Borrelia burgdorferi is a predominant cause of Lyme disease in the US, whereas Borrelia afzelii and Borrelia garinii are implicated in some European countries. Early manifestations of infection may include fever, headache, fatigue, and a characteristic skin rash called erythema migrans. Long-term the disease involves malfunctions of the joints, heart, and nervous system. Currently the disease is treated with antibiotics. The antibiotics generally used for the treatment of the disease are doxycycline (in adults), amoxicillin (in children), and ceftriaxone. Late, delayed, or inadequate treatment can lead to late manifestations of Lyme disease which can be disabling and difficult to treat.

A vaccine, called Lymerix, against a North American strain of the spirochetal to bacteria was approved by the FDA and leter removed from the market. It was based on the outer surface protein A (OspA) of B. burgdorferi. It was discovered that patients with the genetic allele HLA-DR4 were susceptible to T-cell cross-reactivity between epitopes of OspA and lymphocyte function-associated antigen in these patients causing an autoimmune reaction.

It is believed according to the invention that Borrelia Bergdorf also produces a Toll ligand for TLR2. Replacement of the CLIP on the surface of the B cell by treatment with a thymus derived peptide with high affinity for the MHC fingerprint of a particular individual, would result in activation of the important Tregs that can in turn cause reduction in antigen-non-specific B cells. Thus treatment with thymus derived peptides could reactivate specific Tregs and dampen the pathological inflammation that is required for the chronic inflammatory condition characteristic of Lyme Disease. With the appropriate MHC analysis of the subject, a specific thymus derived peptide can be synthesized to treat that subject. Thus individuals with all different types of MHC fingerprints could effectively be treated for Lymes disease.

Chronic Lyme disease is sometimes treated with a combination of a macrolide antibiotic such as clarithromycin (biaxin) with hydrochloroquine (plaquenil). It is thought that the hydroxychloroquine raises the pH of intracellular acidic vacuoles in which B. burgdorferi may reside; raising the pH is thought to activate the macrolide antibiotic, allowing it to inhibit protein synthesis by the spirochete.

At least four of the human herpes viruses, including herpes simplex virus type 1 (HSV-1), herpes simplex virus type 2 (HSV-2), cytomegalovirus (CMV), Epstein-Barr virus (EBV), and varicella zoster virus (VZV) are known to infect and cause lesions in tissues of certain infected individuals. Infection with the herpes virus is categorized into one of several distinct disorders based on the site of infection. For instance, together, these four viruses are the leading cause of infectious blindness in the developed world. Oral herpes, the visible symptoms of which are referred to as cold sores, infects the face and mouth. Infection of the genitals, commonly known as, genital herpes is another common form of herpes. Other disorders such as herpetic whitlow, herpes gladiatorum, ocular herpes (keratitis), cerebral herpes infection encephalitis, Mollaret's meningitis, and neonatal herpes are all caused by herpes simplex viruses. Herpes simplex is most easily transmitted by direct contact with a lesion or the body fluid of an infected individual. Transmission may also occur through skin-to-skin contact during periods of asymptomatic shedding.

HSV-1 primarily infects the oral cavity, while HSV-2 primarily infects genital sites. However, any area of the body, including the eye, skin and brain, can be infected with either type of HSV. Generally, HSV is transmitted to a non-infected individual by direct contact with the infected site of the infected individual.

VZV, which is transmitted by the respiratory route, is the cause of chickenpox, a disease which is characterized by a maculopapular rash on the skin of the infected individual. As the clinical infection resolves, the virus enters a state of latency in the ganglia, only to reoccur in some individuals as herpes zoster or “shingles”. The reoccurring skin lesions remain closely associated with the dermatome, causing intense pain and itching in the afflicted individual.

CMV is more ubiquitous and may be transmitted in bodily fluids. The exact site of latency of CMV has not been precisely identified, but is thought to be leukocytes of the infected host. Although CMV does not cause vesicular lesions, it does cause a rash. Human CMVs (HCMV) are a group of related herpes viruses. After a primary infection, the viruses remain in the body in a latent state. Physical or psychic stress can cause reactivation of latent HCMV. The cell-mediated immune response plays an important role in the control and defense against the HCMV infection. When HCMV-specific CD8⁺ T cells were transferred from a donor to a patient suffering from HCMV, an immune response against the HCMV infection could be observed (P. D. Greenberg et al., 1991, Development of a treatment regimen for human cytomegalovirus (CMV) infection in bone marrow transplantation recipients by adoptive transfer of donor-derived CMV-specific T cell clones expanded in vitro. Ann N.Y. Acad. Sci., Vol.: 636, pp 184 195). In adults having a functional immune system, the infection has an uneventful course, at most showing non-specific symptoms, such as exhaustion and slightly increased body temperature. Such infections in young children are often expressed as severe respiratory infection, and in older children and adults, they are expressed as anicteric hepatitis and mononucleosis. Infection with HCMV during pregnancy can lead to congenital malformation resulting in mental retardation and deafness. In immunodeficient adults, pulmonary diseases and retinitis are associated with HCMV infections.

Epstein-Barr virus frequently referred to as EBV, is a member of the herpesvirus family and one of the most common human viruses. The virus occurs worldwide, and most people become infected with EBV sometime during their lives. Many children become infected with EBV, and these infections usually cause no symptoms or are indistinguishable from the other mild, brief illnesses of childhood. When infection with EBV occurs during adolescence or young adulthood, it can cause infectious mononucleosis. EBV also establishes a lifelong dormant infection in some cells of the body's immune system. A late event in a very few carriers of this virus is the emergence of Burkitt's lymphoma and nasopharyngeal carcinoma, two rare cancers that are not normally found in the United States. EBV appears to play an important role in these malignancies, but is probably not the sole cause of disease.

No treatment that can eradicate herpes virus from the body currently exists. Antiviral medications can reduce the frequency, duration, and severity of outbreaks. Antiviral drugs also reduce asymptomatic shedding. Antivirals used against herpes viruses work by interfering with viral replication, effectively slowing the replication rate of the virus and providing a greater opportunity for the immune response to intervene. Antiviral medicaments for controlling herpes simplex outbreaks, include aciclovir (Zovirax), valaciclovir (Valtrex), famciclovir (Famvir), and penciclovir. Topical lotions, gels and creams for application to the skin include Docosanol (Avanir Pharmaceuticals), Tromantadine, and Zilactin.

Various substances are employed for treatment against HCMV. For example, Foscarnet is an antiviral substance which exhibits selective activity, as established in cell cultures, against human herpes viruses, such as herpes simplex, varicella zoster, Epstein-Barr and cytomegaloviruses, as well as hepatitis viruses. The antiviral activity is based on the inhibition of viral enzymes, such as DNA polymerases and reverse transcriptases.

Hepatitis refers to inflammation of the liver and hepatitis infections affect the liver. The most common types are hepatitis A, hepatitis B, and hepatitis C. Hepatitis A is caused by the hepatitis A virus (HAV) and produces a self-limited disease that does not result in chronic infection or chronic liver disease. HAV infection is primarily transmitted by the fecal-oral route, by either person-to-person contact or through consumption of contaminated food or water. Hepatitis B is a caused by hepatitis B virus (HBV) and can cause acute illness, leading to chronic or lifelong infection, cirrhosis (scarring) of the liver, liver cancer, liver failure, and death. HBV is transmitted through percutaneous (puncture through the skin) or mucosal contact with infectious blood or body fluids. Hepatitis C is caused by the hepatitis C virus (HCV) that sometimes results in an acute illness, but most often becomes a silent, chronic infection that can lead to cirrhosis, liver failure, liver cancer, and death. Chronic HCV infection develops in a majority of HCV-infected persons. HCV is spread by contact with the blood of an infected person.

Presently, the most effective HCV therapy employs a combination of alpha-interferon and ribavirin. Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load.

Examples of antiviral agents that can be used in combination with thymus derived peptide to treat viral infections include, but not limited to, amantadine, ribavirin, rimantadine, acyclovir, famciclovir, foscarnet, ganciclovir, trifluridine, vidarabine, didanosine (ddI), stavudine (d4T), zalcitabine (ddC), zidovudine (AZT), lamivudine, abacavir, delavirdine, nevirapine, efavirenz, saquinavir, ritonavir, indinavir, nelfinavir, amprenavir, lopinavir and interferon.

Parasitic diseases that can be treated or prevented by the methods of the present invention are caused by parasites including, but not limited to, leishmania, and malaria. Hisaeda H. et al Escape of malaria parasites from host immunity requires CD4⁺CD25⁺ regulatory T cells Nature Medicine 10, 29-30 (2004) describes a study designed to understand why infection with malaria parasites frequently induced total immune suppression. Such immune suppression presents a challenge to the host in maintaining long-lasting immunity. Hisaeda et al demonstrated that depletion of T_{reg}s protected mice from death when infected with a lethal strain of Plasmodium yoelii, and that this protection was associated with an increased T-cell responsiveness against parasite-derived antigens. The authors concluded that “activation of T_{reg} cells contributes to immune suppression during malaria infection, and helps malaria parasites to escape from host immune responses.” Suffia I. J., et al Infected site-restricted Foxp3⁺ natural regulatory T cells are specific for microbial antigens, JEM, Volume 203, Number 3, 777-788 (2006) describe the finding that natural Treg cells are able to respond specifically to Leishmania. The majority of natural Treg cells at the infected site were Leishmania specific. The findings suggest that Leishmania induces Tregs to help dampen the immune response of the subject upon infection. Thus the methods of the invention are useful for treating parasitic infection by activating Tregs and preventing the immune suppression caused by such parasites.

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, timidazole, trimethroprim-sulfamethoxazole, and tryparsamide.

Bacterial diseases that can be treated or prevented by the methods of the present invention are caused by bacteria including, but not limited to, mycobacteria, rickettsia, mycoplasma, neisseria, Borrelia and legionella.

Although Applicant is not bound by a specific mechanism of action it is believed that the CLIP inhibitors of the invention displace CLIP from MHC class I and cause down regulation of Treg activity and/or activation of effector T cells such as γδ T cells. Downregulation of regulatory function of Treg activity prevents suppression of the immune response and enables the subject to mount an effective or enhanced immune response against the bacteria. At the same time the Treg cell may shift to an effector function, producing an antigen specific immune response. Thus, replacement of CLIP with a peptide of the invention results in the promotion of an antigen specific CD8+ response against the bacteria, particularly when the peptide is administered in conjunction with a tumor specific antigen. Activation of effector T cells also enhances the immune response against the bacteria, leading to a more effective treatment.

One component of the invention involves promoting an enhanced immune response against the bacteria by administering the compounds of the invention. The compounds may be administered in conjunction with an antigen to further promote a bacterial specific immune response. A “bacterial antigen” as used herein is a compound, such as a peptide or carbohydrate, associated with a bacteria surface and which is to capable of provoking an immune response when expressed on the surface of an antigen presenting cell in the context of an MHC molecule. Preferably, the antigen is expressed at the cell surface of the bacteria.

The compounds of the invention may be used in combination with anti-bacterial agents. Examples of such agents to treat bacterial infections include, but are not limited to, folate antagonists (e.g., mafenide, silver sulfadiazine, succinylsulfathiazole, sulfacetamide, sulfadiazine, sulfamethoxazole, sulfasalazine, sulfisoxazole, pyrimethoamine, trimethoprim, co-trimoxazole), inhibitors of cell wall synthesis (e.g., penicillins, cephalosporins, carbapenems, monobactams, vacomycin, bacitracin, clavulanic acid, sulbactam, tazobactam), protein synthesis inhibitors (e.g., tetracyclines, aminoglycosides, macrolides, chloramphenicol, clindamycin), fluoroquinolones (e.g., ciproloxacin, enoxacin, lomefloxacin, norfloxacin, ofloxacin), nalidixic acid, methenamine, nitrofurantoin, aminosalicylic acid, cycloserine, ethambutol, ethionamide, isoniazid, pyrazinamide, rifampin, clofazimine, and dapsone.

### (iv) Transplant/Graft Rejection

According to an embodiment of the invention, the methods described herein are useful in inhibiting cell graft or tissue graft rejection. Thus, the methods are useful for such grafted tissue as heart, lung, kidney, skin, cornea, liver, neuronal tissue or cell, or with stem cells, including hematopoietic or embryonic stem cells, for example.

The success of surgical transplantation of organs and tissue is largely dependent on the ability of the clinician to modulate the immune response of the transplant recipient. Specifically the immunological response directed against the transplanted foreign tissue must be controlled if the tissue is to survive and function. Currently, skin, kidney, liver, pancreas, lung and heart are the major organs or tissues with which allogeneic transplantations are performed. It has long been known that the normally functioning immune system of the transplant recipient recognizes the transplanted organ as “non-self” tissue and thereafter mounts an immune response to the presence of the transplanted organ. Left unchecked, the immune response will generate a plurality of cells and proteins that will ultimately result in the loss of biological functioning or the death of the transplanted organ.

This tissue/organ rejection can be categorized into three types: hyperacute, acute and chronic. Hyperacute rejection is essentially caused by circulating antibodies in the to blood that are directed against the tissue of the transplanted organ (transplant). Hyperacute rejection can occur in a very short time and leads to necrosis of the transplant. Acute graft rejection reaction is also immunologically mediated and somewhat delayed compared to hyperacute rejection. The chronic form of graft rejection that can occur years after the transplant is the result of a disease state commonly referred to as Graft Arterial Disease (GAD). GAD is largely a vascular disease characterized by neointimal proliferation of smooth muscle cells and mononuclear infiltrates in large and small vessels. This neointimal growth can lead to vessel fibrosis and occlusion, lessening blood flow to the graft tissue and resulting in organ failure. Current immunosuppressant therapies do not adequately prevent chronic rejection. Most of the gains in survival in the last decade are due to improvements in immunosuppressive drugs that prevent acute rejection. However, chronic rejection losses remain the same and drugs that can prevent it are a critical unmet medical need.

A clinical trial testing the use of Tregs obtained from umbilical cord blood to decrease the risk of immune reactions common in patients undergoing blood and marrow transplantation was recently initiated. It is expected that therapy will improve overall survival rates for blood cancer patients as well as offer a potential new mode for treating autoimmune diseases.

In a transplant situation, donor T-regs may suppress the recipient's immune system so that the healthy donor's blood-forming stem cells and immune cells can grow, helping ward off life-threatening graft-versus-host-disease (GVHD). GVHD occurs when the immune cells within the donated cells attack the body of the transplant recipient. In a recent study (Xia et al. Ex vivo-expanded natural CD4+CD25+ regulatory T cells synergize with host T-cell depletion to promote long-term survival of allografts. Am J Transplant. 2008 February; 8(2):298-306) the question of therapeutic utilization of T regulatory cells was asked in an animal model of heart transplantation. It was discovered that Tregs were capable of extending allograft survival in a donor specific manner.

The methods of the invention involve the specific activation of Tregs by replacement of the cell surface CLIP with a thymus derived peptide of the invention. This activation should result in a dampening of the immune system to suppress rejection of the graft.

The methods of treating transplant/graft rejection can be applied in conjunction with, or supplementary to, the customary treatments of transplant/graft rejection. Tissue to graft and organ transplant recipients are customarily treated with one or more cytotoxic agents in an effort to suppress the transplant recipient's immune response against the transplanted organ or tissue. Current immunosuppressant drugs include: cyclosporin, tacrolimus (FK506), sirolimus (rapamycin), methotrexate, mycophenolic acid (mycophenolate mofetil), everolimus, azathiprine, steroids and NOX-100. All of these drugs have side effects (detailed below) that complicate their long-term use. For example, cyclosporin (cyclosporin A), a cyclic polypeptide consisting of 11 amino acid residues and produced by the fungus species Tolypocladium inflatum Gams, is currently the drug of choice for administration to the recipients of allogeneic kidney, liver, pancreas and heart (i.e., wherein donor and recipient are of the same species of mammals) transplants. However, administration of cyclosporin is not without drawbacks as the drug can cause kidney and liver toxicity as well as hypertension. Moreover, use of cyclosporin can lead to malignancies (such as lymphoma) as well as opportunistic infection due to the “global” nature of the immunosuppression it induces in patients receiving long term treatment with the drug, i.e., the hosts normal protective immune response to pathogenic microorganisms is downregulated thereby increasing the risk of infections caused by these agents. FK506 (tacrolimus) has also been employed as an immunosuppressive agent as a stand-alone treatment or in combination. Although its immunosuppressive activity is 10-100 times greater than cyclosporin, it still has toxicity issues. Known side effects include kidney damage, seizures, tremors, high blood pressure, diabetes, high blood potassium, headache, insomnia, confusion, seizures, neuropathy, and gout. It has also been associated with miscarriages. Methotrexate is commonly added to the treatment of the cytotoxic agent. Methotrexate is given in small doses several times after the transplant. Although the combination of cyclosporin and methotrexate has been found to be effective in decreasing the severity of transplant rejection, there are side effects, such as mouth sores and liver damage. Severe transplant rejection can be treated with steroids. However, the side effects of steroids can be extreme, such as weight gain, fluid retention, elevated blood sugar, mood swings, and/or confused thinking

Rapamycin, a lipophilic macrolide used as an anti-rejection medication can be taken in conjunction with other anti-rejection medicines (i.e., cyclosporin) to reduce the amount of toxicity of the primary cytotoxic agent, but it too has specific side effects, to such as causing high cholesterol, high triglycerides, high blood pressure, rash and acne. Moreover, it has been associated with anemia, joint pain, diarrhea, low potassium and a decrease in blood platelets.

### (v) Autoimmune Disease

According to an embodiment of the invention, the methods described herein are useful in inhibiting the development of an autoimmune disease in a subject by administering a thymus derived peptide to the subject. Thus, the methods are useful for such autoimmune diseases as multiple sclerosis, systemic lupus erythematosus, type 1 diabetes, viral endocarditis, viral encephalitis, inflammatory bowel disease, rheumatoid arthritis, Graves' disease, autoimmune thyroiditis, autoimmune myositis, and discoid lupus erythematosus.

In autoimmune disease non-specifically activated B cells that do not undergo apoptosis are present. Although not being bound by a specific mechanism, it is believed that the thymus derived peptides of the invention result in activation of Tregs and reduction in these non-specific activated B cells. While, at first glance, it might seem immunologically dangerous to lose a majority of B cells for instance during an infection, it is noted that B cells continually mature in the bone marrow and new B cells continually to exit to the periphery at least until old age. Collectively it is believed that a common feature in the development of autoimmune disease may be dysfunctional Tregs and a consequent failure of antigen non-specific B cells to die. Thus, the compounds of the invention produce a therapeutic result by activating Tregs and killing antigen non-specific B cells.

“Autoimmune Disease” refers to those diseases which are commonly associated with the nonanaphylactic hypersensitivity reactions (Type II, Type III and/or Type IV hypersensitivity reactions) that generally result as a consequence of the subject's own humoral and/or cell-mediated immune response to one or more immunogenic substances of endogenous and/or exogenous origin. Such autoimmune diseases are distinguished from diseases associated with the anaphylactic (Type I or IgE-mediated) hypersensitivity reactions.

### (vi) Cancer

In some embodiments, the present invention provides a method of treating a to cancer comprising administering to a subject in whom such treatment is desired a therapeutically effective amount of a composition comprising a thymus derived peptide. A composition of the invention may, for example, be used as a first, second, third or fourth line cancer treatment. In some embodiments, the invention provides methods for treating a cancer (including ameliorating a symptom thereof) in a subject refractory to one or more conventional therapies for such a cancer, said methods comprising administering to said subject a therapeutically effective amount of a composition comprising a thymus derived peptide. A cancer may be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased.

Although Applicant is not bound by a specific mechanism of action it is believed that the CLIP inhibitors of the invention displace CLIP from MHC class I and cause down regulation of Treg activity and/or activation of effector T cells such as γδ T cells. Downregulation of regulatory function of Treg activity prevents suppression of the immune response and enables the subject to mount an effective or enhanced immune response against the cancer. At the same time the Treg cell may shift to an effector function, producing an antigen specific immune response. Thus, replacement of CLIP with a peptide of the invention results in the promotion of an antigen specific CD8+ response against the tumor, particularly when the peptide is administered in conjunction with a tumor specific antigen. Activation of effector T cells also enhances the immune response against the cancer, leading to a more effective treatment.

The invention provides methods for treating a cancer (including ameliorating one or more symptoms thereof) in a subject refractory to existing single agent therapies for such a cancer, said methods comprising administering to said subject a therapeutically effective amount of a composition comprising a thymus derived peptide and a therapeutically effective amount of one or more therapeutic agents other than the thymus derived peptide. The invention also provides methods for treating cancer by administering a composition comprising a thymus derived peptide in combination with any other anti-cancer treatment (e.g., radiation therapy, chemotherapy or surgery) to a patient who has proven refractory to other treatments. The invention also provides methods for the treatment of a patient having cancer and immunosuppressed by reason of having previously undergone one or more other cancer therapies. The invention also provides alternative methods for the treatment of cancer where chemotherapy, radiation therapy, hormonal therapy, and/or biological therapy/immunotherapy has proven or may prove too toxic, i.e., results in unacceptable or unbearable side effects, for the subject being treated.

Cancers that can be treated by the methods encompassed by the invention include, but are not limited to, neoplasms, malignant tumors, metastases, or any disease or disorder characterized by uncontrolled cell growth such that it would be considered cancerous. The cancer may be a primary or metastatic cancer. Specific cancers that can be treated according to the present invention include, but are not limited to, those listed below (for a review of such disorders, see Fishman et al., 1985, Medicine, 2d Ed., J.B. Lippincott Co., Philadelphia).

Cancers include, but are not limited to, biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

The compositions of the invention also can be administered to prevent to progression to a neoplastic or malignant state. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.). Hyperplasia is a form of controlled cell proliferation involving an increase in cell number in a tissue or organ, without significant alteration in structure or function. Endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

The prophylactic use of the compositions of the invention is also indicated in some viral infections that may lead to cancer. For example, human papilloma virus can lead to cervical cancer (see, e.g., Hernandez-Avila et al., Archives of Medical Research (1997) 28: 265-271), Epstein-Barr virus (EBV) can lead to lymphoma (see, e.g., Herrmann et al., J Pathol (2003) 199(2): 140-5), hepatitis B or C virus can lead to liver carcinoma (see, e.g., El-Serag, J Clin Gastroenterol (2002) 35(5 Suppl 2): S72-8), human T cell leukemia virus (HTLV)-I can lead to T-cell leukemia (see e.g., Mortreux et al., Leukemia (2003) 17(1): 26-38), and human herpesvirus-8 infection can lead to Kaposi's sarcoma (see, e.g., Kadow et al., Curr Opin Investig Drugs (2002) 3(11): 1574-9).

In other embodiments, a patient which exhibits one or more of the following predisposing factors for malignancy is treated by administration of an effective amount of a composition of the invention: a chromosomal translocation associated with a malignancy (e.g., the Philadelphia chromosome for chronic myelogenous leukemia, t(14; 18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In one set of embodiments, the invention includes a method of treating a subject susceptible to or exhibiting symptoms of cancer. The cancer may be primary, metastatic, recurrent or multi-drug resistant. In some cases, the cancer is drug-resistant or multi-drug resistant. As used herein, a “drug-resistant cancer” is a cancer that is resistant to conventional commonly-known cancer therapies. Examples of conventional cancer therapies include treatment of the cancer with agents such as methotrexate, trimetrexate, adriamycin, taxotere, doxorubicin, 5-fluorouracil, vincristine, vinblastine, pamidronate to disodium, anastrozole, exemestane, cyclophosphamide, epirubicin, toremifene, letrozole, trastuzumab, megestrol, tamoxifen, paclitaxel, docetaxel, capecitabine, goserelin acetate, etc. A “multi-drug resistant cancer” is a cancer that resists more than one type or class of cancer agents, i.e., the cancer is able to resist a first drug having a first mechanism of action, and a second drug having a second mechanism of action.

One component of the invention involves promoting an enhanced immune response against the cancer by administering the compounds of the invention. The compounds may be administered in conjunction with a cancer antigen to further promote an cancer specific immune response. A “cancer antigen” as used herein is a compound, such as a peptide or carbohydrate, associated with a tumor or cancer cell surface and which is capable of provoking an immune response when expressed on the surface of an antigen presenting cell in the context of an MHC molecule. Preferably, the antigen is expressed at the cell surface of the cancer cell. Even more preferably, the antigen is one which is not expressed by normal cells, or at least not expressed to the same level as in cancer cells. For example, some cancer antigens are normally silent (i.e., not expressed) in normal cells, some are expressed only at certain stages of differentiation and others are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. The differential expression of cancer antigens in normal and cancer cells can be exploited in order to target cancer cells. As used herein, the terms “cancer antigen” and “tumor antigen” are used interchangeably.

Cancer antigens, such as those present in cancer vaccines or those used to prepare cancer immunotherapies, can be prepared from crude cancer cell extracts, as described in Cohen, et al., 1994, Cancer Research, 54:1055, or by partially purifying the antigens, using recombinant technology, or de novo synthesis of known antigens. Cancer antigens can be used in the form of immunogenic portions of a particular antigen or in some instances a whole cell (killed) can be used as the antigen. Such antigens can be isolated or prepared recombinantly or by any other means known in the art.

Examples of cancer antigens include but are not limited to MAGE, MART-1/Melan-A, gp100, dipeptidyl peptidase IV (DPPIV), adenosine to deaminase-binding protein (ADAbp), cyclophilin b, colorectal associated antigen (CRC)—0017-1A/GA733, carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{PmeI117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2. This list is not meant to be limiting.

Another form of anti-cancer therapy involves administering an antibody specific for a cell surface antigen of, for example, a cancer cell. In one embodiment, the antibody may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA. Other antibodies include but are not limited to anti-CD20 antibodies, anti-CD40 antibodies, anti-CD19 antibodies, anti-CD22 antibodies, anti-HLA-DR antibodies, anti-CD80 antibodies, anti-CD86 antibodies, anti-CD54 antibodies, and anti-CD69 antibodies. These antibodies are available from commercial sources or may be synthesized de novo.

In one embodiment, the methods of the invention can be used in conjunction with one or more other forms of cancer treatment, for example, in conjunction with an anti-cancer agent, chemotherapy, radiotherapy, etc. (e.g., simultaneously, or as part of an overall treatment procedure). The term “cancer treatment” as used herein, may include, but is not limited to, chemotherapy, radiotherapy, adjuvant therapy, vaccination, or any combination of these methods. Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previously treatment methods. Any agent or therapy (e.g., chemotherapies, radiation therapies, surgery, hormonal therapies, and/or biological therapies/immunotherapies) which is known to be useful, or which has been used or is currently being used for the prevention or treatment of cancer can be used in combination with a composition of the invention in accordance with the invention described herein. One of ordinary skill in the medical arts can determine an appropriate treatment for a subject.

Examples of such agents (i.e., anti-cancer agents) include, but are not limited to, DNA-interactive agents including, but not limited to, the alkylating agents (e.g., nitrogen mustards, e.g. Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase II inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin Interactive agents including, but not limited to, colcbicine, Vincristine and Vinblastine, both alkaloids and Paclitaxel and cytoxan; hormonal agents including, but note limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1.alpha., IL-1 β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, IFN-β, IFN-.γ, and Uteroglobins (U.S. Pat. No. 5,696,092); anti-angiogenics including, but not limited to, agents that inhibit VEGF (e.g., other neutralizing antibodies (Kim et al., 1992; Presta et al., 1997; Sioussat et al., 1993; Kondo et al., 1993; Asano et al., 1995, U.S. Pat. No. 5,520,914), soluble receptor constructs (Kendall and Thomas, 1993; Aiello et al., 1995; Lin et al., 1998; Millauer et al., 1996), tyrosine kinase inhibitors (Siemeister et al., 1998, U.S. Pat. Nos. 5,639,757, and 5,792,771), antisense strategies, RNA aptamers and ribozymes against VEGF or VEGF receptors (Saleh et al., 1996; Cheng et al., 1996; Ke et al., 1998; Parry et al., 1999); variants of VEGF with antagonistic properties as described in WO 98/16551; compounds of other chemical classes, e.g., steroids such as the angiostatic 4,9(11)-steroids and C21-oxygenated steroids, as described in U.S. Pat. No. 5,972,922; thalidomide and related compounds, precursors, analogs, metabolites and hydrolysis products, as described in U.S. Pat. Nos. 5,712,291 and 5,593,990; Thrombospondin (TSP-1) and platelet factor 4 (PF4); interferons and metalloproteinsase inhibitors; tissue inhibitors of metalloproteinases (TIMPs); anti-Invasive Factor, retinoic acids and paclitaxel (U.S. Pat. No. 5,716,981); AGM-1470 (Ingber et al., 1990); shark cartilage extract (U.S. Pat. No. 5,618,925); anionic polyamide or polyurea oligomers (U.S. Pat. No. 5,593,664); oxindole derivatives (U.S. Pat. No. 5,576,330); estradiol derivatives (U.S. Pat. No. 5,504,074); thiazolopyrimidine derivatives (U.S. Pat. No. 5,599,813); and LM609 (U.S. Pat. No. 5,753,230); apoptosis-inducing agents including, but not limited to, bcr-abl, bcl-2 (distinct from bcl-1, cyclin D1; GenBank accession numbers M14745, X06487; U.S. Pat. Nos. 5,650,491; and 5,539,094) and family members including Bcl-x1, Mcl-1, Bak, A1, A20, and antisense nucleotide sequences (U.S. Pat. Nos. 5,650,491; 5,539,094; and 5,583,034); Immunotoxins and coaguligands, tumor vaccines, and antibodies.

Specific examples of anti-cancer agents which can be used in accordance with the methods of the invention include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; to streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The invention also encompasses administration of a composition comprising thymus derived peptide in combination with radiation therapy comprising the use of x-rays, gamma rays and other sources of radiation to destroy the cancer cells. In preferred embodiments, the radiation treatment is administered as external beam radiation or teletherapy wherein the radiation is directed from a remote source. In other preferred embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer. For instance, a patient with ovarian cancer may be administered a prophylactically or therapeutically effective amount of a composition comprising thymus derived peptide in combination with a prophylactically or therapeutically effective amount of one or more other agents useful for ovarian cancer therapy, including but not limited to, intraperitoneal radiation therapy, such as P³² therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. In a particular embodiment, a prophylactically or therapeutically effective amount of a composition of the invention is administered in combination with the administration of Taxol for patients with platinum-refractory disease. A further embodiment is the treatment of patients with refractory cancer including administration of: ifosfamide in patients with disease that is platinum-refractory, hexamethylmelamine (HMM) as salvage chemotherapy after failure of cisplatin-based combination regimens, and tamoxifen in patients with detectable levels of cytoplasmic estrogen receptor on their tumors.

Cancer therapies and their dosages, routes of administration and recommended usage are known in the art and have been described in such literature as the Physician's Desk Reference (56^{th} ed., 2002).

(vii) Alzheimer's Disease

The thymic derived peptides of the invention are also useful in treating Alzheimer's disease Alzheimer's disease is a degenerative brain disorder characterized by cognitive and noncognitive neuropsychiatric symptoms, which accounts for approximately 60% of all cases of dementia for patients over 65 years old. Psychiatric symptoms are common in Alzheimer's disease, with psychosis (hallucinations and delusions) present in many patients. It is possible that the psychotic symptoms of Alzheimer's disease involve a shift in the concentration of dopamine or acetylcholine, which may augment a dopaminergic/cholinergic balance, thereby resulting in psychotic behavior. For example, it has been proposed that an increased dopamine release may be responsible for the positive symptoms of schizophrenia. This may result in a positive disruption of the dopaminergic/cholinergic balance. In Alzheimer's disease, the reduction in cholinergic neurons effectively reduces acetylcholine release resulting in a negative disruption of the dopaminergic/cholinergic balance. Indeed, antipsychotic agents that are used to relieve psychosis of schizophrenia are also useful in alleviating psychosis in to Alzheimer's patients.

(viii) Allergic Disease

The thymic derived peptides of the invention are also useful in treating Allergic disease. A “subject having an allergic condition” shall refer to a subject that is currently experiencing or has previously experienced an allergic reaction in response to an allergen. An “allergic condition” or “allergy” refers to acquired hypersensitivity to a substance (allergen). Allergic conditions include but are not limited to eczema, allergic rhinitis or coryza, hay fever, allergic conjunctivitis, asthma, pet allergies, urticaria (hives) and food allergies, other atopic conditions including atopic dermatitis; anaphylaxis; drug allergy; and angioedema.

Allergy is typically an episodic condition associated with the production of antibodies from a particular class of immunoglobulin, IgE, against allergens. The development of an IgE-mediated response to common aeroallergens is also a factor which indicates predisposition towards the development of asthma. If an allergen encounters a specific IgE which is bound to an IgE Fc receptor (FcεR) on the surface of a basophil (circulating in the blood) or mast cell (dispersed throughout solid tissue), the cell becomes activated, resulting in the production and release of mediators such as histamine, serotonin, and lipid mediators.

An allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

Recently a role for mast cells in Treg-dependent peripheral tolerance has been suggested. Li-Fan Lu et al, Nature Mast cells are essential intermediaries in regulatory T-cell tolerance 442, 997-1002 (31 Aug. 2006). It has been proposed that the immune response to allergens in health and disease is the result of a balance between allergen-specific T_{Reg} cells and allergen-specific T_{H}2 cells. Deviation to T_{Reg} cells suppresses the production of T_{H}2-type pro-inflammatory cytokines, induces the production of allergen-specific IgG4 and IgA antibodies, and suppresses effector cells of allergy. The compounds of the invention are useful for regulating Treg activity and thus are useful in the treatment of allergy and asthma.

Symptoms of an allergic reaction vary, depending on the location within the body where the IgE reacts with the antigen. If the reaction occurs along the respiratory epithelium, the symptoms generally are sneezing, coughing and asthmatic reactions. If the interaction occurs in the digestive tract, as in the case of food allergies, abdominal pain and diarrhea are common. Systemic allergic reactions, for example following a bee sting or administration of penicillin to an allergic subject, can be severe and often life-threatening.

“Asthma” as used herein refers to an allergic disorder of the respiratory system characterized by inflammation and narrowing of the airways, and increased reactivity of the airways to inhaled agents. Symptoms of asthma include recurrent episodes of wheezing, breathlessness, chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

Asthma likely results from complex interactions among inflammatory cells, mediators, and other cells and tissues resident in the airways. Mast cells, eosinophils, epithelial cells, macrophage, and activated T cells all play an important role in the inflammatory process associated with asthma. Djukanovic R et al. (1990) Am Rev Respir Dis 142:434-457. It is believed that these cells can influence airway function through secretion of preformed and newly synthesized mediators which can act directly or indirectly on the local tissue. It has also been recognized that subpopulations of T to lymphocytes (Th2) play an important role in regulating allergic inflammation in the airway by releasing selective cytokines and establishing disease chronicity. Robinson D S et al. (1992) N Engl J Med 326:298-304.

Asthma is a complex disorder which arises at different stages in development and can be classified based on the degree of symptoms as acute, subacute, or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

A “subject having asthma” is a subject that has a disorder of the respiratory system characterized by inflammation and narrowing of the airways and increased reactivity of the airways to inhaled agents. Factors associated with initiation of asthma include, but are not limited to, allergens, cold temperature, exercise, viral infections, and SO₂.

The composition of the invention may also be administered in conjunction with an anti-allergy therapy. Conventional methods for treating or preventing allergy have involved the use of allergy medicaments or desensitization therapies. Some evolving therapies for treating or preventing allergy include the use of neutralizing anti-IgE antibodies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, corticosteroids, and prostaglandin inducers. Anti-histamines are compounds which counteract histamine released by mast cells or basophils. These compounds are well to known in the art and commonly used for the treatment of allergy. Anti-histamines include, but are not limited to, acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast. Corticosteroids include, but are not limited to, methylprednisolone, prednisolone, prednisone, beclomethasone, budesonide, dexamethasone, flunisolide, fluticasone propionate, and triamcinolone.

The composition of the invention may also be administered in conjunction with an asthma therapy. Conventional methods for treating or preventing asthma have involved the use of anti-allergy therapies (described above) and a number of other agents, including inhaled agents. Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting β₂ agonists, anti-cholinergics, and systemic corticosteroids. Asthma medicaments include, but are not limited, PDE-4 inhibitors, bronchodilator/beta-2 agonists, K+ channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors. Bronchodilator/β₂ agonists are a class of compounds which cause bronchodilation or smooth muscle relaxation. Bronchodilator/β₂ agonists include, but are not limited to, salmeterol, salbutamol, albuterol, terbutaline, D2522/formoterol, fenoterol, bitolterol, pirbuerol methylxanthines and orciprenaline

### (ix) Characterization and Demonstration of Thymus Derived Peptide Activity

The activity of the thymus derived peptides used in accordance with the present invention can be determined by any method known in the art. In one embodiment, the activity of a thymus derived peptide is determined by using various experimental animal models, including but not limited to, cancer animal models such as scid mouse model or nude mice with human tumor grafts known in the art and described in Yamanaka, 2001, Microbiol Immunol 2001; 45(7): 507-14, which is incorporated herein by reference, animal models of infectious disease or other disorders.

Various in vitro and in vivo assays that test the activities of a thymus derived peptide are used in purification processes of a thymus derived peptide. The protocols and compositions of the invention are also preferably tested in vitro, and then in vivo, for the desired therapeutic or prophylactic activity, prior to use in humans.

For instance, the thymus derived peptide may bind to CD4, gp120 or gp21, preferably in a selective manner. As used herein, the terms “selective binding” and “specific binding” are used interchangeably to refer to the ability of the peptide to bind with greater affinity to CD4, gp120 or gp21 and fragments thereof than to unrelated proteins.

Peptides can be tested for their ability to bind to CD4, gp120 or gp21 using standard binding assays known in the art. As an example of a suitable assay, CD4, gp120 or gp21 can be immobilized on a surface (such as in a well of a multi-well plate) and then contacted with a labeled peptide. The amount of peptide that binds to the CD4, gp120 or gp21 (and thus becomes itself immobilized onto the surface) may then be quantitated to determine whether a particular peptide binds to CD4, gp120 or gp21. Alternatively, the amount of peptide not bound to the surface may also be measured. In a variation of this assay, the peptide can be tested for its ability to bind directly to a CD4, gp120 or gp21-expressing cell.

Compounds for use in therapy can be tested in suitable animal model systems prior to testing in humans, including but not limited to in rats, mice, chicken, cows, monkeys, rabbits, etc. The principle animal models for cancer known in the art and widely used include, but not limited to, mice, as described in Hann et al., 2001, Curr Opin Cell Biol 2001 December; 13(6): 778-84.

In one embodiment, the S-180 cell line (ATCC CCL 8, batch F4805) is chosen as the tumor model because the same line is capable of growing both in animals and in culture (in both serum-containing and serum-free conditions). Tumors are established in mice (BALB/c) by injection of cell suspensions obtained from tissue culture. Approximately 1×10⁶ to 3×10⁶ cells are injected intra-peritoneally per mouse. The tumor developed as multiple solid nodules at multiple sites within the peritoneal cavity and to cause death in most of the animals within 10 to 15 days. In addition to monitoring animal survival, their condition is qualitatively assessed as tumor growth progressed and used to generate a tumor index as described in the following paragraph.

To establish an estimate of drug activity in tumor model experiments, an index can be developed that combines observational examination of the animals as well as their survival status. For example, mice are palpated once or twice weekly for the presence, establishment and terminal progression of the intraperitoneal S180 tumor. Tumor development and progression is assessed in these mice according to the following scale: “0”—no tumor palpated; “1”—initial tumor appears to be present; small in size (˜1 mm); no distended abdomen; “2”—tumor appears to be established; some distension of the abdomen; no apparent cachexia; “3”—tumor is well established, marked abdominal distension, animal exhibits cachexia; and, “4”—animal is dead. The index value for a treatment group is the average of the individual mouse indices in the group.

In vitro and animal models of HIV have also been described. For instance some animal models are described in McCune J. M. AIDS RESEARCH: Animal Models of HIV-1 Disease Science 19 Dec. 1997:Vol. 278. no. 5346, pp. 2141-2142 and K Uberla et al PNAS Animal model for the therapy of acquired immunodeficiency syndrome with reverse transcriptase inhibitors Aug. 29. 1995 vol. 92 no. 18 8210-8214. Uberla et al describes the development of an animal model for the therapy of the HIV-1 infection with RT inhibitors. In the study the RT of the simian immunodeficiency virus (SIV) was replaced by the RT of HIV-1. It was demonstrated that macaques infected with this SIV/HIV-1 hybrid virus developed AIDS-like symptoms and pathology. The authors concluded that “infection of macaques with the chimeric virus seems to be a valuable model to study the in vivo efficacy of new RT inhibitors, the emergence and reversal of drug resistance, the therapy of infections with drug-resistant viruses, and the efficacy of combination therapy.”

Further, any assays known to those skilled in the art can be used to evaluate the prophylactic and/or therapeutic utility of the combinatorial therapies disclosed herein for treatment or prevention of cancer and/or infectious diseases.

### (x) Dosage Regimens

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% to of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. The absolute amount will depend upon a variety of factors including the concurrent treatment, the number of doses and the individual patient parameters including age, physical condition, size and weight. These are factors well known to those of ordinary skill in the art and can be addressed with no more than routine experimentation. It is preferred generally that a maximum dose be used, that is, the highest safe dose according to sound medical judgment.

Multiple doses of the molecules of the invention are also contemplated. In some instances, when the molecules of the invention are administered with another therapeutic, for instance, an anti-HIV agent a sub-therapeutic dosage of either the molecules or the an anti-HIV agent, or a sub-therapeutic dosage of both, is used in the treatment of a subject having, or at risk of developing, HIV. When the two classes of drugs are used to together, the an anti-HIV agent may be administered in a sub-therapeutic dose to produce a desirable therapeutic result. A “sub-therapeutic dose” as used herein refers to a dosage which is less than that dosage which would produce a therapeutic result in the subject if administered in the absence of the other agent. Thus, the sub-therapeutic dose of a an anti-HIV agent is one which would not produce the desired therapeutic result in the subject in the absence of the administration of the molecules of the invention. Therapeutic doses of an anti-HIV agents are well known in the field of medicine for the treatment of HIV. These dosages have been extensively described in references such as Remington's Pharmaceutical Sciences; as well as many other medical references relied upon by the medical profession as guidance for the treatment of infectious disease, cancer, autoimmune disease, Alzheimer's disease and graft rejection. Therapeutic dosages of peptides have also been described in the art.

### (xi) Administrations, Formulations

The thymus derived peptides described herein can be used alone or in conjugates with other molecules such as detection or cytotoxic agents in the detection and treatment methods of the invention, as described in more detail herein.

Typically, one of the components usually comprises, or is coupled or conjugated to a detectable label. A detectable label is a moiety, the presence of which can be ascertained directly or indirectly. Generally, detection of the label involves an emission of energy by the label. The label can be detected directly by its ability to emit and/or absorb photons or other atomic particles of a particular wavelength (e.g., radioactivity, luminescence, optical or electron density, etc.). A label can be detected indirectly by its ability to bind, recruit and, in some cases, cleave another moiety which itself may emit or absorb light of a particular wavelength (e.g., epitope tag such as the FLAG epitope, enzyme tag such as horseradish peroxidase, etc.). An example of indirect detection is the use of a first enzyme label which cleaves a substrate into visible products. The label may be of a chemical, peptide or nucleic acid molecule nature although it is not so limited. Other detectable labels include radioactive isotopes such as P³² or H³, luminescent markers such as fluorochromes, optical or electron density markers, etc., or epitope tags such as the FLAG epitope or the HA epitope, biotin, avidin, and enzyme tags such as horseradish peroxidase, β-galactosidase, etc. The label may be bound to a peptide during or following its synthesis. There are many different labels and methods of labeling known to those of ordinary skill in the art. Examples of the types of labels that can be to used in the present invention include enzymes, radioisotopes, fluorescent compounds, colloidal metals, chemiluminescent compounds, and bioluminescent compounds. Those of ordinary skill in the art will know of other suitable labels for the peptides described herein, or will be able to ascertain such, using routine experimentation. Furthermore, the coupling or conjugation of these labels to the peptides of the invention can be performed using standard techniques common to those of ordinary skill in the art.

Another labeling technique which may result in greater sensitivity consists of coupling the molecules described herein to low molecular weight haptens. These haptens can then be specifically altered by means of a second reaction. For example, it is common to use haptens such as biotin, which reacts with avidin, or dinitrophenol, pyridoxal, or fluorescein, which can react with specific anti-hapten antibodies.

Conjugation of the peptides to a detectable label facilitates, among other things, the use of such agents in diagnostic assays. Another category of detectable labels includes diagnostic and imaging labels (generally referred to as in vivo detectable labels) such as for example magnetic resonance imaging (MRI): Gd(DOTA); for nuclear medicine: ²⁰¹T1, gamma-emitting radionuclide 99 mTc; for positron-emission tomography (PET): positron-emitting isotopes, (18)F-fluorodeoxyglucose ((18)FDG), (18)F-fluoride, copper-64, gadodiamide, and radioisotopes of Pb(II) such as 203Pb; 111In.

The conjugations or modifications described herein employ routine chemistry, which chemistry does not form a part of the invention and which chemistry is well known to those skilled in the art of chemistry. The use of protecting groups and known linkers such as mono- and hetero-bifunctional linkers are well documented in the literature and will not be repeated here.

As used herein, “conjugated” means two entities stably bound to one another by any physiochemical means. It is important that the nature of the attachment is such that it does not impair substantially the effectiveness of either entity. Keeping these parameters in mind, any covalent or non-covalent linkage known to those of ordinary skill in the art may be employed. In some embodiments, covalent linkage is preferred. Noncovalent conjugation includes hydrophobic interactions, ionic interactions, high affinity interactions such as biotin-avidin and biotin-streptavidin complexation and other affinity interactions. Such means and methods of attachment are well known to those of ordinary skill in the art.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

The conjugates also include a peptide conjugated to another peptide such as CD4, gp120 or gp21. CD4, gp120 and gp21 peptides are all known in the art.

The active agents of the invention are administered to the subject in an effective amount for treating disorders such as autoimmune disease, viral infection, bacterial infection, HIV infection, Alzheimer's disease, graft rejection, and cancer. An “effective amount”, for instance, is an amount necessary or sufficient to realize a desired biologic effect. An “effective amount for treating HIV”, for instance, could be that amount necessary to (i) prevent HIV uptake by the host cell and/or (ii) inhibit the further development of the HIV infection, i.e., arresting or slowing its development. That amount necessary for treating autoimmune disease may be an amount sufficient to prevent or inhibit a decrease in T_{H} cells compared to the levels in the absence of peptide treatment. According to some aspects of the invention, an effective amount is that amount of a compound of the invention alone or in combination with another medicament, which when combined or co-administered or administered alone, results in a therapeutic response to the disease, either in the prevention or the treatment of the disease. The biological effect may be the amelioration and or absolute elimination of symptoms resulting from the disease. In another embodiment, the biological effect is the complete abrogation of the disease, as evidenced for example, by the absence of a symptom of the disease.

The effective amount of a compound of the invention in the treatment of a disease described herein may vary depending upon the specific compound used, the mode of delivery of the compound, and whether it is used alone or in combination. The effective amount for any particular application can also vary depending on such factors as the disease being treated, the particular compound being administered, the size of the subject, or the severity of the disease or condition. One of ordinary skill in the art can empirically determine the effective amount of a particular molecule of the invention to without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

Pharmaceutical compositions of the present invention comprise an effective amount of one or more agents, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. Moreover, for animal (e.g., human) administration, it will be understood that preparations should meet sterility, pyrogenicity, general safety and purity standards as required by FDA Office of Biological Standards. The compounds are generally suitable for administration to humans. This term requires that a compound or composition be nontoxic and sufficiently pure so that no further manipulation of the compound or composition is needed prior to administration to humans.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). Except insofar as any conventional carrier is incompatible with the active ingredient, its use in the therapeutic or pharmaceutical compositions is contemplated.

The agent may comprise different types of carriers depending on whether it is to be administered in solid, liquid or aerosol form, and whether it need to be sterile for such routes of administration as injection. The present invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, to intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences (1990), incorporated herein by reference). In a particular embodiment, intraperitoneal injection is contemplated.

In any case, the composition may comprise various antioxidants to retard oxidation of one or more components. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups also can be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The composition of the invention can be used directly or can be mixed with suitable adjuvants and/or carriers. Suitable adjuvants include aluminum salt adjuvants, such as aluminum phosphate or aluminum hydroxide, calcium phosphate nanoparticles (BioSante Pharmaceuticals, Inc.), ZADAXIN™, nucleotides ppGpp and pppGpp, killed Bordetella pertussis or its components, Corenybacterium derived P40 component, cholera toxin and mycobacteria whole or parts, and ISCOMs (DeVries et al., 1988; Morein et al., 199&, Lovgren: al., 1991). The skilled artisan is familiar with carriers appropriate for pharmaceutical use or suitable for use in humans.

The following is an example of a thymus derived peptide formulation, dosage and administration schedule. The individual is administered an intramuscular or subcutaneous injection containing 8 mg of the composition (preferably 2 ml of a formulation containing 4 mg/ml of the composition in a physiologically acceptable solution) or 57 μg of thymus derived peptide per 1 kg body weight of the patient. Each treatment course consists of 16 injections; with two injections on consecutive days per week for 8 weeks. The patient's disease condition is monitored by means described below. Three months after the last injection, if the patient is still suffering from the disease, the treatment regimen is repeated. The treatment regimen may be repeated until satisfactory result is obtained, e.g. a halt or delay in the progress of the disease, an alleviation of the disease or a cure is obtained.

The composition may be formulated alone or in combination with an antigen specific for the disease state and optionally with an adjuvant. Adjuvants include for instance adjuvants that create a depo effect, immune stimulating adjuvants, and adjuvants that create a depo effect and stimulate the immune system and may be systemic or mucosal adjuvants. Adjuvants that creates a depo effect include, for instance, aluminum hydroxide, emulsion-based formulations, mineral oil, non-mineral oil, water-in-oil emulsions, oil-in-water emulsions, Seppic ISA series of Montanide adjuvants, MF-59 and PROVAX. Adjuvants that are immune stimulating adjuvants include for instance, CpG oligonucleotides, saponins, PCPP polymer, derivatives of lipopolysaccharides, MPL, MDP, t-MDP, OM-174 and Leishmania elongation factor. Adjuvants that creates a depo effect and stimulate the immune system include for instance, ISCOMS, SB-AS2, SB-AS4, non-ionic block copolymers, and SAF (Syntex Adjuvant Formulation). An example of a final formulation: 1 ml of the final composition formulation can contain: 4 mg of the composition, 0.016 M A1P0₄ (or 0.5 mg Al³⁺) 0.14 M NaCl, 0.004 M CH₃COONa, 0.004 M KCl, pH 6.2.

The composition of the invention can be administered in various ways and to different classes of recipients.

The compounds of the invention may be administered directly to a tissue. Direct tissue administration may be achieved by direct injection. The compounds may be administered once, or alternatively they may be administered in a plurality of administrations. If administered multiple times, the compounds may be administered via different routes. For example, the first (or the first few) administrations may be made directly into the affected tissue while later administrations may be systemic.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

According to the methods of the invention, the compound may be administered in a pharmaceutical composition. In general, a pharmaceutical composition comprises the compound of the invention and a pharmaceutically-acceptable carrier. Pharmaceutically-acceptable carriers for peptides, monoclonal antibodies, and antibody fragments are well-known to those of ordinary skill in the art. As used herein, a pharmaceutically-acceptable carrier means a non-toxic material that does not interfere with the effectiveness of the biological activity of the active ingredients, e.g., the ability of the peptide to bind to the target, ie HIV surface molecules.

Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compounds of the invention may be formulated into preparations in solid, semi-solid, liquid or gaseous forms such as tablets, capsules, powders, granules, ointments, solutions, depositories, inhalants and injections, and usual ways for oral, to parenteral or surgical administration. The invention also embraces pharmaceutical compositions which are formulated for local administration, such as by implants.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid to paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the active agent (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In yet other embodiments, the preferred vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the agents of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems can include time-release, delayed release or sustained release delivery systems. Such systems can avoid repeated administrations of the compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic diseases or recurrent cancer. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

Therapeutic formulations of the peptides or antibodies may be prepared for storage by mixing a peptide or antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The peptide may be administered directly to a cell or a subject, such as a human subject alone or with a suitable carrier. Alternatively, a peptide may be delivered to a cell in vitro or in vivo by delivering a nucleic acid that expresses the peptide to a cell. Various techniques may be employed for introducing nucleic acid molecules of the invention into cells, depending on whether the nucleic acid molecules are introduced in vitro or in vivo in a host. Such techniques include transfection of nucleic acid molecule-calcium phosphate precipitates, transfection of nucleic acid molecules associated with DEAE, transfection or infection with the foregoing viruses including the nucleic acid molecule of interest, liposome-mediated transfection, and the like. For certain uses, it is to preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. Especially preferred are monoclonal antibodies. Where liposomes are employed to deliver the nucleic acid molecules of the invention, proteins that bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

The peptide of the invention may also be expressed directly in mammalian cells using a mammalian expression vector. Such a vector can be delivered to the cell or subject and the peptide expressed within the cell or subject. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

As used herein, a “vector” may be any of a number of nucleic acid molecules into which a desired sequence may be inserted by restriction and ligation for expression in a host cell. Vectors are typically composed of DNA although RNA vectors are also available. Vectors include, but are not limited to, plasmids, phagemids and virus genomes. An expression vector is one into which a desired DNA sequence may be inserted by restriction and ligation such that it is operably joined to regulatory sequences and may be expressed as an RNA transcript. In some embodiments, a virus vector for delivering a nucleic acid molecule is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman Co., N.Y. (1990) and Murry, E. J. Ed. “Methods in Molecular Biology,” vol. 7, Humana Press, Inc., Clifton, N.J. (1991). In addition to delivery through the use of vectors, nucleic acids of the invention may be delivered to cells without vectors, e.g., as “naked” nucleic acid delivery using methods known to those of skill in the art.

(xii) Preparation of Peptides (Purification, Recombinant, Peptide Synthesis)

Purification Methods

The thymus derived peptides of the invention can be purified, e.g., from thymus tissue. Any techniques known in the art can be used in purifying a thymus derived peptide, including but are not limited to, separation by precipitation, separation by adsorption (e.g., column chromatography, membrane adsorbents, radial flow columns, batch adsorption, high-performance liquid chromatography, ion exchange chromatography, inorganic adsorbents, hydrophobic adsorbents, immobilized metal affinity chromatography, affinity chromatography), or separation in solution (e.g., gel filtration, electrophoresis, liquid phase partitioning, detergent partitioning, organic solvent extraction, and ultrafiltration). See Scopes, PROTEIN PURIFICATION, PRINCIPLES AND PRACTICE, 3^{rd} ed., Springer (1994), the entire text is incorporated herein by reference.

As mentioned above TNPs are typically purified from the thymus cells of freshly sacrificed, i.e., 4 hours or less after sacrifice, mammals such as monkeys, gorillas, chimpanzees, guinea pigs, cows, rabbits, dogs, mice and rats. Such methods can also be used to prepare a preparation of peptides of the invention. The nuclei from the thymus cells are isolated using methods known in the art. Part of their lysine-rich histone fractions are extracted using the pepsin degradation method of U.S. Pat. No. 4,415,553, which is hereby incorporated by reference. Other degradative methods such as trypsin degradation, papain degradation, BrCN degradation appear ineffective in extracting the thymus derived peptides. The protein rich fragment of the isolate is purified by cation exchange chromatography. For instance, the thymus derived peptides can be isolated by conducting a size exclusion procedure on an extract from the thymus of any mammal such as calf, sheep, goat, pig, etc. using standard protocols. For example, thymus extract can be obtained using the protocol of Hand et al. (1967) Biochem. BioPhys. Res. Commun. 26:18-23; Hand et al. (1970) Experientia 26:653-655; or Moudjou et al (2001) J Gen Virol 82:2017-2024. Size exclusion chromatography has been described in, for example, Folta-Stogniew and Williams (1999) 1. Biomolec. Tech. 10:51-63 and Brooks et al. (2000) Proc. Natl. Acad. Sci. 97:7064-7067. Similar methods are described in more detail in the Examples section.

The thymus derived peptides are purified from the resulting size selected protein solution via successive binding to at least one of CD4, gp 120 and gp41. Purification can be accomplished, for example, via affinity chromatography as described in Moritz et al. (1990) FEBS Lett. 275:146-50; Hecker et al. (1997) Virus Res. 49:215-223; McInerney et al. (1998) J. Virol. 72:1523-1533 and Poumbourios et al. (1992) AIDS Res. Hum. Retroviruses 8:2055-2062.

Further purification can be conducted, if necessary, to obtain a composition suitable for administration to humans. Examples of additional purification methods are hydrophobic interaction chromatography, ion exchange chromatography, mass spectrometry, isoelectric focusing, affinity chromatography, HPLC, reversed-phase chromatography and electrophoresis to name a few. These techniques are standard and well known and can be found in laboratory manuals such as Current Protocols in Molecular Biology, Ausubel et al (eds), John Wiley and Sons, New York; Protein Purification: Principles, High Resolution Methods, and Applications, 2nd ed., 1998, Janson and Ryden (eds.) Wiley-VCH; and Protein Purification Protocols, 2nd ed., 2003, Cutler (ed.) Humana Press.

Recombinant Production of the Peptides

Methods known in the art can be utilized to recombinantly produce thymus derived peptide. A nucleic acid sequence encoding thymus derived peptide can be inserted into an expression vector for propagation and expression in host cells.

An expression construct, as used herein, refers to a nucleotide sequence encoding thymus derived peptide or a fragment thereof operably associated with one or more regulatory regions which enable expression of thymus derived peptide in an appropriate host cell. “Operably-associated” refers to an association in which the regulatory regions and the thymus derived peptide sequence to be expressed are joined and positioned in such a way as to permit transcription, and ultimately, translation.

The regulatory regions necessary for transcription of the thymus derived peptide can be provided by the expression vector. In a compatible host-construct system, cellular transcriptional factors, such as RNA polymerase, will bind to the regulatory regions on the expression construct to effect transcription of the modified thymus derived peptide sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the thymus derived peptide or to insert the thymus derived peptide into the cloning site of a vector, linkers or adapters providing the appropriate compatible restriction sites may be ligated to the ends of the cDNAs by techniques well known in the art (Wu et al., 1987, Methods in Enzymol, 152: 343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising a thymus derived peptide sequence operably associated with regulatory regions can be directly introduced into appropriate host cells for expression and production of thymus derived peptide without further cloning. See, e.g., U.S. Pat. No. 5,580,859. The expression constructs can also contain DNA sequences that facilitate integration of the thymus derived peptide sequence into the genome of the host cell, e.g., via homologous recombination. In this instance, it is not necessary to employ an expression vector comprising a replication origin suitable for appropriate host cells in order to propagate and express thymus derived peptide in the host cells.

A variety of expression vectors may be used including, but not limited to, plasmids, cosmids, phage, phagemids or modified viruses. Such host-expression systems represent vehicles by which the coding sequences of interest may be produced and subsequently purified, but also represent cells which may, when transformed or transfected with the appropriate nucleotide coding sequences, express thymus derived peptide in situ. These include, but are not limited to, microorganisms such as bacteria (e.g., E. coli and B. subtilis) transformed with recombinant bacteriophage DNA, plasmid DNA or cosmid DNA expression vectors containing thymus derived peptide coding sequences; yeast (e.g., Saccharomyces, Pichia) transformed with recombinant yeast expression vectors containing thymus derived peptide coding sequences; insect cell systems infected with recombinant virus expression vectors (e.g., baculovirus) containing thymus derived peptide coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing thymus derived peptide coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli and eukaryotic cells, especially for the expression of whole recombinant thymus derived peptide molecule, are used for the expression of a recombinant thymus derived peptide molecule. For example, mammalian cells such as Chinese hamster ovary cells (CHO) can be used with a vector bearing promoter element from major intermediate early gene of cytomegalovirus for effective expression of thymus derived peptides (Foecking et al., 1986, Gene 45: 101; and Cockett et al., 1990, Bio/Technology 8: 2).

In bacterial systems, a number of expression vectors may be advantageously selected depending upon the use intended for the thymus derived peptide molecule being expressed. For example, when a large quantity of such a thymus derived peptide is to be produced, for the generation of pharmaceutical compositions of a thymus derived peptide molecule, vectors which direct the expression of high levels of fusion protein products that are readily purified may be desirable. Such vectors include, but are not limited to, the E. coli expression vector pCR2.1 TOPO (Invitrogen), in which the thymus derived peptide coding sequence may be directly ligated from PCR reaction and may be placed in frame to the lac Z coding region so that a fusion protein is produced; pIN vectors (Inouye & Inouye, 1985, Nucleic Acids Res. 13: 3101-3109; Van Heeke & Schuster, 1989, J. Biol. Chem. 24: 5503-5509) and the like. Series of vectors like pFLAG (Sigma), pMAL (NEB), and pET (Novagen) may also be used to express the foreign polypeptides as fusion proteins with FLAG peptide, malE-, or CBD-protein. These recombinant proteins may be directed into periplasmic space for correct folding and maturation. The fused part can be used for affinity purification of the expressed protein. Presence of cleavage sites for specific protease like enterokinase allows to cleave off the APR. The pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, many vectors to express foreign genes can be used, e.g., Autographa californica nuclear polyhedrosis virus (AcNPV) can be used as a vector to express foreign genes. The virus grows in cells like Spodoptera frugiperda cells. The thymus derived peptide coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the thymus derived peptide coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing thymus derived peptide in infected hosts (see, e.g., Logan & Shenk, 1984, Proc. Natl. Acad. Sci. USA 81: 355-359). Specific initiation signals may also be required for efficient translation of inserted thymus derived peptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153: 51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript and post-translational modification of the gene product, e.g., glycosylation and phosphorylation of the gene product, may be used. Such mammalian host cells include, but are not limited to, PC12, CHO, VERY, BHK, Hela, COS, MDCK, 293, 3T3, WI 38, BT483, Hs578T, HTB2, BT20 and T47D, NS0 (a murine myeloma cell line that does not endogenously produce any immunoglobulin chains), CRL7030 and HsS78Bst cells. Expression in a bacterial or yeast system can be used if post-translational modifications turn to be non-essential for the activity of thymus derived peptide.

For long term, high yield production of properly processed thymus derived peptide, stable expression in cells is preferred. Cell lines that stably express thymus derived peptide may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while thymus derived peptide is expressed continuously.

A number of selection systems may be used, including but not limited to, antibiotic resistance (markers like Neo, which confers resistance to geneticine, or G-418 (Wu and Wu, 1991, Biotherapy 3: 87-95; Tolstoshev, 1993, Ann Rev. Pharmacol. Toxicol. 32: 573-596; Mulligan, 1993, Science 260: 926-932; and Morgan and Anderson, 1993, Ann Rev. Biochem. 62: 191-217; May, 1993, TIB TECH 11 (5): 155-2 15); Zeo, for resistance to Zeocin; Bsd, for resistance to blasticidin, etc.); antimetabolite resistance (markers like Dhfr, which confers resistance to methotrexate, Wigler et al., 1980, Natl. Acad. Sci. USA 77: 357; O′Hare et al., 1981, Proc. Natl. Acad. Sci. USA 78: 1527); gpt, which confers resistance to mycophenolic acid (Mulligan & Berg, 1981, Proc. Natl. Acad. Sci. USA 78: 2072); and hygro, which confers resistance to hygromycin (Santerre et al., 1984, Gene 30: 147). In addition, mutant cell lines including, but not limited to, tk-, hgprt- or aprt-cells, can be used in combination with vectors bearing the corresponding genes for thymidine kinase, hypoxanthine, guanine- or adenine phosphoribosyltransferase. Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1.

The recombinant cells may be cultured under standard conditions of temperature, incubation time, optical density and media composition. However, conditions for growth of recombinant cells may be different from those for expression of thymus derived peptide. Modified culture conditions and media may also be used to enhance production of thymus derived peptide. Any techniques known in the art may be applied to establish the optimal conditions for producing thymus derived peptide.

Peptide Synthesis

An alternative to producing thymus derived peptide or a fragment thereof by recombinant techniques is peptide synthesis. For example, an entire thymus derived peptide, or a peptide corresponding to a portion of thymus derived peptide can be synthesized by use of a peptide synthesizer. Conventional peptide synthesis or other synthetic protocols well known in the art may be used.

Peptides having the amino acid sequence of thymus derived peptide or a portion thereof may be synthesized by solid-phase peptide synthesis using procedures similar to those described by Merrifield, 1963, J. Am. Chem. Soc., 85: 2149. During synthesis, N-α-protected amino acids having protected side chains are added stepwise to a growing polypeptide chain linked by its C-terminal and to an insoluble polymeric support, i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting thymus derived peptide or a fragment thereof is accomplished using conventional procedures, such as preparative HPLC using gel permeation, partition and/or ion exchange chromatography. The choice of appropriate matrices and buffers are well known in the art and so are not described in detail herein.

(xiii) Articles of Manufacture

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of infections, cancer, autoimmune disease, graft rejection or Alzheimer's disease.

“Instructions” can define a component of promotion, and typically involve written instructions on or associated with packaging of compositions of the invention. Instructions also can include any oral or electronic instructions provided in any manner

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The kit may have a variety of forms, such as a blister pouch, a shrink wrapped to pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. The solvent will depend on the compound and the mode of use or administration.

The kits, in one set of embodiments, may comprise a carrier means being compartmentalized to receive in close confinement one or more container means such as vials, tubes, and the like, each of the container means comprising one of the separate elements to be used in the method. For example, one of the containers may comprise a positive control for an assay. Additionally, the kit may include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

In another preferred embodiment, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses, and stored with bovine serum albumins for veterinary uses.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures (such as methods for monitoring mean absolute lymphocyte counts, tumor cell counts, and tumor size) and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising a needle or syringe, preferably packaged in sterile form, for injection of the formulation, and/or a packaged alcohol pad.

In a specific embodiment, an article of manufacture comprises packaging material and a pharmaceutical agent and instructions contained within said packaging material, wherein said pharmaceutical agent is a thymus derived peptide or a derivative, to fragment, homolog, analog thereof and a pharmaceutically acceptable carrier, and said instructions indicate a dosing regimen for preventing, treating or managing a subject with cancer, infectious disease, e.g. HIV, autoimmune disease, graft rejection, or Alzheimer's disease. In another embodiment, an article of manufacture comprises packaging material and a pharmaceutical agent and instructions contained within said packaging material, wherein said pharmaceutical agent is a thymus derived peptide or a derivative, fragment, homolog, analog thereof, a prophylactic or therapeutic agent other than a thymus derived peptide or a derivative, fragment, homolog, analog thereof, and a pharmaceutically acceptable carrier, and said instructions indicate a dosing regimen for preventing, treating or managing a subject with a cancer, infectious disease, e.g. HIV, autoimmune disease, graft rejection, or Alzheimer's disease. In another embodiment, an article of manufacture comprises packaging material and two pharmaceutical agents and instructions contained within said packaging material, wherein said first pharmaceutical agent is a thymus derived peptide or a derivative, fragment, homolog, analog thereof and a pharmaceutically acceptable carrier, and said second pharmaceutical agent is a prophylactic or therapeutic agent other than a thymus derived peptide or a derivative, fragment, homolog, analog thereof, and said instructions indicate a dosing regimen for preventing, treating or managing a subject with a cancer, infectious disease, e.g. HIV, autoimmune disease, graft rejection, or Alzheimer's disease.

(xiii) Therapeutic Monitoring

The adequacy of the treatment parameters chosen, e.g. dose, schedule, adjuvant choice and the like, is determined by taking aliquots of serum from the patient and assaying for antibody and/or T cell titers during the course of the treatment program. T cell titer may be monitored by conventional methods. For example, T lymphocytes can be detected by E-rosette formation as described in Bach, F., Contemporary Topics in Immunology, Vol. 2: Thymus Dependency, p. 189, Plenum Press, New York, 1973; Hoffman, T. & Kunkel, H. G., and Kaplan, M. E., et al., both papers are in In vitro Methods in Cell Mediated and Tumor Immunity, B. R. Bloom & R. David eds., Academic Press, New York (1976). Additionally viral load can be measured.

In addition, the clinical condition of the patient can be monitored for the desired effect, e.g. increases in T cell count and/or weight gain. If inadequate effect is achieved then the patient can be boosted with further treatment and the treatment parameters can be modified, such as by increasing the amount of the composition of the invention and/or to other active agent, or varying the route of administration.

The effect of immunotherapy with a thymus derived peptide compositions of the invention on development and progression of neoplastic diseases can be monitored by any methods known to one skilled in the art, including but not limited to measuring: a) delayed hypersensitivity as an assessment of cellular immunity; b) activity of cytolytic T-lymphocytes in vitro; c) levels of tumor specific antigens, e.g., carcinoembryonic (CEA) antigens; d) changes in the morphology of tumors using techniques such as a computed tomographic (CT) scan; e) changes in levels of putative biomarkers of risk for a particular cancer in subjects at high risk, and f) changes in the morphology of tumors using a sonogram.

Although it may not be possible to detect unique tumor antigens on all tumors, many tumors display antigens that distinguish them from normal cells. The monoclonal antibody reagents have permitted the isolation and biochemical characterization of the antigens and have been invaluable diagnostically for distinction of transformed from nontransformed cells and for definition of the cell lineage of transformed cells. The best-characterized human tumor-associated antigens are the oncofetal antigens. These antigens are expressed during embryogenesis, but are absent or very difficult to detect in normal adult tissue. The prototype antigen is carcinoembryonic antigen (CEA), a glycoprotein found on fetal gut and human colon cancer cells, but not on normal adult colon cells. Since CEA is shed from colon carcinoma cells and found in the serum, it was originally thought that the presence of this antigen in the serum could be used to screen patients for colon cancer. However, patients with other tumors, such as pancreatic and breast cancer, also have elevated serum levels of CEA. Therefore, monitoring the fall and rise of CEA levels in cancer patients undergoing therapy has proven useful for predicting tumor progression and responses to treatment.

Several other oncofetal antigens have been useful for diagnosing and monitoring human tumors, e.g., alpha-fetoprotein, an alpha-globulin normally secreted by fetal liver and yolk sac cells, is found in the serum of patients with liver and germinal cell tumors and can be used as a marker of disease status.

CT remains the choice of techniques for the accurate staging of cancers. CT has proved more sensitive and specific than any other imaging techniques for the detection of metastases.

The levels of a putative biomarker for risk of a specific cancer are measured to monitor the effect of the molecular complex of the invention. For example, in subjects at enhanced risk for prostate cancer, serum prostate-specific antigen (PSA) is measured by the procedure described by Brawer, M. K., et. al., 1992, J. Urol., 147: 841-845, and Catalona, W. J., et al., 1993, JAMA, 270: 948-958; or in subjects at risk for colorectal cancer, CEA is measured as described above in Section 5.10.3; and in subjects at enhanced risk for breast cancer, 16-hydroxylation of estradiol is measured by the procedure described by Schneider, J. et al., 1982, Proc. Natl. Acad. Sci. USA, 79: 3047-3051.

A sonogram remains an alternative choice of technique for the accurate staging of cancers.

Any adverse effects during the use of a thymus derived peptide alone or in combination with another therapy (including another therapeutic or prophylactic agent) are preferably also monitored. Examples of adverse effects of chemotherapy during a cancer treatment or treatment of an infectious disease include, but are not limited to, gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure, as well as constipation, nerve and muscle effects, temporary or permanent damage to kidneys and bladder, flu-like symptoms, fluid retention, and temporary or permanent infertility. Adverse effects from radiation therapy include, but are not limited to, fatigue, dry mouth, and loss of appetite. Other adverse effects include gastrointestinal toxicity such as, but not limited to, early and late-forming diarrhea and flatulence; nausea; vomiting; anorexia; leukopenia; anemia; neutropenia; asthenia; abdominal cramping; fever; pain; loss of body weight; dehydration; alopecia; dyspnea; insomnia; dizziness, mucositis, xerostomia, and kidney failure. Adverse effects from biological therapies/immunotherapies include, but are not limited to, rashes or swellings at the site of administration, flu-like symptoms such as fever, chills and fatigue, digestive tract problems and allergic reactions. Adverse effects from hormonal therapies include but are not limited to nausea, fertility problems, depression, loss of appetite, eye problems, headache, and weight fluctuation. Additional undesired effects typically experienced by patients are numerous and known in the art. Many are described in the Physicians' Desk Reference (56^{th} ed., 2002).

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

Thymus proteins were isolated from freshly sacrificed calf thymus according to US 20040018639. Briefly, the thymus proteins were extracted, purified and characterized from calf thymus 4 hours after sacrifice as described below.

Extraction(s) of Lysine-rich Histone Fraction from Thymus Cells with Enzyme Degradation by Pepsin: The thymus tissue and its associated connective tissues were separated from a calf within 4 hours of its sacrifice. The tissues were washed with a solution containing 0.14 M NaCl, and 0.005 M EDTA-Na₃ at 4° C. for 5 minutes. The wash solution was decanted and the tissues were washed a second time under the same conditions. After decantation of the wash solution, the tissues were weighed. The tissues were homogenized in 0.14 M NaCl, 0.005 M KCl, 0.005 M MgCl₂, 0.003 M CaCl₂, 0.15 M TRIS-HCl, pH 7.6, 0.25 M sucrose in a tissue homogenizes (Brinkman Polytron Homogenizes, Brinkman Instruments, Inc., Westbury, N.Y.), at 4° C. and at an rpm and for the duration of time recommended by the manufacturer of the homogenizer for removal of cell nuclei. The ratio of the tissue to the buffer was 1:4 (weight/weight).

The tissue homogenate was then filtered through gauze pad by vacuum. The filtrate was centrifuged at 1,000 g at 4° C. for 90 minutes. The supernatant was discarded. The pellet was resuspended in 0.008 NaCl, 0.003 M CaCl₂, 0.003 M MgCl₂, 0.08 M NaH₂P0₄, 0.002 M TRIS-HCl, 0.25 M sucrose, pH 5.2. The ratio of the pellet to the buffer was 1:4 (weight/weight). The resuspension was homogenated in a beaker with a magnetic stirrer at 200 rpm, at 4° C. for 5 minutes. The homogenate was then centrifuged at 3500 g, at 4° C., for 60 minutes. The supernatant was discarded. The pellet was resuspended in 0.014 M NaCl, 0.001 M CaCl, 0.002 M MgCl₂, 0.001 M EDTA-Na₃, 0.002 M TRIS-HCl, 0.25 M sucrose, pH 4.2 at a ratio of pellet to buffer of 1:4 (weight/weight). The resuspension was homogenated in a beaker with a magnetic stirrer at 200 rpm, at 4° C. for 5 minutes. The homogenate was then centrifuged at 8000 g, at 4° C. for 60 minutes. The supernatant was discarded.

The pellet was resuspended at a ratio of 1:4 (weight/weight) with a previously prepared buffer containing: 1 part volume/volume) Solution 1, 2 parts Solution 2, and 17 parts Buffer 4. Solution 1 consisted of: 10% sodium dodecyl sulfate in water/ethanol (at 55:45 v/v). Solution 2 consisted of 10% Tween 80 (in distilled water). Buffer 4 consisted of: 0.011 M NaH₂P0₄ and 0.19 M Na₂HP0₄, pH 7.4.

The resuspension was homogenated in a beaker with a magnetic stirrer at 200 rpm, at 4° C. for 15 minutes. The homogenate was then centrifuged at 12,000 g, at 4° C. for 60 minutes. The supernatant was discarded. The pellet was weighed and resuspended in 0.05 M Na₃C₆H₅0₇, 0.05 M CH₃COONa, 0.1 N HCl, pH 2.8 at a ratio of pellet to buffer of 1:4 (weight/weight). The resuspension was homogenized by tissue homogenizer at 1000 rpm, at 4° C. for 1 minute.

Pepsin (catalog number P 7000, Sigma Chemical Company, St. Louis, Mo.) diluted in distilled water at 1:10,000, with activity of 800-2500 units per mg protein, was added to the homogenate at a pepsin (powder) to pellet after homogenization weight ratio of 100:1.8. The mixture was placed in a beaker and stirred, under nitrogen atmosphere, with a magnetic stirrer at 45 rpm, at 4° C. for 12 hours.

The resulting mixture was then centrifuged at 12,000 g, at 4° C. for 60 minutes. The pellet was discarded. The supernatant was removed and precipitated with a solution consisting of saturated (NH₄)₂ S0₄. One part of the supernatant was mixed with one part of the solution and stirred with a magnetic stirrer at 600 rpm for 6 hours at 4° C. The mixture was then centrifuged at 12,000 g, at 4° C. G for 60 minutes. The supernatant was discarded. The pellet was dissolved in a solution containing a minimal quantity of 0.1 M NaCl, 0.1 M CH₃COONa, 0.02 M thiodiglycol. The resulting solution was dialyzed against 0.01 M NaCl, 0.01 M CH₃COONa, pH 6:4 until the ammonium sulfate was removed from the dialysate.

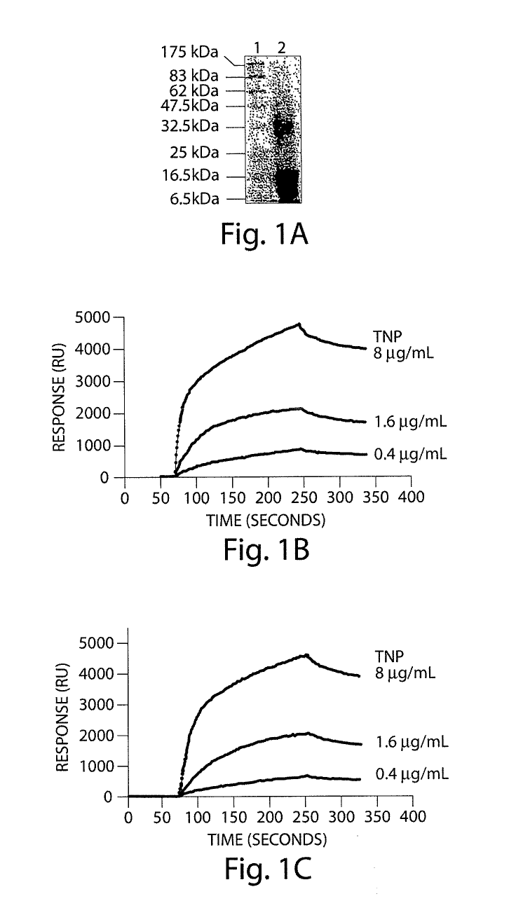

The protein concentration was determined by the Bradford assay with bovine serum albumin (Sigma, Cat. No A-3912) as the calibration standard. The purity of the samples was analyzed by SDS-Polyacrylamide gel electrophoresis (SDS-PAGE) using 10% and/or 15% polyacrylamide gels. The resolved proteins were visualized by Coomassie brilliant blue-R250 and/or Silver Staining (BioRad, Cat #161-0443) to according to the manufacturer's protocol. Molecular weights of proteins bands were estimated by comparing their relative mobility to those of marker proteins of known molecular weights (BioRad, Cat. #161-0314), run on the same gel (FIG. 1A).

Binding studies were performed on the BIAcore 2000 (Biacore, Sweden). Recombinant human CD4 (Progenies, Cat. # PRO 1008-1), recombinant HIV-1 gp120 (NIH AIDS Research & Reference Reagent Program, #4961) and gp41 (546-682 aa) were immobilized to the surface of biosensor chip (CM5) via an amine coupling of the appropriate protein to carboxyl groups in the dextran matrix of the chip. Serial dilutions of the crude sample in the running buffer containing 10 mM HEPES, 150 mM NaCl, 0.05% surfactant P20, pH 7.4 were injected at 5 μl/min over each immobilized target and the kinetics of binding/dissociation was measured as change of the SPR signal (in resonance units—RU). Each injection was followed by a regeneration step of 30-sec pulse of 1M NaCl, 50 mM NaOH. Fitting of experimental data was done with BIAevaluation 3.0 software. The crude protein strongly bound to CD4 molecules (FIG. 1B) and to gp 41 and gp 120 of HIV-1 (FIGS. 1C and 1D, respectively), but not to BSA.

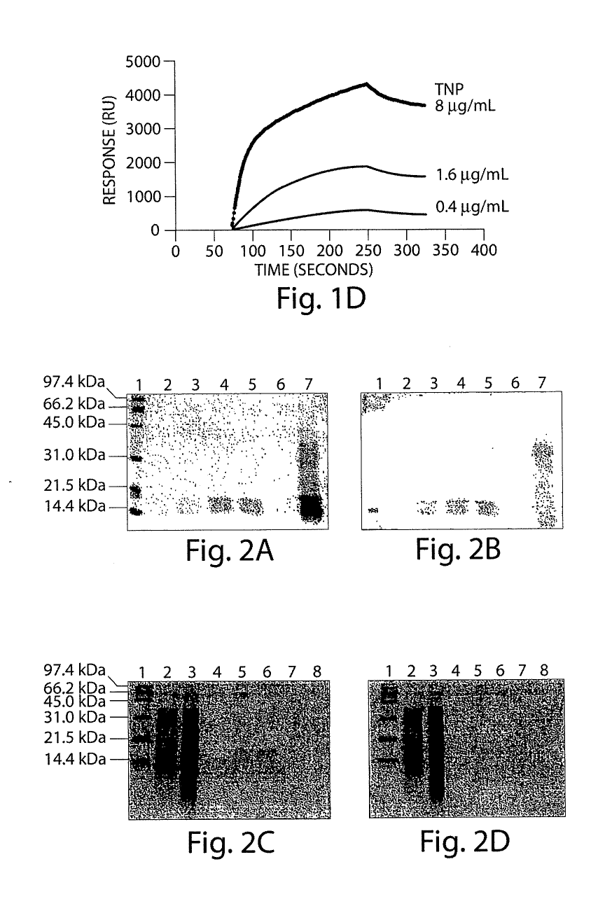

Protein fractions from the isolated thymus protein sample were purified using an affinity chromatography column (MicroLink™ Protein Coupling Kit, Pierce, Cat. #20475) according to the manufacture's instructions. Briefly, 0.2 mg of recombinant human CD4 (Progenies, Cat. # PRO 1008-1) or recombinant HIV-1 gp 120 (NIH AIDS Research & Reference Reagent Program, #4961), or irrelevant antigen (amyloid beta peptide) were immobilized on an AminoLink coupling gel and the remaining active binding sites were blocked with 1M Tris.HCl, 0.05% NaN₃. 1 mL of crude thymus protein sample was incubated with the immobilized protein to form an immune complex. The gel-bound complex was then washed to remove irrelevant material. Proteins specifically bound to CD4 or gp 120 were eluted with primary amines containing solution (pH 2.8) and neutralized. Eluted fractions were analyzed by 15% SDS-PAGE followed by Coomassie brilliant blue-R250 and/or silver staining (FIGS. 2A and 2B, respectively) and the concentration was determined by Bradford protein assay. Molecular sizes of these bands were around 14-17 kDa.

Specificity of these proteins was confirmed by purifying the same sample using two amino link columns, one coupled with gp120 and another one with human amyloid beta peptide, and running different fractions of the eluted proteins on a 15% SDS-PAGE gel. Three slender bands were detected representing low molecular weight proteins specific to gp120 in fractions #2, #3, and #4 eluted from the column with gp120 (FIG. 2C), while no protein was found in any fractions eluted from the column with amyloid beta protein (FIG. 2D). Fractions #2-4 eluted from the gp120 column were passed through another amino link column coupled with CD4. All three proteins that bound to gp120 were also specific to CD4 molecules, and 14-17 kDa bands detected in a 15% SDS-PAGE gel (FIG. 2E).

### Example 2

Sequence analysis of the three bands with approximate molecular weights of 16,000; 15,000 and 12,000 Daltons was performed at the Molecular Structure Facility at the University of California, Davis by de novo sequencing using tandem mass spectrometry. Protein analysis was performed using a Finnigan LCQ Deca XP Plus (San Jose, Calif.) coupled directly to an LC column. The Sequest analysis software (Bioworks v. 3.1) was used to identify the peptide sequences in a human or bovine protein database that best match the observed MS/MS spectra.

The results from the bovine database identified the 16 kDa protein as histone H 1.1 or H2B. Analysis also indicates that the 15 kDa and 12 kDA proteins likely represent bovine H1.1 sequence (50.5% and 48.6% sequence coverage, respectively). In addition to these analyses the sequences were also compared to the human database. Again, the 16 kDa protein likely represents human histone H2.B (42.1% coverage), although the sequence of this protein has 24.5% identity with amino aid sequence of human Cystatin A as well. Interestingly, the 15 kDa protein also showed 42.9% identity to cystatin A while the 12 kDa protein showed 6 1.2% identity. Of note, these molecules also had about 24% identical amino acids sequences with HI histone family.

### Example 3

The identity of histones and cystatin A was confirmed by directly demonstrating to binding of these proteins to HIV1 gp120 and human CD4 molecules. Binding studies were performed on the BIAcore 2000 (Biacore, Sweden). Recombinant human CD4 (Progenies, Cat. # PRO 1008-1), recombinant HIV-1 gp120 (NIH AIDS Research & Reference Reagent Program, #4961) and gp41 (546-682 aa) were immobilized to the surface of biosensor chip (CM5) via an amine coupling of the appropriate protein to carboxyl groups in the dextran matrix of the chip. Serial dilutions of the crude sample in the running buffer containing 10 mM HEPES, 150 mM NaCl, 0.05% surfactant P20, pH 7.4 were injected at 5 μl/min over each immobilized target and the kinetics of binding/dissociation was measured as change of the SPR signal (in resonance units RU). Each injection was followed by a regeneration step of 30-sec pulse of 1M NaCl, 50 mM NaOH. Fitting of experimental data was done with BIAevaluation 3.0 software.

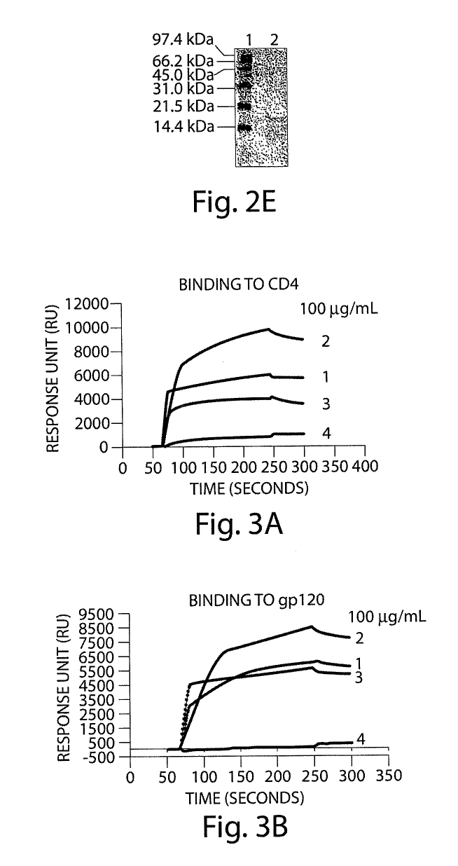

Four out of five histones bound to gp120 and CD4 molecules very well (FIGS. 3A and B). However, the affinity of binding to gp120 was significantly higher than that for CD4.

### Example 4

The binding affinity of the cystatin A and histone components of the composition are determined using any standard protocol, such as isothermal titration calorimetry (Velazquez-Campoy and Freire (2006) Nature Protocols 1: 186-191; Sigurskjold (2000) Anal Biochem 277:260-266; Wiseman et al. (1989) Anal. Biochem 179: 131-137; which are incorporated in their entirety by reference). Alternatively, the binding affinities are determined using Biacore technology.

The following Examples 5-12 are reproduced from U.S. Ser. No. 12/011,643 filed on Jan. 28, 2008, naming Karen Newell, Evan Newell and Joshua Hunter Cabrera as inventors. It is included here solely to provide a background context to the invention. The experiments reflect the invention of an overlapping but different inventive entity than is named on the instant application.

### Example 5

### B-Cell Apoptosis after Coxsackievirus Infection

During the course of Coxsackievirus infection, animals that recover from the virus without subsequent autoimmune sequelae have high percentages of splenic B cell apoptosis during the infection in vivo. Those animals susceptible to Coxsackievirus-mediated autoimmune disease have non-specifically activated B cells that do not undergo apoptosis, at least not during acute infection, nor during the time period prior to autoimmune symptoms indicating that a common feature in the development of autoimmune disease is failure of non-specifically activated B cells to die.

### Example 6

### Activated B Cells in HIV Disease Mediate NK Cell Activation

Polyclonal activation of peripheral blood human B cells is experimentally induced in an antigen-independent fashion using a combination of CD40 engagement (CD40 Ligand bearing fibroblasts) and culture in recombinant IL-4. The activated B cells are isolated and returned to co-culture with autologous peripheral blood mononuclear cells (PBMCs). After five days of co-culture, a striking increase in the percentage of activated NK cells in the PBMC culture (NK cells accounting for up to 25-50%, of the surviving PBMCs) was observed. A dramatic apoptotic loss of the activated B cells was also observed. These data indicate that antigen-independent activated B cells in HIV disease initially activate NK cells.

### Example 7

### Antigen-Independent B Cell Activation Results in NK Cell Activity

Elements of HIV infection that provide an antigen-independent activation signal to B cells that results in NK cell activation and polyclonal B cell activation are examined.

Antigen-independent activation of B cells: Human B cells: PBMCs are prepared from 5 normal and 5 HIV-infected adult donors using standard Ficoll-Hypaque density-gradient techniques. Irradiated (75 Gy) human CD40L-transfected murine fibroblasts (LTK-CD40L), are plated in six-well plates (BD Bioscience, Franklin Lakes, N.J.) at a concentration of 0.1×106 cells/well, in RPMI complete medium and cultured overnight at 37° C., 5% CO2. After washing twice with PBS, 2×106 cells/mL PBMC are co-cultured with LTK-CD40L cells in the presence of recombinant human interleukin-4 (rhIL-4; 4 ng/mL; Peprotech, Rocky Hill, N.J.) or with purified HIV derived gp 120 protein in complete Dulbecco's medium (Invitrogen), supplemented with 10% human AB serum (Gemini Bio-Product, Woodland, Calif.) Cultured cells are transferred to new plates with freshly prepared, irradiated LTK-CD40L cells every 3 to 5 days. Before use, dead cells are removed from the CD40-B cells by Ficoll density centrifugation, followed by washing twice with PBS. The viability of this fraction is expected to be >99%, and >95% of the cells, using this protocol, have been shown to be B cells that are more than 95% pure CD19+ and CD20+ after 2 weeks of culture. This protocol yields a viability of >99%, and >95% of the cells have been shown to be B cells that are more than 95% pure CD19+ and CD20+ after 2 weeks of culture.

The activated B cells are co-cultured with autologous PBMC at a ratio of 1:10 and cultured for five days. Harvested cells are stained with fluorochrome-conjugated antibodies (BD Pharmingen) to CD56, CD3, CD19, CD4, and CD8. Cells are analyzed flow cytometrically to determine the percentage of NK cells (Percent CD56+, CD3−) resulting from co-culture comparing non-infected to infected samples. NK cells are counter-stained for NK killing ligand KIR3DS1, NKG2D, FaL, or PD1. Similarly the percent surviving large and small C19+ cells are quantitated flow cytometrically.

B cell activation in HIV: To determine if activated NK or CD3 T cells promote polyclonal B cell activation, reciprocal co-culture experiments are performed in which NKs or CD3+ T cells are activated and co-cultured 1:10 in PBMC from the autologous donors. PBMCs are prepared from HIV infected or uninfected adult donors using standard Ficoll-Hypaque density-gradient techniques. To activate NKs and CD3+ T cells, PBMCs are cultured in RPMI with 10% FCS, 1 mM penicillin, 1 mM Glutamax, and 1% W/V glucose at 2.0-4.0×106/mL for 3 days with 1:40,000 OKT3, 100 U/mL IL-2, or no stimulation (resting). After 3 days stimulation, non-adherent PBMCs are gently harvested and immune cell subsets are purified by MACS technology according to manufacturers protocol (Miltenyi Biotec, Auburn Calif.). In brief, NK cells are first selected using the CD56+multisort kit, followed by bead release, and depletion with anti-CD3 beads. T cells are obtained by depleting non-adherent PBMCs with CD56 beads with or without anti-CD4 or anti-CD8 beads for isolation of each individual subset. Purity of cell fractions are confirmed for each experiment by flow cytometry using CD56, CD3, CD4, CD8 and CD14 antibodies. Following culture for 5 days, flow cytometry is used to determine relative changes in CD19+, CD4, CD8, NK, CD3, and CD69 as a marker for activation.

The NK cells from the co-culture experiments for KIR3DS1 and other killer cell to ligands including NKG2D ligand, PD1, and FasL that are indicative of killer cell functions are examined.

Antigen-independent activation of mouse B cells. Mouse spleens are removed from C57B16 mice, red cells are removed using buffered ammonium chloride, T cells are depleted with an anti-T cell antibody cocktail (HO13, GK1.5 and 30H12) and complement. T depleted splenocytes are washed and fractionated using Percoll density gradient centrifugation. The B cells are isolated at the 1.079/1.085 g/ml density interface (resting B cells) and washed to remove residual Percoll. The cells are cultured in the presence of LPS or tri-palmitoyl-S-glyceryl-cysteinyl N-terminus (Pam(3)Cys), agonists of TLR2, on B cells. The activated B cells are co-cultured with total spleen cells at a ratio of 1:10 B cell:total spleen cells. After five days in culture, the remaining cells are analyzed for expansion of cell subsets including those expressing mouse CD56, CD3, B220, CD4 and CD8. These cell surface molecules are analyzed flow cytometrically. CD56+CD3− cells are counterstained for NKG2D and other death-inducing receptors.

### Example 8

### NK Cells Kill Activated CD4+ T Cells

The ability of NK cells to lyse activated CD4 T cells as targets as a result of NK cell activation and changes in the CD4 T cell target is examined.

Activation of Human NK and CD3+ T cells: PBMCs are prepared from HIV infected or uninfected adult donors using standard Ficoll-Hypaque density-gradient techniques. NKs and CD3+ T cells are activated and isolated as disclosed herein. T cells and NK cells are routinely between 80-95% pure with less than 1% monocyte contamination. T cell activation in OKT3-stimulated PBMCs is confirmed by assays using 3H-thymidine incorporation. NK cell activation is confirmed by increase in size and granularity by flow cytometry, by staining for CD56+ and CD3− flow cytometrically, and by lytic activity as measured by chromium release of well-established NK targets. We load well-established NK cell targets or the non-specifically activated B cells as disclosed herein with 51-Chromium. Chromium release is used as a measurement of target cell death.

Activation of mouse NK and CD3+ T cells: Splenocytes are isolated as disclosed herein. The red blood cell-depleted spleen cells are cultured in recombinant mouse IL-2 or with 145.2C11 (anti-mouse CD3, Pharmingen) for 3 days. After stimulation, the cells are harvested and purified using Cell-ect Isolation kits for either NK, CD4, or CD8+ T cells. The cells are then co-cultured with 51-Chromium-labelled, well-established NK cell targets or with 51-Chromium-labelled non-specifically activated B cells as disclosed herein.

### Example 9

### Chronically Activated HIV Infected (or HIV-Specific CD4 T Cells) are the Intercellular Targets of Activated Killer Cells

Chronically activated CD4+ T cells become particularly susceptible to killer cells as a consequence of the chronic immune stimulation resulting from HIV infection.

NK cells are isolated from uninfected or HIV-infected individuals using the CD56+multisort kit as disclosed herein. The cells are activated in IL-2 as disclosed herein. Co-culture experiments are performed with these cells added back to PBMC at a 1:10 ratio from autologous donors. Prior to co-culture the NK cells from HIV infected and uninfected donors are examined for death-inducing receptor: ligand pairs killer, including KIR3DS1, FasL, and NKG2D ligands that are indicative of killer cell functions. In parallel, pre- and post-coculture PBMCs from the autologous donors of HIV infected or uninfected donors are stained.

### Example 10

### TNP MIXTURE Displaces CLIP from Model B Cell Lines

Kinetics of CLIP displacement from the surface of model B cells lines (Daudi and Raji) in response to thymic nuclear protein mixture was determined.

Results were expressed in histogram analyses. The Y axis represents cell number of the 5000 live cells versus the X axis which is a reflection of relative Fitc fluorescence. The distance between the histogram from the isotype control staining versus the histogram reflecting the specific stain is a measure of level of cell surface CLIP on a population of live Raji or Daudi cells as indicated.

At three hours, on both cell lines, evidence was observed by diminished ratio of Isotype to CLIP staining, that the TNP mixtures at 200 microgram/ml cause a reduction in detectable cell surface CLIP.

At 24 hours, the effect was less, and may have caused an increase in detectable CLIP. Noticeably at 24 hours, the TNP mixture caused death of the B cell lines at the 200 microgram/mL concentrations and by 48 hours all of the cells treated with 200 micrograms were dead and the 50 microgram concentrations also resulted in significant toxicity.

At 3 hours, treatment with 200 micrograms TNP/ml, there was 2.5 times the number of dead cells as determined by Trypan blue exclusion. Cell death in the flow cytometric experiments was, determined by forward versus side scatter changes (decreased forward scatter, increased side scatter).

Materials and Methods

Cell Culture Conditions: The Raji and Daudi cell lines were purchased from American Type Culture Collection, were thawed, and grown in RPMI 1640 medium supplemented with standard supplements, including 10% fetal calf serum, gentamycin, penicillin, streptomycin, sodium pyruvate, HEPES buffer, 1-glutamine, and 2-ME.

Protocol: Cells were plated into a 12 well plate with 3 mls total volume containing approximately 0.5×106/well for Daudi cells and 1.0×106/well for Raji cells. Treatment groups included no treatment as control; 50 micrograms/ml TNP mixture; 200-micrograms/ml TNP mixture; 50 micrograms of control bovine albumin; or 200 micrograms/ml bovine albumin as protein controls.

The cells were incubated at 37° C. in an atmosphere containing 5% CO2 and approximately 92% humidity. The cells were incubated for 3, 24, and 48 hours. At each time point, the cells from that experimental time were harvested and stained for flow cytometric analysis of cell surface expression of CLIP (MHC Class II invariant peptide, human) by using the commercially available (Becton/Dickinson/Pharmingen) anti-human CLIP Fitc. Catalogue #555981.

Harvested cells were stained using standard staining procedure that called for a 1:100 dilution of Fitc-anti-human CLIP or isotype control. Following staining on ice for 25 minutes, cells were washed with PBS/FCS and resuspended in 100 microliters and added to staining tubes containing 400 microliters of PBS. Samples were acquired and analyzed on a Coulter Excel Flow Cytometer.

### Example 11

### MKN1 (bioCLIP) Alters Cell Surface CLIP and CD74 Levels

The ability of MKN1 (bioCLIP) to alter cell surface CLIP and CD74 levels was determined using Raji or Daudi cells.

Data were analyzed by histogram with Y axis represents cell number of the 5000 live cells versus the X axis which is a reflection of relative FITC fluorescence with either antibodies to CLIP or CD74. The distance between the histogram from the isotype control staining versus the histogram reflecting the specific stain and is a measure of level of cell surface CLIP or CD74 when staining a population of live Raji or Daudi cells.

The results show that treatment with MKN1 (bioCLIP) alters cell surface CLIP and CD74 levels.

Materials and Methods:

Cell Culture Conditions: The Raji and Daudi cell lines were purchased from American Type Culture Collection, were thawed, and grown in RPMI 1640 medium supplemented with standard supplements, including 10% fetal calf serum, gentamycin, penicillin, streptomycin, sodium pyruvate, HEPES buffer, 1-glutamine, and 2-ME.

Protocol: Cells were plated into a 12 well plate with 3 mls total volume containing approximately 0.5×106/mL for Daudi cells and 0.5×106/mL for Raji cells. Treatment groups included no treatment as control; MKN 3 and MKN 5 at 50 microMolar final concentration based on the reported molarity of the synthesized compounds.

Peptide 1: MKN.1 (19 mer) Biotin at N-Terminal=Biotinylated CLIP

SGG GSK MRM ATP LLM QAL Y (SEQ ID NO 266)

5-10 mg Obtained @>95% purity (ELIM Pharmaceuticals)

The cells were incubated at 37° C. in an atmosphere containing 5% CO2 and approximately 92% humidity. The cells were incubated for 24 and 48 hours. At each time point, the cells from that experimental time were harvested and stained for flow cytometric analysis of cell surface expression of CLIP (MHC Class II invariant peptide, human) by using the commercially available (Becton/Dickinson/Pharmingen) anti-human CLIP Fitc. Catalogue #555981 versus Streptavidin and for CD74 using the commercially available (Becton/Dickinson/Pharmingen) anti-human CC74 Fitc antibody.

Harvested cells were stained using standard staining procedure that called for a 1:100 dilution of Fitc-anti-human CLIP or CD74 antibody (Fitc, Pharmingen, Cat #554647) or isotype control. Following staining on ice for 25 minutes, cells were washed with PBS/FCS and resuspended in 100 microliters and added to staining tubes containing 400 microliters of PBS. Samples were acquired and analyzed on a Coulter Excel Flow Cytometer.

### Example 12

### 2-Deoxyglucose and Dichloroacetate Cause Removal of B Cell Surface CLIP

The ability of 2-Deoxyglucose and dichloroacetate affect B cell surface CLIP was determined. The results show that treatment equimolar amounts of 2-deoxyglucose and dichloroacetate decrease (remove) cell surface CLIP from both B cell lines optimally at 48 hours.

Materials and Methods

Cell Culture Conditions: The Raji and Daudi cell lines were purchased from American Type Culture Collection, were thawed, and grown in RPMI 1640 medium supplemented with standard supplements, including 10% fetal calf serum, gentamycin, penicillin, streptomycin, sodium pyruvate, HEPES buffer, 1-glutamine, and 2-ME.

Protocol: Cells were plated into a 12 well plate with 3 mls total volume containing approximately 0.5×106/ml for Daudi cells and 0.5×106/ml for Raji cells. Treatment groups included no treatment as control; MKN 3 and MKN 5 at 50 microMolar final concentration based on the reported molarity of the synthesized compounds.

The cells were incubated at 37° C. in an atmosphere containing 5% CO2 and approximately 92% humidity. The cells were incubated for 4, 24 and 48 hours with or without 2 deoxyglucose and dichloroacetate at 1 mg/ml of each compound. At each time point, the cells from that experimental time were harvested and stained for flow cytometric analysis of cell surface expression of CLIP (MHC Class II invariant peptide, human) by using the commercially available (Becton/Dickinson/PHarmingen) anti-human CLIP Fitc. Catalogue #555981.

Harvested cells were stained using standard staining procedure that called for a 1:100 dilution of Fitc-anti-human CLIP (Fitc, Pharmingen, Cat #555981) or isotype control. Following staining on ice for 25 minutes, cells were washed with PBS/FCS and resuspended in 100 microliters and added to staining tubes containing 400 microliters of PBS. Samples were acquired and analyzed on a Coulter Excel Flow Cytometer.

The following Example 13 is included in a co-pending application filed concurrently herewith entitled “CLIP inhibitors and methods of modulating immune function” and naming the instant inventors. The data was generated under the direction to of Karen Newell.

### Example 13

### Prediction of the Sequence of Bio-Active Peptides that have a High Affinity for the Majority of the HLA-DR, DP, and DQ Alleles

Based on a computational model comparing the peptide content of TNP mixture and identifying those peptides that would have the likeliest ability to compete for the peptide/antigen binding site for MHC class II (human HLA-DR, DP, and DQ), several peptide candidates were synthesized and examined for activity. The purpose of the study was to determine if synthetic peptides can compete for binding with CLIP peptides as measured with either Fitc anti-human CLIP antibody or, comparatively in the case of biotinylated peptides, with Streptavidin.

Materials and Methods

Cell Culture Conditions: The Raji and Daudi cell lines were purchased from American Type Culture Collection, were thawed, and grown in RPMI 1640 medium supplemented with standard supplements, including 10% fetal calf serum, gentamycin, penicillin, streptomycin, sodium pyruvate, HEPES buffer, 1-glutamine, and 2-ME.

Protocol: Cells were plated into a 12 well plate with 3 mls total volume containing approximately 1.5×10⁶/well for Daudi cells and 3.0×10⁶/well for Raji cells. Treatment groups included no treatment as control; MKN 3 and MKN 5 at 50 microMolar final concentration based on the reported molarity of the synthesized compounds.

The following peptides were synthesized by ELIM Pharmaceuticals.

Peptide 1: MKN.1 (19 mer) Biotin at N-Terminal =
Biotinylated CLIP

| | |
|---|---|
| SGG GSK MRM ATP LLM QAL Y | (SEQ ID NO 268) |
| 5-10 mg @ >95% purity | |

Peptide 2: MKN.2 (15 mer) No modification = Cold
CLIP

| | |
|---|---|
| SKM RMA TPL LMQ ALY | (SEQ ID NO 267) |
| 5-10 mg @ >95% purity | |

Peptide 3: MKN.3 (21 mer) Biotin at N-Terminal =
Biotinylated FRIMAVLAS

| | |
|---|---|
| SGG GAN SGF RIM AVL ASG GQY | (SEQ ID NO 268) |
| 5-10 mg @ >95% purity | |

Peptide 4: MKN.4 (17 mer) No modification = Cold
FRIMAVLAS

| | |
|---|---|
| ANS GFR IMA VLA SGG QY | (SEQ ID NO 269) |
| 5-10 mg @ >95% purity | |

Peptide 5: MKN.5 (18 mer) Biotin at N-Terminal =
Biotinylated TNP1

| | |
|---|---|
| SGG GKA LVQ NDT LLQ VKG | (SEQ ID NO 270) |
| 5-10 mg @ >95% purity | |

Peptide 6: MKN.6 (14 mer) No modification = TNP1

| | |
|---|---|
| KAL VQN DTL LQV KG | (SEQ ID NO 1) |
| 5-10 mg @ >95% purity | |

The cells were incubated at 37° C. in an atmosphere containing 5% CO₂ and approximately 92% humidity. The cells were incubated for 4 and 24 hours. At each time point, the cells from that experimental time were harvested and stained for flow cytometric analysis of cell surface expression of CLIP (MHC Class II invariant peptide, human) by using the commercially available (Becton/Dickinson/PHarmingen) anti-human CLIP Fitc. Catalogue #555981 versus Streptavidin.

Harvested cells were stained using standard staining procedure that called for a 1:100 dilution of Fitc-anti-human CLIP or isotype control versus 1:200 dilution of the commercially prepared Streptavidin. Following staining on ice for 25 minutes, cells were washed with PBS/FCS and resuspended in 100 microliters and added to staining tubes containing 400 microliters of PBS. Samples were acquired and analyzed on a Coulter Excel Flow Cytometer.

Results:

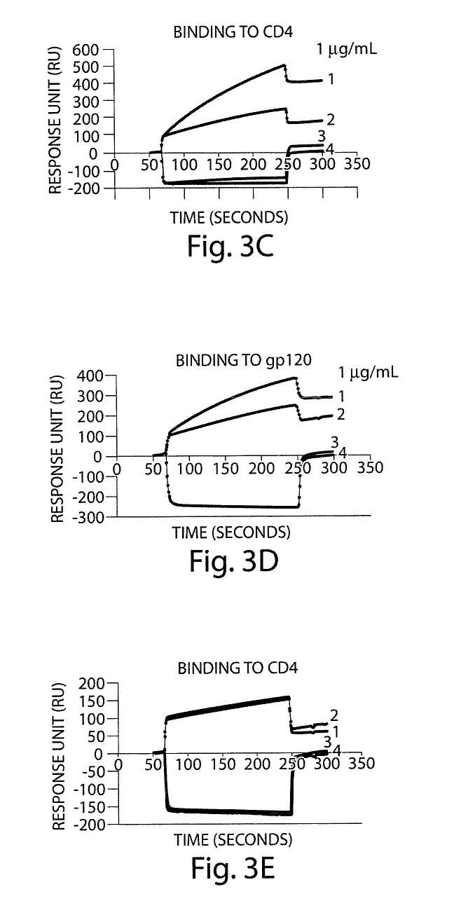

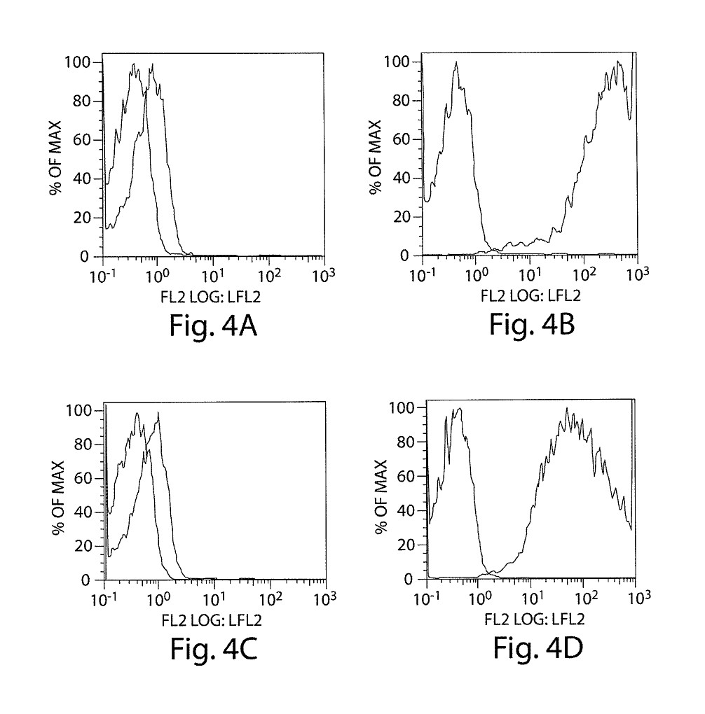

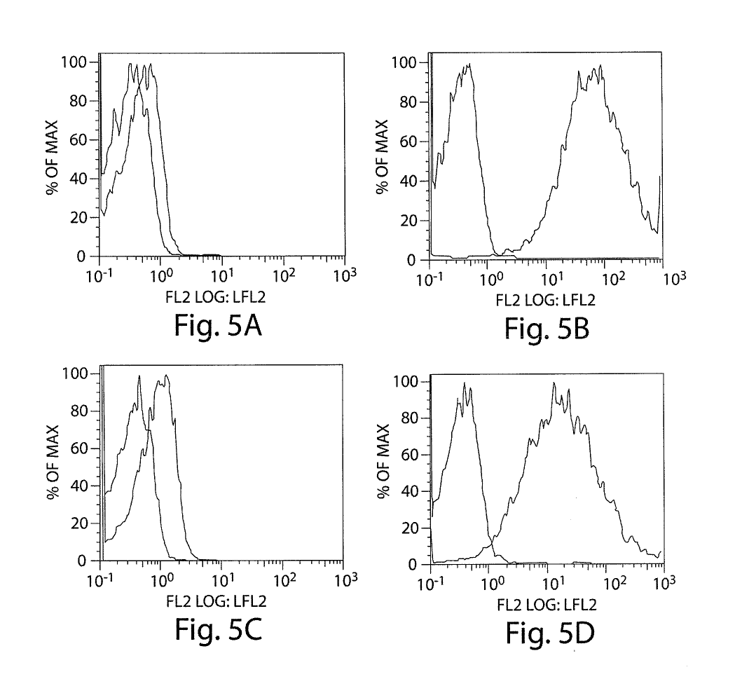

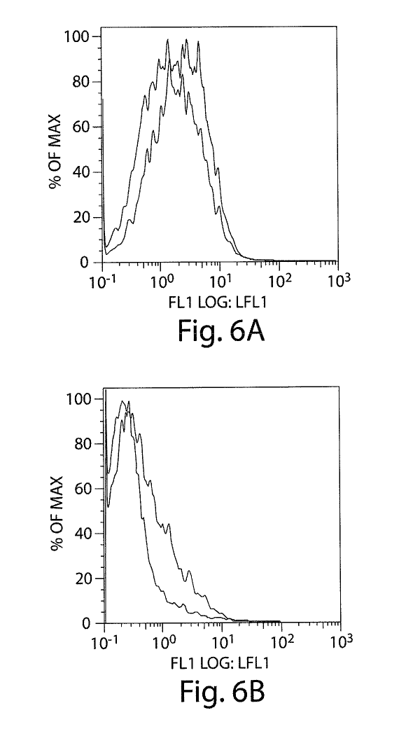

The data is shown in FIGS. 4-8. In the Histogram analyses of FIGS. 4-6 the Y axis represents cell number of the 5000 live cells versus the X axis which is a reflection of relative Fitc fluorescence versus Streptavidin-PE (eBioscience, Cat. #12-4317) that will bind with high affinity to cell-bound biotinylated peptides. The distance between the histogram from the isotype control staining versus the histogram reflecting the specific stain and is a measure of level of cell surface CLIP or the biotinylated peptide when stained with Streptavidin on a population of live Raji or Daudi cells as indicated.

At four hours, on both cell lines, significant evidence was observed that the biotinylated synthetic peptides bind with high affinity to the human B cell lines, Raji and Daudi, at 4 hours and less binding is observed at 24 hours. The cells were counter-stained with Fitc-Anti-CLIP antibodies and it was determined that treatment of cells with biotinylated peptides resulted in small decreases in cell surface bound CLIP at 4 hours and significant decreases at 24 hours when the competing peptides were FRIMAVLAS and TNP1. Thus the sequence of a bio-active peptide that has a high affinity for the majority of the HLA-DR, DP, and DQ alleles was predicted.

### Example 14

### Preclinical Work Using TNP Extract

A targeted peptide therapy (TNP extract) has been tested in humans internationally with documented success in lowering viral load, improving quality of life, and reducing quantifiable symptoms [Noveljic, Z., et al., Virological responses of treatment-naive stage CDC-2 HIV-1 positive subjects receiving VGV-1 injections in a blinded, placebo-controlled, multi-centre clinical trial. Retrovirology, 2006. 3: p. 73.]. The thymus derived peptides are good targets for Treg activation. There is evidence that Tregs usually have higher affinity for self and are selected in the thymus. Because TNP extracts (and thus thymus derived peptides) are derived from the thymus, the epitopes in the TNP extracts could be involved in Treg selection. When considered with the observations that there are aberrantly activated B cells that have switched to expression of non-thymically presented self peptides associated with MHC class II molecules, until purposely replaced, the B cell would not therefore be recognized by the Tregs until thymus derived peptides, or other appropriate self peptides, competitively replace the endogenous peptide in the groove of B cell MHC class II. The thymus derived peptides are likely enriched for the pool that selects Tregs in the thymus and these peptides are processed and presented in B cells differentially depending on disease state. Therefore, the partial success in reducing the HIV viral load that was observed in patients treated with the VGV-1 targeted peptide treatment (Described in detail below) is explained by the following series of observations: 1) gp120 from HIV polyclonally activates B cells that present conserved self antigens via MHC class II (or potentially MHC class I) and the activated B cells stimulate gamma delta T cells, 2) the VGV-1 targeted peptides bind with stronger affinity to the MHC molecules of the polyclonally activated B cell, 3) the consequence is activation and expansion of Tregs whose activation and expansion corresponds with decreased viral load, diminished γδT cell activation, and improvement as a result of inhibition of activation-induced cell death of non-Treg (referred to as conventional) CD4+ T cells.

Our model suggests that the success of this treatment involves binding of targeted to peptides from the TNP extract to cell surface Major Histocompatibility Complex (MHC) molecules on the activated B cell surface. MHC molecules are genetically unique to individuals and are co-dominantly inherited from each parent. MHC molecules serve to display newly encountered antigens to antigen-specific T cells. According to our model, if the MHC molecules bind a targeted peptide with greater affinity than the peptide occupying the groove of the MHC molecules on the activated B cell surface, the consequence will be activation of Treg cells that can dampen an inflammatory response. Tregs usually have higher affinity for self and are selected in the thymus [Wong, J., et al., Adaptation of TCR repertoires to self-peptides in regulatory and nonregulatory CD4+ T cells. J Immunol, 2007. 178(11): p. 7032-41.]. Therefore, because TNP extracts are derived from the thymus, it is reasonable to suggest that these epitopes could be involved in Treg selection. So then it follows that aberrantly activated B cells have switched to expression of non-thymically presented peptides. The thymus derived peptides of the invention may be represented in the pool that selects Tregs in the thymus. Loading of the thymic derived peptides onto activated B cells then provides a unique B cell/antigen presenting cell to activate the Treg. Thus, the thymus derived peptides can be used to re-direct the pathological innate immune response and activate important immunosuppressive T regulatory cells to reduce viral load and to diminish the loss of conventional, uninfected CD4+ T cells in HIV infection. The following preclinical studies were carried out in support of the model described herein.

Summary of Data from Nonclinical Studies

Pharmacology: The biological activity of the TNP extract can be demonstrated “in vitro” by its ability to precipitate two blood serum proteins and to bind with gp41, the HIV-1 envelope protein.

Serum Protein Binding: The in vitro reaction of TNP with serum proteins was observed by two-dimensional agarose electrophoresis. The method produces two protein precipitation lines when serum from an HIV negative individual is used. On the electrophoresis plate, these two precipitation bands are connected outside of the serum trace path and in front of the beta-1 microglobulin and alpha-2 macroglobulins. Preliminary data suggests that when serum from an HIV-1 infected individual is used, the precipitation bands are more distant from the serum trace. This may imply that the molecular weight of the precipitation band from an HIV positive person, is smaller than that of an HIV negative person.

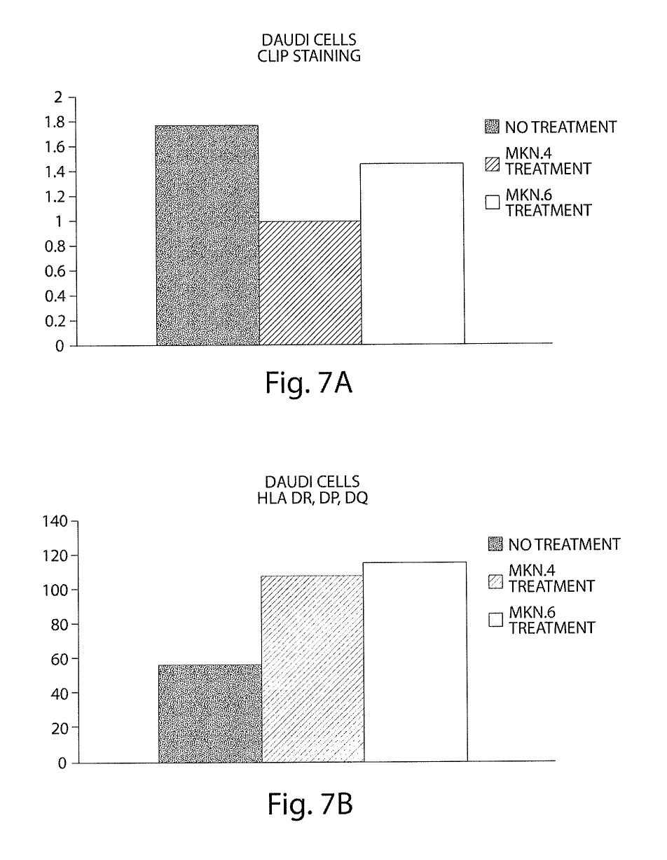

HIV-1 Envelope Protein (gp41) Binding Assay: TNP extract can interact with the gp41 HIV-1 envelope protein in vitro. Native gel electrophoresis indicated that the net charge of gp41 and TNP are different and opposite in polarity since they migrate in opposite directions (see FIG. 9). However, when mixed prior to electrophoresis, a single protein band was observed. The quantitative analysis indicates a binding stoichiometry of ˜1:0.8 for TNP to gp41. Lanes 1 and 2 were loaded with 7 μL TNP extract plus 6 μL gp41; lanes 3 and 4 with 7 μL TNP extract alone; and lane 5 was loaded with 6 μL gp41. Symbols indicate the position of the anode (−) and cathode (+). The gp41 fragment alone migrated toward the anode, but when mixed with TNP extract, the complex migrated toward the cathode.

Surface Plasmon Resonance (BIACORE) Binding Assay: A receptor assay has been developed and validated to characterize the quality of different batches of TNP preparations received from Viral Genetics Inc., CA. This assay is based on recently developed surface plasmon resonance (SPR) technology. Since biological activity always depends on a first binding step, the primary criterion for assessing biological activity is the ability of a compound to bind specifically to a ligand. The most sensitive biosensor instrument for detecting a compound-ligand interaction is the BIACORE. It directly captures any proteins including molecules from cytosolic or tissue extracts on sensor surface and measure binding kinetics with considerable ease and precision. It is widely used as the most sensitive method for measuring the active concentration of biomolecules and for their quality control.

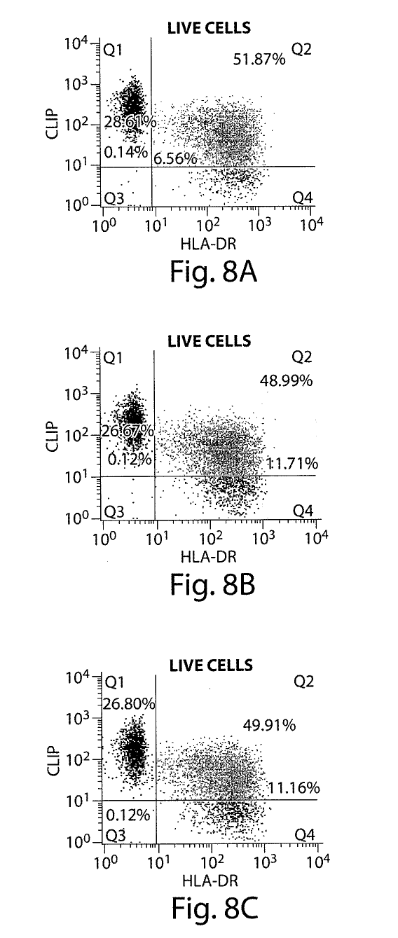

SPR is an optical technique that measures the refractive index change occurring at the sensor-fluid interface layer. To form microreaction chambers, a plastic plate is pressed into contact with a gold-coated glass chip (CM5), the surface of which is coated with an uncross-linked carboxymethylated dextran polymer matrix. The gold surface also serves as a partial mirror and optical port. Experiments are performed by a computer-program-driven robotics system to facilitate consistency. The injection of analyte across a ligand immobilized in the matrix produces a real-time change in refractive index signifying an increase in associated molecular mass. In our work TNP extract was the analyte used in the study, which binds to immobilized CD4 molecules. The data trace was a sensogram plotting response units (RU) against time (FIG. 10).

Initially buffer flows over the sensor surface coated with human CD4 molecules and a baseline level was established. Analyte (TNP extract) was injected into to microreaction chamber. During this injection the signal was related to complex formation (binding of TNP to CD4 molecules). After injection bound analyte (TNP) dissociated in buffer flow. A regeneration solution was injected to dissociate remaining analyte (TNP) from CD4 coated chamber.

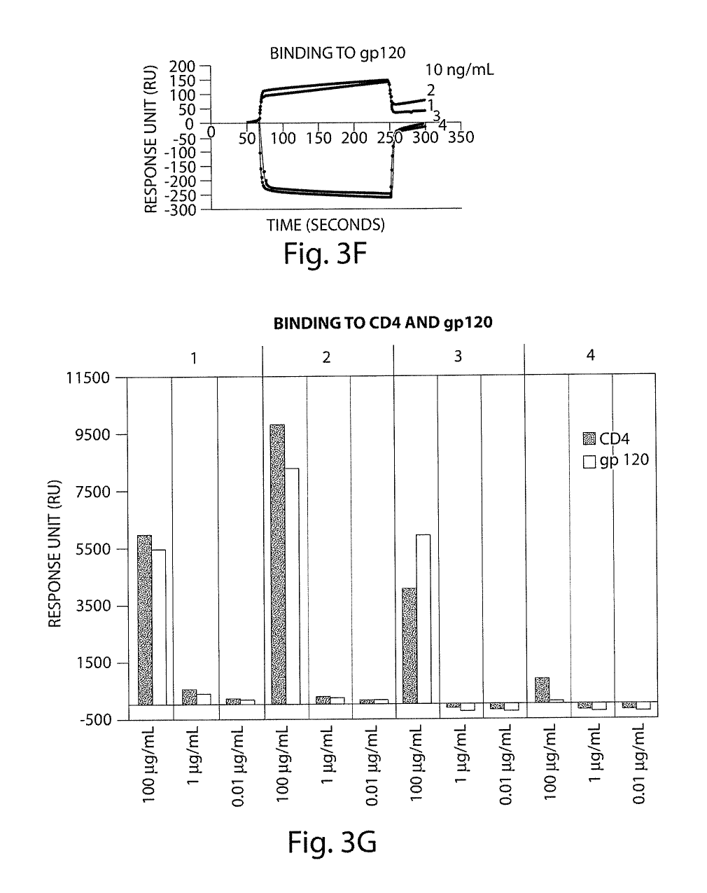

In our assay we measured the direct binding of an active component of TNP extract (containing unidentified biomolecule/s) to immobilized human CD4 molecules, which are binding receptors for HIV Immobilization of CD4 on the CM5 sensor surface was performed by standard amine coupling. The BIAcore system was equilibrated in running buffer (PBS, 0.05% Tween 20, 1 mM EDTA, pH 8.4) at a flow rate of 5 mL/min. The carboxymethylated dextran matrix was activated by injection of 35 mL of a solution containing NHS (0.05 M)/EDC (0.2 M) (50/50). Thirty-five microliters of CD4 at 500 mg/mL in citrate buffer (pH 4; 0.01 M) was injected. The deactivation of the remaining NHS-ester groups was performed by injection of 35 mL of ethanolamine hydrochloride (pH 8.5; 1 M). A regeneration of the sensor surface was done by the injection of 5 mL of 1M NaCl and 0.1 M NaOH. The experiments were performed at a constant flow rate of 5 mL/min of running buffer. All the reagents were prepared by dilution in running buffer. FIG. 5.3 shows kinetics of the specific binding and subsequent dissociation of TNP's active component with immobilized CD4. Maximal sample response (at the arrow) will be used in calculations and comparisons.

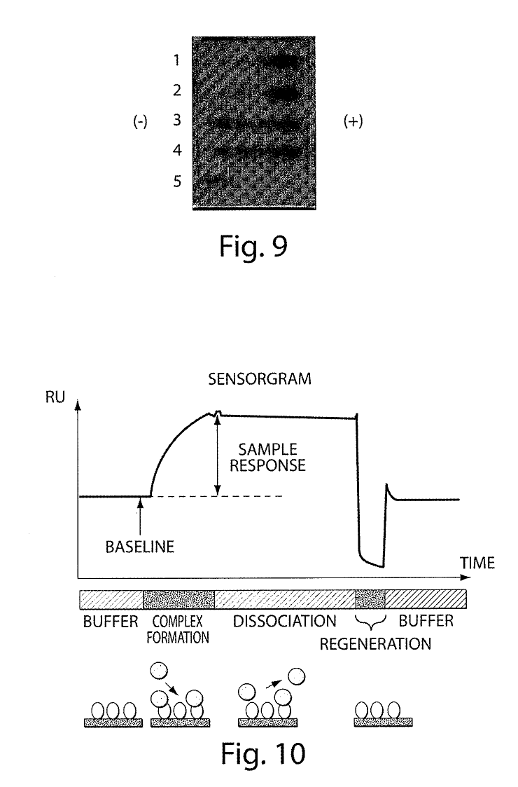

The data is shown in FIG. 11. The Biacore sensorgrams showing kinetics of the specific binding and subsequent dissociation of TNP's active component with immobilized CD4. Three sensorgrams correspond to different dilutions of one TNP sample received from Viral Genetics, Inc. The Arrow indicates the end of TNP injection and response at this time used for estimation of the sample's active component binding capacity at total concentration of sample. Such assays could be used to estimate active components within the different batches of TNP and compare one batch to another by binding activity to CD4.

The binding of thymus derived peptides as described herein can be validated by the independent assays described above.

Toxicology: In the early stages of development of TNP extract, a series of nonclinical studies were conducted to explore the toxicity profile of TNP administration, including acute toxicity testing in mice and rats, as well as a pilot 8-week toxicity study in rat. Pilot eight-week, repeat dose toxicity studies were conducted in mouse and rabbit to (Table 2). In addition to clinical observations, these studies also included histopathology assessment. TNP was well tolerated and only mild inflammatory response was observed in the lymph nodes of the animals. Each study is described in more detail below.

TABLE 2
Summary of Toxicity Studies

| | Species/ | Dose | | |
|---|---|---|---|---|
| Study Title | Strain | (# of animals & sex) | Duration | Findings |
| Toxicity Determination | Mouse/Non- | 0.8 mg (0.2 mL) | Twice | No mortality, |
| Study in Mice | Swiss | 5M | weekly | acceptable weight |
| (NamSA #95C 04723 | Albino CF1 | | for 8 | gain, no clinical |
| 00) | | | weeks | observations |
| Toxicity Determination | Rabbit/New | untreated (2M), | Twice | No mortality, |
| Study in Rabbit | Zealand | adjuvant only (2M), | weekly | acceptable weight |
| (CVD # X6000581) | | 2.8 mg (0.7 mL) TNP | for 8 | gain, no clinical |
| | | (5M) | weeks | observations |

Toxicity Determination Study in Mice (NAmSA, Study no. 95C 04723 00): The objective of this pilot study was to determine the general tolerance of the test article, VGV-1 (0.8 mg or 0.2 mL of 4 mg/mL microsuspension), following twice weekly intraperitoneal administration to the male, non-Swiss Albino CF1 derived mouse strain (n=5) for 8 consecutive weeks. When scaled to body surface area, 0.8 mg corresponds to a human equivalent dose of ˜3 mg/kg (assumptions: mouse body surface area of 0.007 m², and body surface area of 1.62 m² for a 60 kg human).

The drug product, VGV-1, is formulated as a sterile liquid microsuspension for intramuscular injection. Each single-use 2 mL vial of VGV-1 contains a formulation similar to the following and is adjusted based on dose:

| | | |
|---|---|---|
| Thymus Nuclear Protein (TNP) | 16 mg | Drug Substance |
| Sodium Chloride, USP/NF | 9 mg | Tonicity Agent |
| Sodium Acetate, Anhydrous, USP/NF | 6.8 mg | Buffering Agent |
| Aluminum Phosphate, USP | 2.26 mg | Suspending Agent |
| Sterile Water for Injection, USP | QS | |

Five (5), healthy, treatment naive post-weanling mice were intraperitoneally injected with VGV-1 at a dose of 0.2 mL. Animals were dosed twice per week for an eight-week period. Mice were observed for adverse reactions immediately after dosing. Body weights were recorded and gross pathology conducted on all animals at necropsy. Liver, kidney spleen and thymus were microscopically examined.

No mortality was observed during the study, and the range of body weight change during the study was acceptable. All animals appeared normal throughout the study. One of five animals escaped from its cage on Day 7. Therefore, only 4 animals were subsequently evaluated. Histopathology revealed minor, incidental changes to the thymic cortex and inflammation around the right kidney attributed to the intraperitoneal injection. Livers of two mice were characterized by coarse cytoplasmic clumping with vacuolation, slightly more prominent in the portal regions. The significance of this change is not known. Regional lymph nodes contained cortical follicles with active germinal centers and/or medullary plasmacytosis, indicating the lymph nodes were responding to an antigenic stimulus.

In summary, twice weekly intraperitoneal administration of VGV-1 for 8 weeks in mice was well-tolerated.

Toxicity Determination Study in Rabbit (CVD # X6000581): The objective of this pilot study was to determine the general tolerance of the test article, VGV-1 (2.8 mg or 0.7 mL of 4 mg/mL), following twice weekly intramuscular administration to male, New Zealand rabbits (n=5) for 8 consecutive weeks. Tolerance to vehicle (adjuvant only) was assessed in n=2 animals and n=2 animals were untreated controls. When scaled to body surface area, 2.8 mg corresponds to a human equivalent dose of ˜0.5 mg/kg (assumptions: rabbit body surface area of 0.15 m², and body surface area of 1.62 m² for a 60 kg human).

Five (5) rabbits were administered 0.7 mL of VGV-1 by intramuscular injection. Two rabbits were injected with 0.7 mL of vehicle (adjuvant only) and two rabbits were not injected and maintained as non-injected controls. Animals were dosed twice per week for an eight-week period. Rabbits were observed for adverse reactions immediately after dosing. Rectal temperature and body weights were recorded. Gross pathology conducted on all animals at necropsy, and the liver, kidney spleen, thymus, lymph nodes and brain were examined microscopically.

No mortality was observed during the study, body weight change during the study was acceptable, and all animals appeared normal. Minimal to mild lymphoid follicular hyperplasia was observed in the lymph nodes and considered to be a normal finding. No evidence of inflammation was present. Livers of both control and treated animals had minimal to mild peribiliary inflammation. Inflammation was lymphocytic, plasmacytic and sometimes heterophilic or a combination of cell infiltrates. The inflammation is likely associated with sub-clinical coccidial or bacterial infection. Minimal to mild renal lymphocytic and plasmactyic interstitial inflammation was observed in treated and one control animal. It is unclear what the mechanism is for this to finding, but it is considered incidental.

In summary, twice weekly intramuscular administration of VGV-1 for 8 weeks was well-tolerated in rabbits.

### Example 15

### Human Clinical Studies Using TNP Extracts

Viral Genetics Inc. has conducted a total of six ex-USA human clinical trials of VGV-1. All trials were conducted on HIV positive individuals. In all 6 studies, subjects received 8 mg VGV-1 as an intramuscular injection of 2.0 mL of a 4.0 mg/mL suspension of TNP, twice a week for 8 weeks for a total of 16 doses. A summary of the clinical studies is presented in Table 3. The results of the studies described in this Example and Example 16 have been previously published or made publicly available. The studies are disclosed herein provide a study of the human clinical trials performed with the TNP extract, from which the instant thymus derived peptides are derived.

TABLE 3
Ex-US Clinical Studies of VGV-1

| | | | | | Post- | |
|---|---|---|---|---|---|---|
| | Year | Number of | CDC | | Treatment | Trial |
| Study | Initiated | Subjects | Stage | Study Comments | Follow-up | Status |
| Infectious Disease | 1995 | 4 | Varied | Investigator | Varied, up to | Complete |
| Hospital | | | | Study, Open Label | 18 months | |
| University of | | | | | | |
| Sofia | | | | | | |
| Sofia, Bulgaria | | | | | | |
| Private Clinic | 1996 | 15 | Varied | Open-label | 18 months | Complete |
| Tijuana, Mexico | | | | | | |
| Infectious Disease | 1997 | 20 VGV-1 | Varied | Single-masked | 9 months | Complete |
| Hospital | | 10 protease | | | | |
| University of | | inhibitor only | | | | |
| Sofia | | | | | | |
| Sofia, Bulgaria | | | | | | |
| IMSS Hospital 25 | 1999 | 10 | 3 | ART resistant | 6 months | Complete |
| Monterrey, | | | | | | |
| Mexico | | | | | | |
| Ditan Hospital | 2003 | 34 | 3 | Treatment-naive | 9 months | Complete |
| Beijing China | | | | | | |
| Multi-site | 2004 | 137 | 2 | Randomized, | 6 months | On-going |
| South Africa | | | | double-blind, | | |
| | | | | placebo controlled | | |

1. Pilot Study: Infectious Disease Hospital, University of Sophia (Sophia, Bulgaria)

Objective: The objective of this exploratory study was to investigate the initial safety of VGV-1.

Study Design: This was an Investigator-sponsored study initiated in 1995 by Dr. Kostadin Kostov to explore the effect of VGV-1 in HIV-1 infected patients. Four subjects were administered 2 mL of VGV-1 at a concentration of 4 mg/mL by to intramuscular injection, twice a week for a total of 8 weeks from April to May 1995. The patients continued to receive concomitant HIV medication.

TABLE 4
Listing of HIV-Related Concomitant Medication

| Patient | | | | Stop | Duration |
|---|---|---|---|---|---|
| Number | Visit Date | Medication | Start Date | Date | in Days |
| 1 | 01APR1995 | AZT | 01APR1994 | — | Continuous |
| 4 | 01APR1995 | 3TC | 01APR1994 | — | Continuous |
| 4 | 01APR1995 | AZT | 01APR1994 | — | Continuous |

Total number of patients: 4
Number of patients on HIV-related Medication: 2 (50%)

Safety Analysis: In the pilot study there were a total of 24 adverse events reported in 4 patients during the treatment period. None of the patients were assessed during the follow-up period, hence no adverse event data was captured. The distribution of the number of adverse events by body system and preferred term is shown in Table 5. Seven adverse events were digestive system related, 6 were urogenital-system related and 11 were body-as-a-whole related. There were no serious adverse events or deaths in the study.

TABLE 5
Number and Incidence of Adverse Events by Body System and
Treatment Group (Pilot Bulgaria Study)

| | Treatment Period |
|---|---|
| | (n = 4) |
| NUMBER OF EVENTS | 24 |
| BODY AS A WHOLE | 11 (47%) |
| Fatigue | 8 (72%) |
| Flank Pain | 3 (27%) |
| DIGESTIVE SYSTEM | 7 (28%) |
| Dry Mouth | 6 (86%) |
| Taste Perversion | 1 (14%) |
| UROGENITAL SYSTEM | 6 (25%) |
| Disuria | 6 (100%) |

2. Pilot Study: Mexico Clinical Trial (Tijuana, Mexico)

Objective: The objective of this pilot study was to investigate the safety of VGV-1 and its ability to inhibit or stop HIV-1 viral replication.

Study Design This first Mexican human clinical trial consisted of fifteen HIV infected patients, administered 8 mg VGV-1 (2 mL of 4 mg/mL TNP), twice a week for a total of 8 weeks. Fourteen patients began treatment in March through May 1996, and completed treatment from April through July 1996, with one patient beginning November 1995 and completing in January 1996. The study was open-label; however, to the independent laboratory generating the quantitative results was masked to the patient treatment codes.

Fourteen patients completed the full treatment course with follow-up ranging up to 331 days from the final injection. Throughout the study, patients demonstrated no significant deleterious effects from VGV-1 treatment as measured by clinical examination, subjective patient questioning, routine blood chemistries or by immunologic or virologic makers. The patients continued to receive concomitant HIV medication.

Safety Analysis: Fifteen patients with various CDC stage were enrolled in the study. There were a total of 76 adverse events (66 in treatment and 10 in post-treatment period). Thirty adverse events were digestive system related, 24 were nervous system related and 22 were body-as-a-whole related. There were no Grade IV (Very Severe) adverse events in the study and most of the adverse events were Grade I (Mild) severity, some adverse events were Grade II and III during the treatment period but none during the follow-up period. Thirty adverse events (40%) were digestive system related, 24 (32%) were nervous-system related and 22 (29%) were body-as-a-whole related. There were no serious adverse events or deaths reported. Overall, based on the available data, the study showed VGV-1 treatment was well tolerated in the treated patients.

Activity and Safety Markers: Viral load was measured by the Roche PCR assay and CD4 cell counts were performed during the trial.

TABLE 6
Baseline CD4 and HIV/RNA (log 10)
Fifteen patients were enrolled in the study with various CDC stage. The
mean CD4 at the baseline was 227.8 (stdev 193.6) ranged from 7 to 534.
The mean LOG RNA at the baseline was 4.74LOG (stdev 0.95LOG)
ranged from 1.85LOG to 5.83LOG.

| Variable | N | Median | Mean | Std Dev | Minimum | Maximum |
|---|---|---|---|---|---|---|
| CD4_BS | 15 | 182.50 | 227.80 | 193.60 | 7.00 | 534.00 |
| RNA_BS | 15 | 4.88 | 4.74 | 0.95 | 1.85 | 5.83 |

TABLE 7
Changes in CD4 Cell Count from Baseline at Different Time Points
(Months)
With the intent-to-treat population the study showed that mean CD4
change from baseline was increased at month 3 (by 40), but decreased
at month 4 and month 5 (by 70) and at month 9 (by 42).

| Variable | N | Median | Mean | Std Dev | Minimum | Maximum |
|---|---|---|---|---|---|---|
| CD4_BS | 15 | 182.5 | 227.8 | 193.6 | 7 | 534 |
| chcd4_m3 | 9 | −5.0 | 40.0 | 139.4 | −104 | 258.5 |
| chcd4_m4 | 5 | −92.5 | −71.4 | 62.5 | −152 | −5.5 |
| chcd4_m5 | 8 | −10.5 | −70.4 | 166.9 | −468 | 38.5 |
| chcd4_m6 | 1 | 34.0 | 34.0 | | 34.0 | 34.0 |
| chcd4_m9 | 9 | −68.5 | −42.0 | 129.5 | −184.5 | 167.5 |

TABLE 8
Change in Plasma HIV/RNA (log 10) from Baseline at Different Time
Points (Months)
With the intent-to-treat population the study showed that mean LOG
RNA change from baseline was reduced at month 3 and month 4
(by about 0.5LOG and 0.32LOG, respectively), but returned to baseline
level at month 5 then reduced at month 9 (by about 0.47LOG).

| Variable | N | Median | Mean | Std Dev | Minimum | Maximum |
|---|---|---|---|---|---|---|
| RNA_BS | 15 | 4.88 | 4.74 | 0.95 | 1.86 | 5.84 |
| chrna_m3 | 9 | −0.77 | −0.48 | 0.69 | −1.35 | 0.72 |
| chrna_m4 | 5 | −0.21 | −0.32 | 0.27 | −0.69 | −0.03 |
| chrna_m5 | 8 | −0.09 | 0.13 | 1.02 | −1.34 | 2.3 |
| chrna_m6 | 1 | −1.29 | −1.29 | | −1.29 | −1.29 |
| chrna_m9 | 9 | −0.06 | −0.47 | 1.31 | −2.39 | 1.61 |

Summary: These data suggested that VGV-1 treatment in HIV-1 infected patients was safe and well tolerated in this human trial. There was a decrease in CD4 cells observed in this trial whose trajectory may be consistent with the natural progression of disease. However, the observed changes in HIV-1 RNA were not necessarily consistent with the expected increases observed in the natural history of HIV-1 infection.

3. Infectious Disease Hospital, University of Sophia Study (Sofia, Bulgaria)

Objective: The objective of the study was to demonstrate the clinical safety and antiviral efficacy of VGV-1 in treating HIV-1 infected human subjects

Study Design: Thirty (30) HIV-1 and AIDS patients were enrolled on the study. 20 subjects were randomized to the VGV-1 treatment and 10 subjects were randomized to the protease inhibitor treatment. The 20 subjects randomized to the VGV-1 treatment were further subdivided into 3 subgroups (Table 9). The patients continued to receive concomitant HIV medication.

TABLE 9
Stratification of Subjects by CD4 Count

| | | |
|---|---|---|
| Group1: | CD4 < 200 cells in cmm | n = 4 |
| Group 2: | CD4 = 200-500 cells in cmm | n = 6 |
| Group 3: | CD4 > 500 cells in cmm | n = 10 |

The VGV-1 dose was 8 mg (2 mL intramuscular injection containing 4 mg/mL of TNP). The subjects received two injections per week, on two consecutive days for a period of eight weeks for a total of sixteen intramuscular injections. Patients began receiving treatment with VGV-1 in June 1997 and completed in July and August 1997.

The subjects were followed up for ten months post treatment. Both baseline treatment and post treatment examinations were conducted. Clinical laboratory data was collected throughout the study and mean data.

Safety Analysis: The safety data is presented for the 20 patients receiving VGV-1 during the 8-week treatment period and for the 10-month post-treatment follow-up period.

There were a total of 312 adverse events (300 in treatment and 12 in the post-treatment period). Ninety-one adverse events were nervous system related, 90 were digestive system related, 13 were urogenital system related, 6 were rashes skin-related, 103 were body-as-a-whole related, 2 were respiratory-related, and 13 for hemic lymphatic system related. Most of the adverse events occurred during the treatment period but six patients also experienced some adverse events in the follow-up period. There were no deaths or serious adverse events reported during the study. There were no clinically significant changes in mean values for routine laboratory parameters during the course of the study.

Overall, based on the available data, the study showed the VGV-1 treatment was well tolerated in the treated patients.

Activity and Safety Markers: Viral load was measured by the Roche PCR assay and CD4 cell counts were performed during the trial

TABLE 10
Baseline CD4 and HIV/RNA (log 10)
Twenty patients were enrolled in the study with various CDC stage.
The mean CD4 at the baseline was 432 (stdev 252) ranged from
94.5 to 856. The mean LOG RNA at the baseline was 4.22LOG
(stdev 0.75LOG) ranged from 3.11LOG to 5.68LOG.

| Variable | N | Median | Mean | Std Dev | Minimum | Maximum |
|---|---|---|---|---|---|---|
| CD4_BS | 20 | 309.5 | 431.90 | 252.43 | 94.50 | 856.00 |
| RNA_BS | 20 | 4.18 | 4.23 | 0.75 | 3.11 | 5.68 |

TABLE 11
Changes in CD4 Cell Counts from Baseline at Different Time Points
(Months)
With the intent-to-treat population the study showed that mean CD4
change from baseline was increased at all follow-up months, month 3
(by 18), month 4 (by 27.5) and month 5 (by 6) and at month 9 (by 26).

| Variable | n | Median | Mean | Std Dev | Minimum | Maximum |
|---|---|---|---|---|---|---|
| CD4_BS | 20 | 309.50 | 431.90 | 252.43 | 94.50 | 856.00 |
| chcd4_m3 | 20 | −17.00 | 18.00 | 114.96 | −137.00 | 379.50 |
| chcd4_m4 | 18 | 20.00 | 27.53 | 147.06 | −127.00 | 561.50 |
| chcd4_m5 | 18 | −11.75 | 6.33 | 108.72 | −149.00 | 205.50 |
| chcd4_m6 | 20 | 12.25 | 25.90 | 188.51 | −243.00 | 655.50 |
| chcd4_m9 | 0 | | | | | |

TABLE 12
Changes in Viral Load from Baseline HIV/RNA (log 10) at Different
Time Points (Months)
With the intent-to-treat population the study showed that mean LOG
RNA change from baseline remained unchanged, at month 3 (by 0.06),
month 6 (by 0.01), but reduced by 1 LOG at month 9.

| Variable | N | Median | Mean | Std Dev | Minimum | Maximum |
|---|---|---|---|---|---|---|
| RNA_BS | 20 | 4.18 | 4.23 | 0.75 | 3.11 | 5.68 |
| chrna_m3 | 17 | −0.07 | −0.06 | 0.57 | −1.42 | 0.80 |
| chrna_m4 | 0 | | | | | |
| chrna_m5 | 0 | | | | | |
| chrna_m6 | 19 | 0.09 | −0.01 | 0.79 | −1.71 | 1.19 |
| chrna_m9 | 19 | −0.91 | −1.00 | 0.62 | −2.16 | 0.09 |

Summary: These preliminary findings suggested that VGV-1 treatment in HIV-1 infected patients was safe and well tolerated. A small increase in CD4 cell counts was observed during this trial. In addition a decrease in HIV RNA was observed during this trial.

4. IMSS Hospital 25 Study (Monterrey, Mexico)

The results of the following study were published in HIV AIDS Rev, 2004, 3(3): 8-13).

Objective: The study objective was to demonstrate the clinical safety and antiviral efficacy of VGV-1 in treating ten HIV-1infected, AIDS patients who have developed resistance to Multi-Antiviral Drug Cocktail Therapies.

Study Design: Ten (10) HIV-infected AIDS and HIV patients who have developed clinical resistance to multi drug cocktail therapies were given intramuscular injections of 8 mg VGV-1 (2 mL of 4 mg/mL suspension of TNP). The subjects received two injections per week, on two consecutive days for a period of eight weeks for a total of sixteen VGV-1 intra muscular injections. Patients received VGV-1 treatment from September to November 1999. The patients continued to receive concomitant HIV medication. Clinical laboratory data was collected throughout the study.

Safety Analysis: Ten patients with the CDC stage III were enrolled in the study for an 8-week treatment period and 6-month post-treatment follow-up period. There were a total of 250 adverse events, all during the treatment. Nine adverse events were urogenital-system related, 102 adverse events were digestive system related, 41 were nervous system related, 2 were respiratory system related, 21 rashes skin-related and 66 were body-as-a-whole related. There were no Grade IV (Very Severe) adverse events in the study and most of the adverse events were Grade I (Mild) severity, some adverse events were Grade II and III during the treatment period but none during the follow-up period. The adverse events with all severities showed that 8 (3.6%) adverse events were urogenital-system related, 102 (41%) were digestive-system related, 41 (16%) were nervous-system related, 21 (8%) rashes skin-related, 12 (4.4%) were respiratory related and 66 (26%) were body-as-a-whole related. There were no deaths or serious adverse events reported during the study. There were no clinically significant changes in mean values for routine laboratory parameters during the course of the study.

Overall the study showed VGV-1 treatment was well tolerated in the treated patients.

Activity and Safety Markers: Viral load was measured by the Roche PCR assay and CD4 cell counts were performed during the trial (Tables 13 to 15). The activity data collected in this study was analyzed by individual subject and the results of that analysis published in HIV AIDS Rev, 2004; 3(3): 8-13.

TABLE 13
Baseline CD4 and HIV/RNA (log 10)
Ten patients were enrolled in the study with CDC stage III. The mean
CD4 at the baseline was 135.6 (stdev 80) ranged from 30 to 304.
The mean LOG RNA at the baseline was 3.87LOG (stdev 0.44LOG)
ranged from 3.32LOG to 4.6LOG.

| Variable | N | Median | Mean | Std Dev | Minimum | Maximum |
|---|---|---|---|---|---|---|
| CD4_BS | 10 | 139.0 | 135.6 | 79.0 | 30.0 | 304.0 |
| RNA_BS | 10 | 3.91 | 3.87 | 0.44 | 3.32 | 4.60 |

TABLE 14
Changes in CD4 Cell Count from Baseline at Different Time Points
(Months)
With the intent-to-treat population the study showed that mean CD4
change from baseline was increased at month 3 (by 12) but decreased
at month 6 (by 18.5).

| Variable | N | Median | Mean | Std Dev | Minimum | Maximum |
|---|---|---|---|---|---|---|
| CD4_BS | 10 | 139.0 | 135.6 | 78.995 | 30 | 304 |
| chcd4_m3 | 10 | 17.5 | 12.0 | 30.57 | −38.00 | 61.0 |
| chcd4_m4 | 0 | | | | | |
| chcd4_m5 | 0 | | | | | |
| chcd4_m6 | 10 | −3.00 | −18.50 | 29.878 | −66.0 | 13.0 |
| chcd4_m9 | 0 | | | | | |

TABLE 15
Changes in Viral Load HIV/RNA (log 10) from Baseline at Different
Time Points (Months)
With the intent-to-treat population the study showed that mean Log
RNA change from baseline had 1 Log reduction at both month 3
and month 6.

| Variable | N | Median | Mean | Std Dev | Minimum | Maximum |
|---|---|---|---|---|---|---|
| RNA_BS | 10 | 3.91 | 3.87 | 0.44 | 3.32 | 4.60 |
| chrna_m3 | 10 | −0.91 | −0.98 | 1.21 | −2.68 | 0.46 |
| chrna_m4 | 0 | | | | | |
| chrna_m5 | 0 | | | | | |
| chrna_m6 | 10 | −1.08 | −1.08 | 1.10 | −2.68 | 0.32 |
| chrna_m9 | 0 | | | | | |

Summary: These preliminary findings suggested that VGV-1 treatment in HIV-1 infected patients was safe and well tolerated in patients on concomitant antiviral drugs. A small decrease in CD4 cell counts was observed during this trial. In addition a decrease in HIV RNA was observed during this trial.

5. Assessment of Safety and Efficacy of Thymus Nuclear Protein Injections for Treating HIV-1 Infected Patients (China AIDS Project)

Objective: The objective of this study was to assess the safety and efficacy of VGV-1 in treating HIV-1 infected patients in the late stages of AIDS.

Study Design Approximately 34 subjects with CD4+ counts of less than 200 were enrolled in this single blind study. Patients were not taking concomitant antiviral drugs during this trial. All subjects received 8 mg VGV-1 (2 mL intramuscular injections of 4 mg/mL TNP), twice a week on two consecutive days for a period of eight weeks. Patients received VGV-1 treatment from April to May 2003. Clinical laboratory data was collected throughout the study.

Safety Analysis: Thirty-four patients with the CDC stage III were enrolled in the study for the 8-week treatment period and 9-month post-treatment follow-up period. There were a total of 1406 adverse events (1222 in treatment and 184 in post-treatment period). Adverse events were elicited through specific review of systems rather than through spontaneous reporting and were collected at each visit regardless of whether present at baseline or continuing unchanged. This resulted in the extraordinarily high number of reported events. 580 adverse events were digestive system related, 222 were nervous system related, 3 respiratory-related and 584 were body-as-a-whole related. There were no Grade IV (Very Severe) adverse events in the study and most of the to adverse events were Grade I (Mild) severity, some adverse events were Grade II and III during the treatment period and the follow-up period. There were no clinically significant changes in mean values for routine laboratory parameters during the course of the study.

Deaths Reported (Post-Treatment) Three randomized patients expired during this study. The study ended in November 2003 and none of the Case Report Forms received by the Sponsor from this trial had indicated that any serious adverse events had occurred. However, in April 2004, the Sponsor received information in a letter from Ditan Hospital that three patients (Nos. 012/TZL, 021/WLH, and 027/ZRHV) had died during the post-treatment follow-up period. A summary of these 3 death reports is as follows:

Patient 012/TZL, a 52-year-old Asian female, was enrolled on 6 Mar. 2003, to receive open-label treatment with Thymus Nuclear Protein (TNP) suspension, 8 mg IM twice a week for a period of eight (8) weeks. The patient tested positive for HIV on November 2002, had the diagnosis of AIDS and had never been treated with antiviral agents. She had a medical history of herpes, shingles, chicken pox and skin rash. Concomitant medications included SMZco (trimethoprim and sulfamethoxazole) for PCP one qd (6 to 11 Mar. 2003), Vitamin C 200 mg bid (starting 6 Mar. 2003), and Vitamin B complex two bid (starting 6 Mar. 2003). Baseline (Day −14) physical exam was significant for skin rash and the laboratory data were significant only for an elevated LDH of 218 U/L; the CD4 count was 112/μL with a CD4/CD8 ratio of 0.09. The patient's symptoms on Day −14 included moderate fatigue and abdominal pain and mild anorexia and dry mouth. Baseline assessment on Day −7 was essentially unchanged; the CD4 count was 167/μL with a CD4/CD8 ratio of 0.09. The patient began treatment with study drug on 21 Mar. 2003 and received all 16 injections as scheduled. The treatment was tolerated well with only mild to moderate dry mouth as a persistent complaint and occasional mild headache, malaise, and insomnia. Follow-up laboratory assessments on 14 Apr. 2003 were unremarkable; the CD4 count was 120/μL with a CD4/CD8 ratio of 0.12. The patient completed treatment on 9 May 2003 and post-treatment laboratory assessments on 16 May 2003 (Day 60) showed increased total protein and serum globulins in addition to elevated LDH but were otherwise unremarkable; the CD4 count was 112/μL with a CD4/CD8 ratio of 0.16. The patient was last seen on 16 Aug. 2003 (Day 120) when she was found to have findings on physical exam related to the digestive system and she complained of mild diarrhea. There were no significant changes in laboratory findings; the CD4 count was 143/μL with a CD4/CD8 ratio of 0.22. According to a follow-up report from the investigator at Ditan Hospital the patient began experiencing fevers and severe skin rash after returning to her hometown. She did not receive any antiretroviral or prophylactic treatments. She subsequently developed shortness of breath which gradually progressed and she died on 9 Aug. 2003, three months after the last treatment, due to respiratory failure. The cause of death was believed to be PCP secondary to AIDS.

Patient 021/WLH, a 33-year-old Asian male, was enrolled on 6 Mar. 2003, to receive open-label treatment with Thymus Nuclear Protein (TNP) suspension, 8 mg IM twice a week for a period of eight (8) weeks. The patient tested positive for HIV on November 2001, had the diagnosis of AIDS and had never been treated with antiviral agents. He was believed to have been infected after a blood transfusion. He had a medical history of Hepatitis A, shingles, and thrush. Concomitant medications included SMZco (trimethoprim and sulfamethoxazole) two bid (6 Mar. 2003 to 23 Apr. 2003) increased to three bid (24 Apr. 2003), Vitamin C 200 mg bid (starting 6 Mar. 2003), Vitamin B complex two bid (starting 6 Mar. 2003), fluconazole 100 mg qd (starting 13 Mar. 2003), and ceftriaxone 2 g qd (18 to 23 Apr. 2003). Baseline (Day −14) physical exam was significant for enlarged lymph nodes and an abnormal respiratory exam. The laboratory data were significant only for a mildly elevated WBC count of 13.2×10⁹/L and increased serum globulins; the CD4 count was 12/μL with a CD4/CD8 ratio of 0.01. The patient's symptoms on Day −14 included mild fatigue, malaise, dry mouth, back/flank pain, headache, insomnia, and dizziness. Baseline assessment on Day −7 was essentially unchanged; the CD4 count was 15/μL with a CD4/CD8 ratio of 0.01. The patient began treatment with study drug on 21 Mar. 2003 and received all 16 injections as scheduled. The treatment was tolerated well with the baseline symptoms as persistent complaints at a mild to moderate level and occasional mild anorexia and diarrhea. Follow-up laboratory assessments on 14 Apr. 2003 were unremarkable except for elevated LDH at 284 U/L; the CD4 count was 10/μL with a CD4/CD8 ratio of 0.01. The patient completed treatment on 9 May 2003 and post-treatment laboratory assessments on 16 May 2003 (Day 60) showed no significant changes; the CD4 count was 7/μL with a CD4/CD8 ratio of 0.01. This was the patient's last visit. According to a follow-up report from the investigator at Ditan Hospital the patient began experiencing high fevers one week after returning to his hometown. He subsequently died (date unknown), due to severe infection and renal failure.

Patient 027/ZRHV, a 40-year-old Asian male, was enrolled on 6 Mar. 2003, to receive open-label treatment with Thymus Nuclear Protein (TNP) suspension, 8 mg IM twice a week for a period of eight (8) weeks. The patient tested positive for HIV in 2001, had the diagnosis of AIDS and had never been treated with antiviral agents. He had a medical history of tuberculosis. Concomitant medications included SMZco (trimethoprim and sulfamethoxazole) one qd (6 to 11 Mar. 2003), transiently increased to two qd (17 to 24 Apr. 2005), Vitamin C 200 mg bid (starting 6 Mar. 2003), Vitamin B complex two bid (starting 6 Mar. 2003), and Pen-G (11 to 17 Apr. 2003). Baseline (Day −14) physical exam was significant for skin rash and abnormal respiratory exam. The laboratory data were significant only for a slightly decreased WBC count of 3.47×10⁹/L and slightly elevated LDH of 241 U/L; the CD4 count was 100/μL with a CD4/CD8 ratio of 0.29. The patient's symptoms on Day −14 included very severe taste perversion, severe fatigue, malaise, and anorexia, and mild dry mouth back/flank pain. Baseline assessment on Day −7 was essentially unchanged; the CD4 count was 36/μL with a CD4/CD8 ratio of 0.13. The patient began treatment with study drug on 21 Mar. 2003 and received all 16 injections as scheduled. The treatment was tolerated well with the baseline symptoms as persistent complaints at a mild to moderate level and occasional mild to moderate dizziness and insomnia. Follow-up laboratory assessments on 14 Apr. 2003 were unremarkable; the CD4 count was 96/μL, with a CD4/CD8 ratio of 0.88. The patient completed treatment on 9 May 2003 and post-treatment laboratory assessments on 16 May 2003 (Day 60) showed increased total protein and serum globulins in addition to elevated LDH of 319 U/L but were otherwise unremarkable; the CD4 count was 28/μL, with a CD4/CD8 ratio of 0.18. The patient was last seen on 16 Aug. 2003 (Day 120) when there were no significant changes in physical exam or in laboratory findings and no new complaints; the CD4 count was 12/μL, with a CD4/CD8 ratio of 0.07. According to a follow-up report from the investigator at Ditan Hospital the patient began experiencing recurrent fevers after returning to his hometown. He did not receive any antiretroviral or prophylactic treatments. He subsequently died on 12 Oct. 2003, five months after the last treatment, due to unknown causes.

Activity and Safety Markers: Viral load was measured by the Roche PCR assay and CD4 cell counts were performed during the trial

TABLE 16
Baseline CD4 and HIV/RNA (log 10)
Thirty-four patients were enrolled in the study with CDC stage III.
The mean CD4 at the baseline was 94 (stdev 49) ranged from 11.5
to 195.5. The mean LOG RNA at the baseline was 4.9LOG
(stdev 0.61LOG) ranged from 3.16LOG to 5.79LOG.

| Variable | N | Median | Mean | Std Dev | Minimum | Maximum |
|---|---|---|---|---|---|---|
| CD4_BS | 34 | 100.25 | 93.65 | 48.84 | 11.50 | 195.50 |
| RNA_BS | 34 | 5.02 | 4.90 | 0.606 | 3.16 | 5.79 |

TABLE 17
Changes in CD4 Cell Count from Baseline at Different Time Points
(Months)
With the intent-to-treat population the study showed that mean CD4
change from baseline was virtually unchanged at all study months month
4 (by 2.6) and month 5 (by 6.4) and at month 9 (by 2.34).

| Variable | N | Median | Mean | Std Dev | Minimum | Maximum |
|---|---|---|---|---|---|---|
| CD4_BS | 34 | 100.25 | 93.65 | 48.84 | 11.50 | 195.50 |
| chcd4_m3 | 0 | | | | | |
| chcd4_m4 | 31 | −2.50 | −2.597 | 31.67 | −76.00 | 66.50 |
| chcd4_m5 | 28 | 2.25 | 6.429 | 49.25 | −99.00 | 100.50 |
| chcd4_m6 | 0 | | | | | |
| chcd4_m9 | 25 | −3.00 | −2.34 | 61.67 | −98.50 | 113.50 |

TABLE 18
Changes in Viral Load HIV/RNA (log 10) from Baseline at Different
Time Points (Months)
With the intent-to-treat population the study showed that mean LOG
RNA change from baseline was reduced at all follow-up months, month
4 (by 0.03LOG), month 5 (by 0.7LOG) and month 9 (by 0.43LOG).

| Variable | N | Median | Mean | Std Dev | Minimum | Maximum |
|---|---|---|---|---|---|---|
| RNA_BS | 34 | 5.02 | 4.90 | 0.61 | 3.16 | 5.79 |
| chrna_m3 | 0 | | | | | |
| chrna_m4 | 31 | 0.10 | −0.03 | 0.77 | −2.99 | 1.36 |
| chrna_m5 | 28 | −0.52 | −0.70 | 1.11 | −3.54 | 1.23 |
| chrna_m6 | 0 | | | | | |
| chrna_m9 | 24 | 0.01 | −0.43 | 1.46 | −4.04 | 1.73 |

Summary: These preliminary findings suggested that VGV-1 treatment in HIV-1 infected patients was safe and well tolerated. A small decrease in CD4 cell counts was observed during this trial. In addition a decrease in HIV RNA was observed during this trial.

### Example 16

### A Phase 3 Prospective, Masked, Randomized, Placebo Controlled Multi-Center Study to Assess the Safety and Efficacy of VGV-1 Injections for Treating Stage CDC-2 HIV Infected Subjects (South Africa)

Objective: The purpose of this study is to collect additional clinical data on the safety profile and the efficacy of VGV-1 given in 2.0 mL intra-muscular injections (4 mg/mL) of Sterile VGV-1 suspension or placebo suspension two times a week on two consecutive days for a period of eight (8) weeks in subjects who have been identified as having stage CDC-2 AIDS. Trial enrollment was completed in March, 2005 with 137 patients randomized

Study Design:
Phase 3 Prospective, masked, randomized, placebo controlled multi-center study.
Group I Study Arm: Subjects who have been identified as having stage CDC-2 HIV disease, received 2.0 mL intra-muscular injections (4 mg/mL) of Sterile VGV-1 suspension two times a week on two consecutive days for a period of eight (8) weeks.
Group II Control Arm: Subjects who have been identified as having stage CDC-2 HIV disease, received intra-muscular injections of 2.0 mL of Sterile Control Suspension two times a week on two consecutive days for a period of eight (8) weeks.
On day −14 and −7 (Baseline 1 and Baseline 2) prior to the start of the study (first injection of VGV-1 suspension and Sterile Control Suspension), the subjects completed physical examination, clinical examination, Body mass and weight measurements, blood chemistry, CD4, CD8, CD3 measurements as well as quantitative PCR and quantitative PBMC measurements were conducted.
The subjects had to meet the requirements of the Inclusion and Exclusion criteria
The subjects of the Group I study arm were injected intramuscularly with VGV-1 suspension on the following days: 1 and 2, days 8 and 9, days 15 and 16, days 22 and 23, days 29 and 30, days 36 and 37, days 43 and 44, days 50 and 51.
The subjects of the Group II control arm were injected intramuscularly with Control suspension on the following days: 1 and 2, days 8 and 9, days 15 and 16, days 22 and 23, days 29 and 30, days 36 and 37, days 43 and 44, days 50 and 51.
All subjects had follow-up visits during the VGV-1 and Control treatments on days 23 and 51, during which time complete physical examination, clinical examination, body mass and weight measurements, blood chemistry, CD4, CD8, CD3 measurements as well as quantitative PCR and quantitative PBMC measurements were conducted.
All subjects were scheduled with follow-up visits post the VGV-1 and Control treatments on days 90, day 120, day 150 and day 240 during which time complete physical examination, clinical examination, body mass and weight measurements, blood chemistry, CD4, CD8, CD3 measurements as well as quantitative PCR and quantitative PBMC measurements are conducted.

Safety Endpoints:

The safety end points were measured by means of Medical History, Physical Examination, Clinical Disease Progression, Routine Laboratory Tests and Adverse Events.
Absence of serious adverse complications and adverse experiences through 120 days post-treatment.
Absence of clinical data suggestive of renal or hepatic damage, or the disabling of the hemopoietic system through the analysis of Blood Chemistry results.

Activity and Safety Markers:
Viral load reduction of >1 log or non-detectable viral load levels as measured by PCR
Viral load reduction of >1 log or non-detectable viral load levels as measured by PBMC viral load measurements.
Stabilization or Increase of Body Mass and Body Weight.
Stabilization and Improvement of the quality of life of the patient.

Summary: This is the first double-blind, placebo controlled trial for VGV-1 in HIV-1 infected patients and has been designed and is being conducted in South Africa with guidance from regulators and HIV clinicians. Furthermore, the trials were conducted by CRO's with experience in HIV clinical trials and ICH guidelines. The study was completed in 2006. No apparent toxicities or significant adverse events related to drug were observed. 22% of subjects showed significant decrease in HIV viral load 100 days after treatment.

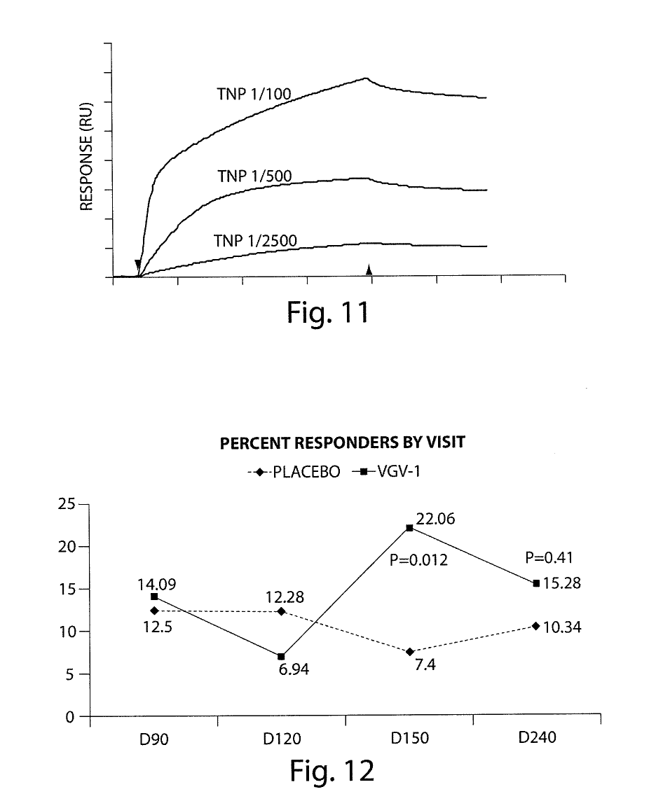

FIG. 12 is a graph showing the percentage of patients overall who had a good “response” (decrease of 0.5 log or 70% in HIV viral load) at different time points during the study compared to the patients who received placebo to those that received VGV-1. The strongest result appears at day 150—or 100 days after the completion of treatment. Afterwards, the result is diminished.

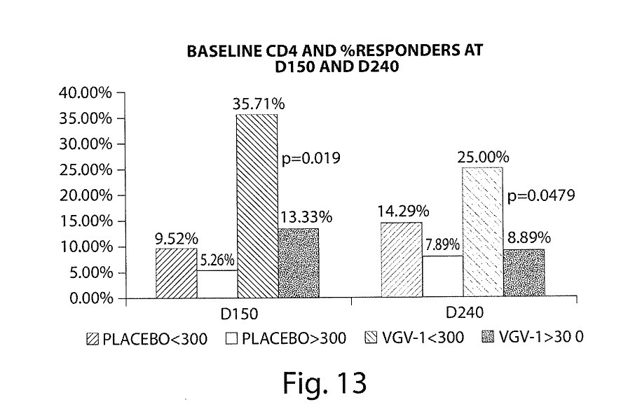

FIG. 13 is a graph showing the percentage of patients with a good “response” (decrease of 0.5 log or 70% in HIV viral load) during the study, but separates them by how strong their immune systems' were at the study's start using a measurement called CD4. Patients who were on VGV-1 with lower CD4 counts (the red columns) were much more likely to have a good response than placebo patients (orange and blue columns). Patients on VGV-1 that were healthier (grey columns) also did not do as well as these sicker patients that received VGV-1. This seems to indicate that VGV-1 works better on sicker patients than healthier patients. An explanation for these results is consistent with the model described above in Example 14.

### Example 17

### Integrated Summary of Activity and Safety Markers

A meta-analysis was conducted including subjects from all completed clinical trials, except for the 4 subjects in the first pilot Bulgarian study. It is important to note that VGV-1 monotherapy was only tested in the clinical trial conducted in China. The concomitant anti-viral therapy was not controlled for in the other clinical trials and this must be factored into the interpretation of the results.

Safety Analysis: Seven-nine (79) patients with the various CDC stage were enrolled in four studies (Tijuana, Bulgaria-2, Monterrey, and China). There were a total of 2044 adverse events (1850 in treatment and 194 in post-treatment period). Eight hundred and two (802) adverse events were digestive system related, 378 nervous system-related, 11 respiratory related, 16 urogenital related, 27 rashes skin related and 775 were body-as-a-whole related. The Fisher's exact test was used to determine if the difference of each adverse event between treatment period and post-treatment period was significant. The adverse events seen in the studies were typical of those found in patients with HIV/AIDS.

Activity: A summary from the four completed ex-US clinical trials of CD4 count and HIV-RNA levels are provided in Tables 19 to 21. The meta analysis of both viral load and CD4 cell counts from four previous clinical studies suggests that VGV-1 does not accelerate disease progression and supports the safety of VGV-1 in HIV-1 infected individuals.

Summary: Safety parameters including standard hematology and chemistry labs were collected in previous trials of VGV-1. In addition, adverse events were monitored. Finally, activity markers including CD4 cell counts and HIV-1 RNA were obtained. Overall, these data lend support to adequate safety profile for VGV-1 in HIV infected to subjects who were either naïve in anti-viral therapy or taking concomitant anti-viral medications. This meta-analysis also supports the safety of VGV-1 treatment in HIV-1 infected patients for 8 mg twice a week intra-muscular injections for 8 weeks of treatment.

TABLE 19
Baseline CD4 and HIV/RNA (log 10) for 4 Studies
Seventy-nine patients were enrolled in four studies with various CDC
stage. The mean CD4 at the baseline was 224 (stdev 232) ranged
from 7 to 1122. The mean LOG RNA at the baseline was 4.54LOG
(stdev 0.81LOG) ranged from 1.85LOG to 5.84LOG.

| Variable | N* | Median | Mean | Std Dev | Minimum | Maximum |
|---|---|---|---|---|---|---|
| CD4_BS | 79 | 139.50 | 224.1 | 231.96 | 7.0 | 1122.0 |
| RNA_BS | 79 | 4.64 | 4.54 | 0.81 | 1.86 | 5.84 |

*79 subjects were analyzed: 15 from Tijuana Mexico study, 20 from the second Bulgarian study, 10 from the Monterrey Mexico study, and 34 from the China AIDS study.

TABLE 20
Changes in CD4 Cell Count from Baseline at Different Time Points
(Months)
With the intent-to-treat population the four studies showed that mean
CD4 change from baseline was increased at month 3 (by 21),
unchanged at month 4 and increased at month 6 (by 12), but small
decreased at month 5 (by 5) and at month 9 (by 13).

| Variable | N* | Median | Mean | Std Dev | Minimum | Maximum |
|---|---|---|---|---|---|---|
| CD4_BS | 79 | 139.50 | 224.06 | 231.96 | 7.00 | 1122.00 |
| chcd4_m3 | 40 | 5.75 | 21.45 | 105.29 | −137.00 | 379.50 |
| chcd4_m4 | 54 | −3.00 | 1.0741 | 92.41 | −152.00 | 561.50 |
| chcd4_m5 | 54 | −2.00 | −4.982 | 97.28 | −468.00 | 205.50 |
| chcd4_m6 | 31 | 2.00 | 11.839 | 152.41 | −243.00 | 655.50 |
| chcd4_m9 | 34 | −7.75 | −12.838 | 84.54 | −184.50 | 167.50 |

*79 subjects were analyzed: 15 from Tijuana Mexico study, 20 from the second Bulgarian study, 10 from the Monterrey Mexico study, and 34 from the China AIDS study.

TABLE 21
Changes in Viral Load HIV/RNA (log 10) from Baseline at Different
Time Points (Months)
With the intent-to-treat population the study showed that mean LOG
RNA change from baseline was reduced at all follow-up months,
month 3 (by 0.42LOG), month 5 (by 0.52LOG) and month 6 (by
0.41LOG) and at month 9 (by 0.65LOG).

| Variable | N* | Median | Mean | Std Dev | Minimum | Maximum |
|---|---|---|---|---|---|---|
| RNA_BS | 79 | 4.640 | 4.544 | 0.807 | 1.854 | 5.838 |
| chrna_m3 | 36 | −0.134 | −0.421 | 0.884 | −2.681 | 0.800 |
| chrna_m4 | 36 | −0.007 | −0.068 | 0.725 | −2.985 | 1.360 |
| chrna_m5 | 36 | −0.335 | −0.517 | 1.128 | −3.540 | 2.297 |
| chrna_m6 | 30 | −0.332 | −0.413 | 1.024 | −2.681 | 1.190 |
| chrna_m9 | 52 | −0.455 | −0.647 | 1.199 | −4.040 | 1.730 |

*79 subjects were analyzed: 15 from Tijuana Mexico study, 20 from the second Bulgarian study, 10 from the Monterrey Mexico study, and 34 from the China AIDS study.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A composition comprising
a thymus derived peptide and a pharmaceutically acceptable carrier, wherein the composition is not a thymus nuclear protein extract.

2. The composition of claim 1, wherein the thymus derived peptide is synthetic.

3. The composition of claim 1, wherein the thymus derived peptide is a peptide of any of SEQ ID NO. 1-SEQ ID NO. 265.

4. The composition of claim 1, wherein the thymus derived peptide is isolated.

5. The composition of claim 1, wherein the composition is free of cystatin A protein or a histone protein.

6. The composition of claim 1, further comprising an adjuvant.

7. The composition of claim 1, wherein the composition has a binding affinity for gp120 of at least 5000 RD.

8. (canceled)

9. The composition of claim 1, wherein the thymus derived peptide is a peptide of any of SEQ ID NO. 49, 58, 59, 61, 62, 66, 67, 68, 69, 76, 77, 78, 81, 82, 86, 89, 90, 92, 104, 109, 110, 112, 117, 128, 129, 133, 136, 140, 141, 144, 146, 148, 149, 150, 154, 156, 157, 161, 162, 164, 168, 171, 172, 175, 177, 179, 186, 187, 188, 190, 191, 192, 196, 197, 201, 204, 205, 210, 217, 218, 220, 221, 222, 226, and 227.

10-13. (canceled)

14. The composition of claim 1, further comprising an anti-HIV agent.

15. The composition of claim 1, wherein the composition is formulated for oral administration, intranasal administration, or pulmonary administration.

16-17. (canceled)

18. The composition of claim 1, wherein the composition includes 1 thymus derived peptide.

19. The composition of claim 1, wherein the composition includes 2-100 thymus derived peptides.

20. The composition of claim 1, further comprising an antigen.

21. The composition of claim 1, further comprising an anti-cancer agent.

22. The composition of claim 1, further comprising an Alzheimer's medicament.

23. A method of treatment for HIV infection comprising administering to a human infected with HIV or at risk of HIV infection a composition comprising a thymus derived peptide and a pharmaceutically acceptable carrier, wherein the composition does not include every peptide of a thymus nuclear protein extract.

24-28. (canceled)

29. The method according to claim 23, wherein the thymus derived peptide is a peptide of any of SEQ ID NO. 49, 58, 59, 61, 62, 66, 67, 68, 69, 76, 77, 78, 81, 82, 86, 89, 90, 92, 104, 109, 110, 112, 117, 128, 129, 133, 136, 140, 141, 144, 146, 148, 149, 150, 154, 156, 157, 161, 162, 164, 168, 171, 172, 175, 177, 179, 186, 187, 188, 190, 191, 192, 196, 197, 201, 204, 205, 210, 217, 218, 220, 221, 222, 226, and 227.

30-34. (canceled)

35. A method for diagnosing HIV infection comprising
(a) collecting a sample from a subject;
(b) mixing the sample with a thymus derived peptide that is not cystatin A protein or a histone protein; and
(c) identifying a complex of the thymus derived peptide bound to CD4, gp120 or gp41, wherein the complex is indicative of HIV-I infection.

36-40. (canceled)

41. A kit for detection of HIV comprising
(a) a container housing a thymus derived peptide that is not cystatin A protein or a histone protein;
(b) a reagent for identifying at least one complex of said cystatin A protein and said at least one histone protein with CD4, gp120 or gp41; and
(c) instructions for identifying a complex that is indicative of HIV-I infection.

42. A kit comprising
(a) a container housing a thymus derived peptide wherein the thymus derived peptide is a peptide of any of SEQ ID NO. 1-SEQ ID NO. 265; and
(b) instructions for identifying a complex that is indicative of HIV-I infection.

43. A method of treatment for disease associated with a decrease in the number of T_{H} cells comprising administering to a subject in need thereof a composition comprising a thymus derived peptide and a pharmaceutically acceptable carrier, wherein the composition is not a thymus nuclear protein extract, in an effective amount to treat the disease.

44. The method according to claim 43, wherein the disease is an autoimmune disease selected from the group consisting of multiple sclerosis, rheumatoid arthritis, dermatitis, type 1 diabetes mellitus, colitis, inflammatory bowel disease/irritable bowel syndrome, Crohn's disease, Psoriasis, and Systemic lupus erythematosus.

45-47. (canceled)

48. A method for treating cancer, comprising:
administering to a subject having cancer a composition comprising a thymus derived peptide and a pharmaceutically acceptable carrier, wherein the composition is not a thymus nuclear protein extract, in an effective amount to treat the cancer in the subject.

49-52. (canceled)

53. A method for treating a viral infection, comprising:
administering to a subject having or at risk of having a viral infection a composition comprising a thymus derived peptide and a pharmaceutically acceptable carrier, wherein the composition is not a thymus nuclear protein extract, in an effective amount to treat the viral infection in the subject.

54-59. (canceled)

60. The method of claim 53, wherein the thymus derived peptide is a peptide of any of SEQ ID NO. 49, 58, 59, 61, 62, 66, 67, 68, 69, 76, 77, 78, 81, 82, 86, 89, 90, 92, 104, 109, 110, 112, 117, 128, 129, 133, 136, 140, 141, 144, 146, 148, 149, 150, 154, 156, 157, 161, 162, 164, 168, 171, 172, 175, 177, 179, 186, 187, 188, 190, 191, 192, 196, 197, 201, 204, 205, 210, 217, 218, 220, 221, 222, 226, and 227.

61. A method for treating a subject having a cell or tissue graft, comprising:
administering to the subject in need thereof composition comprising a thymus derived peptide and a pharmaceutically acceptable carrier, wherein the composition is not a thymus nuclear protein extract, in an effective amount to inhibit cell or tissue graft rejection in the subject.

62-63. (canceled)

64. A peptide having a peptide sequence corresponding to any one of SEQ ID NOs. 1-265.

65. A method of treating HIV, comprising administering to a subject a peptide of claim 64.

66. A method of treating a disease associated with a decrease in the number of TH cells comprising administering to a subject in need thereof a peptide of claim 64.

67. A method of treating cancer comprising administering to a subject in need thereof a peptide of claim 64.

68. A method of treating a subject having a cell or tissue graft comprising administering to a subject in need thereof a peptide of claim 64.


SEQUENCE LISTING (270 sequences)

SEQ ID NO: 1 - protein, 14 aa, Artificial Sequence
  Synthetic Peptide
KALVQNDTLLQVKG

SEQ ID NO: 2 - protein, 17 aa, Artificial Sequence
  Synthetic Peptide
KAMDIMNSFVNDIFERI

SEQ ID NO: 3 - protein, 17 aa, Artificial Sequence
  Synthetic Peptide
KAMGIMKSFVNDIFERI

SEQ ID NO: 4 - protein, 17 aa, Artificial Sequence
  Synthetic Peptide
KAMGNMNSFVNDIFERI

SEQ ID NO: 5 - protein, 17 aa, Artificial Sequence
  Synthetic Peptide
KAMSIMNSFVNDLFERL

SEQ ID NO: 6 - protein, 14 aa, Artificial Sequence
  Synthetic Peptide
KASGPPVSELITKA

SEQ ID NO: 7 - protein, 15 aa, Artificial Sequence
  Synthetic Peptide
KDAFLGSFLYEYSRR

SEQ ID NO: 8 - protein, 15 aa, Artificial Sequence
  Synthetic Peptide
KDDPHACYSTVFDKL

SEQ ID NO: 9 - protein, 18 aa, Artificial Sequence
  Synthetic Peptide
KEFFQSAIKLVDFQDAKA

SEQ ID NO: 10 - protein, 11 aa, Artificial Sequence
  Synthetic Peptide
KESYSVYVYKV

SEQ ID NO: 11 - protein, 23 aa, Artificial Sequence
  Synthetic Peptide
KGLVLIAFSQYLQQCPFDEHVKL

SEQ ID NO: 12 - protein, 13 aa, Artificial Sequence
  Synthetic Peptide
KHLVDEPQNLIKQ

SEQ ID NO: 13 - protein, 15 aa, Artificial Sequence
  Synthetic Peptide
KHPDSSVNFAEFSKK

SEQ ID NO: 14 - protein, 12 aa, Artificial Sequence
  Synthetic Peptide
KKQTALVELLKH

SEQ ID NO: 15 - protein, 17 aa, Artificial Sequence
  Synthetic Peptide
KKVPEVSTPTLVEVSRN

SEQ ID NO: 16 - protein, 18 aa, Artificial Sequence
  Synthetic Peptide
KLFTFHADICTLPDTEKQ

SEQ ID NO: 17 - protein, 15 aa, Artificial Sequence
  Synthetic Peptide
KLGEYGFQNALIVRY

SEQ ID NO: 18 - protein, 15 aa, Artificial Sequence
  Synthetic Peptide
KLKPDPNTLCDEFKA

SEQ ID NO: 19 - protein, 12 aa, Artificial Sequence
  Synthetic Peptide
KLVNELTEFAKT

SEQ ID NO: 20 - protein, 11 aa, Artificial Sequence
  Synthetic Peptide
KLVVSTQTALA

SEQ ID NO: 21 - protein, 11 aa, Artificial Sequence
  Synthetic Peptide
KQTALVELLKH

SEQ ID NO: 22 - protein, 14 aa, Artificial Sequence
  Synthetic Peptide
KSLHTLFGDELCKV

SEQ ID NO: 23 - protein, 18 aa, Artificial Sequence
  Synthetic Peptide
KTITLEVEPSDTIENVKA

SEQ ID NO: 24 - protein, 14 aa, Artificial Sequence
  Synthetic Peptide
KTVMENFVAFVDKC

SEQ ID NO: 25 - protein, 31 aa, Artificial Sequence
  Synthetic Peptide
KTVMENFVAFVDKCCAADDKEACFAVEGPKL

SEQ ID NO: 26 - protein, 14 aa, Artificial Sequence
  Synthetic Peptide
KTVTAMDVVYALKR

SEQ ID NO: 27 - protein, 10 aa, Artificial Sequence
  Synthetic Peptide
KVFLENVIRD

SEQ ID NO: 28 - protein, 16 aa, Artificial Sequence
  Synthetic Peptide
KVPEVSTPTLVEVSRN

SEQ ID NO: 29 - protein, 9 aa, Artificial Sequence
  Synthetic Peptide
KYLYEIARR

SEQ ID NO: 30 - protein, 15 aa, Artificial Sequence
  Synthetic Peptide
MGIMNSFVNDIFERI

SEQ ID NO: 31 - protein, 11 aa, Artificial Sequence
  Synthetic Peptide
RAGLQFPVGRV

SEQ ID NO: 32 - protein, 14 aa, Artificial Sequence
  Synthetic Peptide
RDNIQGITKPAIRR

SEQ ID NO: 33 - protein, 13 aa, Artificial Sequence
  Synthetic Peptide
REIAQDFKTDLRF

SEQ ID NO: 34 - protein, 34 aa, Artificial Sequence
  Synthetic Peptide
RFQSAAIGALQEASEAYLVGLFEDTNLCAIHAKR

SEQ ID NO: 35 - protein, 12 aa, Artificial Sequence
  Synthetic Peptide
RILGLIYEETRR

SEQ ID NO: 36 - protein, 12 aa, Artificial Sequence
  Synthetic Peptide
RISGLIYEETRG

SEQ ID NO: 37 - protein, 12 aa, Artificial Sequence
  Synthetic Peptide
RISGLIYKETRR

SEQ ID NO: 38 - protein, 12 aa, Artificial Sequence
  Synthetic Peptide
RKENHSVYVYKV

SEQ ID NO: 39 - protein, 11 aa, Artificial Sequence
  Synthetic Peptide
RLLLPGELAKH

SEQ ID NO: 40 - protein, 13 aa, Artificial Sequence
  Synthetic Peptide
RNDEELNKLLGKV

SEQ ID NO: 41 - protein, 18 aa, Artificial Sequence
  Synthetic Peptide
RNECFLSHKDDSPDLPKL

SEQ ID NO: 42 - protein, 18 aa, Artificial Sequence
  Synthetic Peptide
RRPCFSALTPDETYVPKA

SEQ ID NO: 43 - protein, 8 aa, Artificial Sequence
  Synthetic Peptide
RTLYGFGG

SEQ ID NO: 44 - protein, 17 aa, Artificial Sequence
  Synthetic Peptide
RTSKLQNEIDVSSREKS

SEQ ID NO: 45 - protein, 21 aa, Artificial Sequence
  Synthetic Peptide
RVTIAQGGVLPNIQAVLLPKK

SEQ ID NO: 46 - protein, 9 aa, Artificial Sequence
  Synthetic Peptide
LPDTEKQKL

SEQ ID NO: 47 - protein, 9 aa, Artificial Sequence
  Synthetic Peptide
YSTVFDKLK

SEQ ID NO: 48 - protein, 9 aa, Artificial Sequence
  Synthetic Peptide
ITLEVEPSD

SEQ ID NO: 49 - protein, 9 aa, Artificial Sequence
  Synthetic Peptide
LVQNDTLLQ

SEQ ID NO: 50 - protein, 9 aa, Artificial Sequence
  Synthetic Peptide
IKAMGIMKS

SEQ ID NO: 51 - protein, 9 aa, Artificial Sequence
  Synthetic Peptide
IKAMSIMNS

SEQ ID NO: 52 - protein, 9 aa, Artificial Sequence
  Synthetic Peptide
YVYKVRLLL

SEQ ID NO: 53 - protein, 9 aa, Artificial Sequence
  Synthetic Peptide
IKAMGNMNS

SEQ ID NO: 54 - protein, 9 aa, Artificial Sequence
  Synthetic Peptide
VRLLLPGEL

SEQ ID NO: 55 - protein, 9 aa, Artificial Sequence
  Synthetic Peptide
VVYALKRKV

SEQ ID NO: 56 - protein, 9 aa, Artificial Sequence
  Synthetic Peptide
YEIARRMGI

SEQ ID NO: 57 - protein, 9 aa, Artificial Sequence
  Synthetic Peptide
FRFQSAAIG

SEQ ID NO: 58 - protein, 9 aa, Artificial Sequence
  Synthetic Peptide
VVSTQTALA

SEQ ID NO: 59 - protein, 9 aa, Artificial Sequence
  Synthetic Peptide
IMNSFVNDI

SEQ ID NO: 60 - protein, 9 aa, Artificial Sequence
  Synthetic Peptide
ICTLPDTEK

SEQ ID NO: 61 - protein, 9 aa, Artificial Sequence
  Synthetic Peptide
MGIMKSFVN

SEQ ID NO: 62 - protein, 9 aa, Artificial Sequence
  Synthetic Peptide
MGIMNSFVN

SEQ ID NO: 63 - protein, 9 aa, Artificial Sequence
  Synthetic Peptide
LVELLKHKS

SEQ ID NO: 64 - protein, 9 aa, Artificial Sequence
  Synthetic Peptide
FERIKAMGI

SEQ ID NO: 65 - protein, 9 aa, Artificial Sequence
  Synthetic Peptide
FERIKAMSI

SEQ ID NO: 66 - protein, 9 aa, Artificial Sequence
  Synthetic Peptide
VLIAFSQYL

SEQ ID NO: 67 - protein, 9 aa, Artificial Sequence
  Synthetic Peptide
IMNSFVNDL

SEQ ID NO: 68 - protein, 9 aa, Artificial Sequence
  Synthetic Peptide
IMKSFVNDI

SEQ ID NO: 69 - protein, 9 aa, Artificial Sequence
  Synthetic Peptide
IQGITKPAI

SEQ ID NO: 70 - protein, 9 aa, Artificial Sequence
  Synthetic Peptide
VYVYKVRLL

SEQ ID NO: 71 - protein, 9 aa, Artificial Sequence
  Synthetic Peptide
YVYKVKGLV

SEQ ID NO: 72 - protein, 9 aa, Artificial Sequence
  Synthetic Peptide
LIYKETRRR

SEQ ID NO: 73 - protein, 9 aa, Artificial Sequence
  Synthetic Peptide
VKGLVLIAF

SEQ ID NO: 74 - protein, 9 aa, Artificial Sequence
  Synthetic Peptide
IRRREIAQD

SEQ ID NO: 75 - protein, 9 aa, Artificial Sequence
  Synthetic Peptide
VYVYKVKGL

SEQ ID NO: 76 - protein, 9 aa, Artificial Sequence
  Synthetic Peptide
VTAMDVVYA

SEQ ID NO: 77 - protein, 9 aa, Artificial Sequence
  Synthetic Peptide
YGFQNALIV

SEQ ID NO: 78 - protein, 9 aa, Artificial Sequence
  Synthetic Peptide
LVNELTEFA

SEQ ID NO: 79 - protein, 9 aa, Artificial Sequence
  Synthetic Peptide
VRYKLKPDP

SEQ ID NO: 80 - protein, 9 aa, Artificial Sequence
  Synthetic Peptide
LKTVTAMDV

SEQ ID NO: 81 - protein, 9 aa, Artificial Sequence
  Synthetic Peptide
FQNALIVRY

SEQ ID NO: 82 - protein, 9 aa, Artificial Sequence
  Synthetic Peptide
MSIMNSFVN

SEQ ID NO: 83 - protein, 9 aa, Artificial Sequence
  Synthetic Peptide
VKAKTVMEN

SEQ ID NO: 84 - protein, 9 aa, Artificial Sequence
  Synthetic Peptide
FKAKLVNEL

SEQ ID NO: 85 - protein, 9 aa, Artificial Sequence
  Synthetic Peptide
LRFRFQSAA

SEQ ID NO: 86 - protein, 9 aa, Artificial Sequence
  Synthetic Peptide
LVLIAFSQY

SEQ ID NO: 87 - protein, 9 aa, Artificial Sequence
  Synthetic Peptide
LKASGPPVS

SEQ ID NO: 88 - protein, 9 aa, Artificial Sequence
  Synthetic Peptide
VIRDKVPEV

SEQ ID NO: 89 - protein, 9 aa, Artificial Sequence
  Synthetic Peptide
VQNDTLLQV

SEQ ID NO: 90 - protein, 9 aa, Artificial Sequence
  Synthetic Peptide
MGNMNSFVN

SEQ ID NO: 91 - protein, 9 aa, Artificial Sequence
  Synthetic Peptide
YVPKARTLY

SEQ ID NO: 92 - protein, 9 aa, Artificial Sequence
  Synthetic Peptide
FQSAIKLVD

SEQ ID NO: 93 - protein, 9 aa, Artificial Sequence
  Synthetic Peptide
LYGFGGRTS

SEQ ID NO: 94 - protein, 9 aa, Artificial Sequence
  Synthetic Peptide
YKVKGLVLI

SEQ ID NO: 95 - protein, 9 aa, Artificial Sequence
  Synthetic Peptide
LVELLKHKK

SEQ ID NO: 96 - protein, 9 aa, Artificial Sequence
  Synthetic Peptide
LKHKKVPEV

SEQ ID NO: 97 - protein, 9 aa, Artificial Sequence
  Synthetic Peptide
LLKHKSLHT

SEQ ID NO: 98 - protein, 9 aa, Artificial Sequence
  Synthetic Peptide
YKVRLLLPG

SEQ ID NO: 99 - protein, 9 aa, Artificial Sequence
  Synthetic Peptide
VRNECFLSH

SEQ ID NO: 100 - protein, 9 aa, Artificial Sequence
  Synthetic Peptide
IVRYKLKPD

SEQ ID NO: 101 - protein, 9 aa, Artificial Sequence
  Synthetic Peptide
LIVRYKLKP

SEQ ID NO: 102 - protein, 9 aa, Artificial Sequence
  Synthetic Peptide
LLGKVRNEC

SEQ ID NO: 103 - protein, 9 aa, Artificial Sequence
  Synthetic Peptide
FERIKAMGN

SEQ ID NO: 104 - protein, 9 aa, Artificial Sequence
  Synthetic Peptide
VAFVDKCCA

SEQ ID NO: 105 - protein, 9 aa, Artificial Sequence
  Synthetic Peptide
LIYEETRRR

SEQ ID NO: 106 - protein, 9 aa, Artificial Sequence
  Synthetic Peptide
LIYEETRGR

SEQ ID NO: 107 - protein, 9 aa, Artificial Sequence
  Synthetic Peptide
VYALKRKVF

SEQ ID NO: 108 - protein, 9 aa, Artificial Sequence
  Synthetic Peptide
YLYEIARRM

SEQ ID NO: 109 - protein, 9 aa, Artificial Sequence
  Synthetic Peptide
LVVSTQTAL

SEQ ID NO: 110 - protein, 9 aa, Artificial Sequence
  Synthetic Peptide
VFLENVIRD

SEQ ID NO: 111 - protein, 9 aa, Artificial Sequence
  Synthetic Peptide
LVEVSRNKL

SEQ ID NO: 112 - protein, 9 aa, Artificial Sequence
  Synthetic Peptide
LIAFSQYLQ

SEQ ID NO: 113 - protein, 9 aa, Artificial Sequence
  Synthetic Peptide
IRDKVPEVS

SEQ ID NO: 114 - protein, 9 aa, Artificial Sequence
  Synthetic Peptide
LCKVKTITL

SEQ ID NO: 115 - protein, 9 aa, Artificial Sequence
  Synthetic Peptide
LIKQKHPDS

SEQ ID NO: 116 - protein, 9 aa, Artificial Sequence
  Synthetic Peptide
FERIRAGLQ

SEQ ID NO: 117 - protein, 9 aa, Artificial Sequence
  Synthetic Peptide
FQSAAIGAL

SEQ ID NO: 118 - protein, 9 aa, Artificial Sequence
  Synthetic Peptide
LVEVSRNKY

SEQ ID NO: 119 - protein, 9 aa, Artificial Sequence
  Synthetic Peptide
VKLKHLVDE

SEQ ID NO: 120 - protein, 9 aa, Artificial Sequence
  Synthetic Peptide
VYKVKGLVL

SEQ ID NO: 121 - protein, 9 aa, Artificial Sequence
  Synthetic Peptide
YALKRKVFL

SEQ ID NO: 122 - protein, 9 aa, Artificial Sequence
  Synthetic Peptide
VELLKHKKV

SEQ ID NO: 123 - protein, 9 aa, Artificial Sequence
  Synthetic Peptide
LQVKGKAMD

SEQ ID NO: 124 - protein, 9 aa, Artificial Sequence
  Synthetic Peptide
LKHKSLHTL

SEQ ID NO: 125 - protein, 9 aa, Artificial Sequence
  Synthetic Peptide
VELLKHKSL

SEQ ID NO: 126 - protein, 9 aa, Artificial Sequence
  Synthetic Peptide
VPKARTLYG

SEQ ID NO: 127 - protein, 9 aa, Artificial Sequence
  Synthetic Peptide
FKTDLRFRF

SEQ ID NO: 128 - protein, 9 aa, Artificial Sequence
  Synthetic Peptide
MDIMNSFVN

SEQ ID NO: 129 - protein, 9 aa, Artificial Sequence
  Synthetic Peptide
IKLVDFQDA

SEQ ID NO: 130 - protein, 9 aa, Artificial Sequence
  Synthetic Peptide
FVDKCKTVM

SEQ ID NO: 131 - protein, 9 aa, Artificial Sequence
  Synthetic Peptide
IHAKRRILG

SEQ ID NO: 132 - protein, 9 aa, Artificial Sequence
  Synthetic Peptide
FLYEYSRRK

SEQ ID NO: 133 - protein, 9 aa, Artificial Sequence
  Synthetic Peptide
VMENFVAFV

SEQ ID NO: 134 - protein, 9 aa, Artificial Sequence
  Synthetic Peptide
YLVGLFEDT

SEQ ID NO: 135 - protein, 9 aa, Artificial Sequence
  Synthetic Peptide
VYKVRLLLP

SEQ ID NO: 136 - protein, 9 aa, Artificial Sequence
  Synthetic Peptide
YLQQCPFDE

SEQ ID NO: 137 - protein, 9 aa, Artificial Sequence
  Synthetic Peptide
IRAGLQFPV

SEQ ID NO: 138 - protein, 9 aa, Artificial Sequence
  Synthetic Peptide
LLKHKKVPE

SEQ ID NO: 139 - protein, 9 aa, Artificial Sequence
  Synthetic Peptide
IKQKHPDSS

SEQ ID NO: 140 - protein, 9 aa, Artificial Sequence
  Synthetic Peptide
VLPNIQAVL

SEQ ID NO: 141 - protein, 9 aa, Artificial Sequence
  Synthetic Peptide
VEPSDTIEN

SEQ ID NO: 142 - protein, 9 aa, Artificial Sequence
  Synthetic Peptide
FGGRTSKLQ

SEQ ID NO: 143 - protein, 9 aa, Artificial Sequence
  Synthetic Peptide
VAFVDKCKT

SEQ ID NO: 144 - protein, 9 aa, Artificial Sequence
  Synthetic Peptide
FFQSAIKLV

SEQ ID NO: 145 - protein, 9 aa, Artificial Sequence
  Synthetic Peptide
FQDAKAKES

SEQ ID NO: 146 - protein, 9 aa, Artificial Sequence
  Synthetic Peptide
IQAVLLPKK

SEQ ID NO: 147 - protein, 9 aa, Artificial Sequence
  Synthetic Peptide
LLQVKGKAM

SEQ ID NO: 148 - protein, 9 aa, Artificial Sequence
  Synthetic Peptide
IAFSQYLQQ

SEQ ID NO: 149 - protein, 9 aa, Artificial Sequence
  Synthetic Peptide
FLGSFLYEY

SEQ ID NO: 150 - protein, 9 aa, Artificial Sequence
  Synthetic Peptide
FVNDIFERI

SEQ ID NO: 151 - protein, 9 aa, Artificial Sequence
  Synthetic Peptide
VDEPQNLIK

SEQ ID NO: 152 - protein, 9 aa, Artificial Sequence
  Synthetic Peptide
LSHKDDSPD

SEQ ID NO: 153 - protein, 9 aa, Artificial Sequence
  Synthetic Peptide
FLSHKDDSP

SEQ ID NO: 154 - protein, 9 aa, Artificial Sequence
  Synthetic Peptide
LPNIQAVLL

SEQ ID NO: 155 - protein, 9 aa, Artificial Sequence
  Synthetic Peptide
LKRKVFLEN

SEQ ID NO: 156 - protein, 9 aa, Artificial Sequence
  Synthetic Peptide
LLPGELAKH

SEQ ID NO: 157 - protein, 9 aa, Artificial Sequence
  Synthetic Peptide
FVAFVDKCC

SEQ ID NO: 158 - protein, 9 aa, Artificial Sequence
  Synthetic Peptide
IFERIKAMS

SEQ ID NO: 159 - protein, 9 aa, Artificial Sequence
  Synthetic Peptide
IENVKAKTV

SEQ ID NO: 160 - protein, 9 aa, Artificial Sequence
  Synthetic Peptide
VSRNKLFTF

SEQ ID NO: 161 - protein, 9 aa, Artificial Sequence
  Synthetic Peptide
LKPDPNTLC

SEQ ID NO: 162 - protein, 9 aa, Artificial Sequence
  Synthetic Peptide
MENFVAFVD

SEQ ID NO: 163 - protein, 9 aa, Artificial Sequence
  Synthetic Peptide
YSRRKDDPH

SEQ ID NO: 164 - protein, 9 aa, Artificial Sequence
  Synthetic Peptide
LFGDELCKV

SEQ ID NO: 165 - protein, 9 aa, Artificial Sequence
  Synthetic Peptide
FERLKASGP

SEQ ID NO: 166 - protein, 9 aa, Artificial Sequence
  Synthetic Peptide
VSTQTALAK

SEQ ID NO: 167 - protein, 9 aa, Artificial Sequence
  Synthetic Peptide
FAKTKLVVS

SEQ ID NO: 168 - protein, 9 aa, Artificial Sequence
  Synthetic Peptide
VTIAQGGVL

SEQ ID NO: 169 - protein, 9 aa, Artificial Sequence
  Synthetic Peptide
LNKLLGKVR

SEQ ID NO: 170 - protein, 9 aa, Artificial Sequence
  Synthetic Peptide
LYEIARRMG

SEQ ID NO: 171 - protein, 9 aa, Artificial Sequence
  Synthetic Peptide
MKSFVNDIF

SEQ ID NO: 172 - protein, 9 aa, Artificial Sequence
  Synthetic Peptide
LFTFHADIC

SEQ ID NO: 173 - protein, 9 aa, Artificial Sequence
  Synthetic Peptide
LAKQTALVE

SEQ ID NO: 174 - protein, 9 aa, Artificial Sequence
  Synthetic Peptide
FVAFVDKCK

SEQ ID NO: 175 - protein, 9 aa, Artificial Sequence
  Synthetic Peptide
FVNDLFERL

SEQ ID NO: 176 - protein, 9 aa, Artificial Sequence
  Synthetic Peptide
VKTITLEVE

SEQ ID NO: 177 - protein, 9 aa, Artificial Sequence
  Synthetic Peptide
IAQGGVLPN

SEQ ID NO: 178 - protein, 9 aa, Artificial Sequence
  Synthetic Peptide
LRRPCFSAL

SEQ ID NO: 179 - protein, 9 aa, Artificial Sequence
  Synthetic Peptide
LGSFLYEYS

SEQ ID NO: 180 - protein, 9 aa, Artificial Sequence
  Synthetic Peptide
LCAIHAKRR

SEQ ID NO: 181 - protein, 9 aa, Artificial Sequence
  Synthetic Peptide
LPKLRRPCF

SEQ ID NO: 182 - protein, 9 aa, Artificial Sequence
  Synthetic Peptide
VEVSRNKLF

SEQ ID NO: 183 - protein, 9 aa, Artificial Sequence
  Synthetic Peptide
FLENVIRDK

SEQ ID NO: 184 - protein, 9 aa, Artificial Sequence
  Synthetic Peptide
IYKETRRRK

SEQ ID NO: 185 - protein, 9 aa, Artificial Sequence
  Synthetic Peptide
VEVSRNKYL

SEQ ID NO: 186 - protein, 9 aa, Artificial Sequence
  Synthetic Peptide
FVDKCCAAD

SEQ ID NO: 187 - protein, 9 aa, Artificial Sequence
  Synthetic Peptide
LFEDTNLCA

SEQ ID NO: 188 - protein, 9 aa, Artificial Sequence
  Synthetic Peptide
VNFAEFSKK

SEQ ID NO: 189 - protein, 9 aa, Artificial Sequence
  Synthetic Peptide
VGRVRDNIQ

SEQ ID NO: 190 - protein, 9 aa, Artificial Sequence
  Synthetic Peptide
MNSFVNDIF

SEQ ID NO: 191 - protein, 9 aa, Artificial Sequence
  Synthetic Peptide
MNSFVNDLF

SEQ ID NO: 192 - protein, 9 aa, Artificial Sequence
  Synthetic Peptide
LVDEPQNLI

SEQ ID NO: 193 - protein, 9 aa, Artificial Sequence
  Synthetic Peptide
FSKKKKQTA

SEQ ID NO: 194 - protein, 9 aa, Artificial Sequence
  Synthetic Peptide
YGFGGRTSK

SEQ ID NO: 195 - protein, 9 aa, Artificial Sequence
  Synthetic Peptide
LITKAKDAF

SEQ ID NO: 196 - protein, 9 aa, Artificial Sequence
  Synthetic Peptide
MDVVYALKR

SEQ ID NO: 197 - protein, 9 aa, Artificial Sequence
  Synthetic Peptide
LLLPGELAK

SEQ ID NO: 198 - protein, 9 aa, Artificial Sequence
  Synthetic Peptide
LQFPVGRVR

SEQ ID NO: 199 - protein, 9 aa, Artificial Sequence
  Synthetic Peptide
LKEFFQSAI

SEQ ID NO: 200 - protein, 9 aa, Artificial Sequence
  Synthetic Peptide
YEYSRRKDD

SEQ ID NO: 201 - protein, 9 aa, Artificial Sequence
  Synthetic Peptide
LTPDETYVP

SEQ ID NO: 202 - protein, 9 aa, Artificial Sequence
  Synthetic Peptide
LGKVRNECF

SEQ ID NO: 203 - protein, 9 aa, Artificial Sequence
  Synthetic Peptide
LKHLVDEPQ

SEQ ID NO: 204 - protein, 9 aa, Artificial Sequence
  Synthetic Peptide
LQNEIDVSS

SEQ ID NO: 205 - protein, 9 aa, Artificial Sequence
  Synthetic Peptide
LVDFQDAKA

SEQ ID NO: 206 - protein, 9 aa, Artificial Sequence
  Synthetic Peptide
FAVEGPKLK

SEQ ID NO: 207 - protein, 9 aa, Artificial Sequence
  Synthetic Peptide
VSELITKAK

SEQ ID NO: 208 - protein, 9 aa, Artificial Sequence
  Synthetic Peptide
IFERIRAGL

SEQ ID NO: 209 - protein, 9 aa, Artificial Sequence
  Synthetic Peptide
LENVIRDKV

SEQ ID NO: 210 - protein, 9 aa, Artificial Sequence
  Synthetic Peptide
VGLFEDTNL

SEQ ID NO: 211 - protein, 9 aa, Artificial Sequence
  Synthetic Peptide
VSSREKSRV

SEQ ID NO: 212 - protein, 9 aa, Artificial Sequence
  Synthetic Peptide
IYEETRRRI

SEQ ID NO: 213 - protein, 9 aa, Artificial Sequence
  Synthetic Peptide
IFERIKAMG

SEQ ID NO: 214 - protein, 9 aa, Artificial Sequence
  Synthetic Peptide
FGDELCKVK

SEQ ID NO: 215 - protein, 9 aa, Artificial Sequence
  Synthetic Peptide
LFERLKASG

SEQ ID NO: 216 - protein, 9 aa, Artificial Sequence
  Synthetic Peptide
IARRMGIMN

SEQ ID NO: 217 - protein, 9 aa, Artificial Sequence
  Synthetic Peptide
LGLIYEETR

SEQ ID NO: 218 - protein, 9 aa, Artificial Sequence
  Synthetic Peptide
ILGLIYEET

SEQ ID NO: 219 - protein, 9 aa, Artificial Sequence
  Synthetic Peptide
YEETRRRIS

SEQ ID NO: 220 - protein, 9 aa, Artificial Sequence
  Synthetic Peptide
IDVSSREKS

SEQ ID NO: 221 - protein, 9 aa, Artificial Sequence
  Synthetic Peptide
LHTLFGDEL

SEQ ID NO: 222 - protein, 9 aa, Artificial Sequence
  Synthetic Peptide
LVGLFEDTN

SEQ ID NO: 223 - protein, 9 aa, Artificial Sequence
  Synthetic Peptide
VKGKAMDIM

SEQ ID NO: 224 - protein, 9 aa, Artificial Sequence
  Synthetic Peptide
FPVGRVRDN

SEQ ID NO: 225 - protein, 9 aa, Artificial Sequence
  Synthetic Peptide
VSRNKYLYE

SEQ ID NO: 226 - protein, 9 aa, Artificial Sequence
  Synthetic Peptide
IAQDFKTDL

SEQ ID NO: 227 - protein, 9 aa, Artificial Sequence
  Synthetic Peptide
FHADICTLP

SEQ ID NO: 228 - protein, 9 aa, Artificial Sequence
  Synthetic Peptide
VRDNIQGIT

SEQ ID NO: 229 - protein, 9 aa, Artificial Sequence
  Synthetic Peptide
YKLKPDPNT

SEQ ID NO: 230 - protein, 9 aa, Artificial Sequence
  Synthetic Peptide
VDFQDAKAK

SEQ ID NO: 231 - protein, 9 aa, Artificial Sequence
  Synthetic Peptide
FAEFSKKKK

SEQ ID NO: 232 - protein, 9 aa, Artificial Sequence
  Synthetic Peptide
LYEYSRRKD

SEQ ID NO: 233 - protein, 9 aa, Artificial Sequence
  Synthetic Peptide
FDEHVKLKH

SEQ ID NO: 234 - protein, 9 aa, Artificial Sequence
  Synthetic Peptide
LTEFAKTKL

SEQ ID NO: 235 - protein, 9 aa, Artificial Sequence
  Synthetic Peptide
LQQCPFDEH

SEQ ID NO: 236 - protein, 9 aa, Artificial Sequence
  Synthetic Peptide
LEVEPSDTI

SEQ ID NO: 237 - protein, 9 aa, Artificial Sequence
  Synthetic Peptide
IGALQEASE

SEQ ID NO: 238 - protein, 9 aa, Artificial Sequence
  Synthetic Peptide
VDKCKTVME

SEQ ID NO: 239 - protein, 9 aa, Artificial Sequence
  Synthetic Peptide
VFDKLKEFF

SEQ ID NO: 240 - protein, 9 aa, Artificial Sequence
  Synthetic Peptide
FTFHADICT

SEQ ID NO: 241 - protein, 9 aa, Artificial Sequence
  Synthetic Peptide
VPEVSTPTL

SEQ ID NO: 242 - protein, 9 aa, Artificial Sequence
  Synthetic Peptide
FSALTPDET

SEQ ID NO: 243 - protein, 9 aa, Artificial Sequence
  Synthetic Peptide
ITKPAIRRR

SEQ ID NO: 244 - protein, 9 aa, Artificial Sequence
  Synthetic Peptide
YKETRRRKE

SEQ ID NO: 245 - protein, 9 aa, Artificial Sequence
  Synthetic Peptide
IYEETRGRI

SEQ ID NO: 246 - protein, 9 aa, Artificial Sequence
  Synthetic Peptide
VEGPKLKTV

SEQ ID NO: 247 - protein, 9 aa, Artificial Sequence
  Synthetic Peptide
FEDTNLCAI

SEQ ID NO: 248 - protein, 9 aa, Artificial Sequence
  Synthetic Peptide
VNELTEFAK

SEQ ID NO: 249 - protein, 9 aa, Artificial Sequence
  Synthetic Peptide
YSVYVYKVK

SEQ ID NO: 250 - protein, 9 aa, Artificial Sequence
  Synthetic Peptide
LQEASEAYL

SEQ ID NO: 251 - protein, 9 aa, Artificial Sequence
  Synthetic Peptide
ISGLIYKET

SEQ ID NO: 252 - protein, 9 aa, Artificial Sequence
  Synthetic Peptide
YEETRGRIS

SEQ ID NO: 253 - protein, 9 aa, Artificial Sequence
  Synthetic Peptide
FDKLKEFFQ

SEQ ID NO: 254 - protein, 9 aa, Artificial Sequence
  Synthetic Peptide
VSTPTLVEV

SEQ ID NO: 255 - protein, 9 aa, Artificial Sequence
  Synthetic Peptide
VNDLFERLK

SEQ ID NO: 256 - protein, 9 aa, Artificial Sequence
  Synthetic Peptide
LPGELAKHR

SEQ ID NO: 257 - protein, 9 aa, Artificial Sequence
  Synthetic Peptide
VNDIFERIK

SEQ ID NO: 258 - protein, 9 aa, Artificial Sequence
  Synthetic Peptide
FSQYLQQCP

SEQ ID NO: 259 - protein, 9 aa, Artificial Sequence
  Synthetic Peptide
ITKAKDAFL

SEQ ID NO: 260 - protein, 9 aa, Artificial Sequence
  Synthetic Peptide
LGEYGFQNA

SEQ ID NO: 261 - protein, 9 aa, Artificial Sequence
  Synthetic Peptide
LCDEFKAKL

SEQ ID NO: 262 - protein, 9 aa, Artificial Sequence
  Synthetic Peptide
VDKCCAADD

SEQ ID NO: 263 - protein, 9 aa, Artificial Sequence
  Synthetic Peptide
VNDIFERIR

SEQ ID NO: 264 - protein, 9 aa, Artificial Sequence
  Synthetic Peptide
ISGLIYEET

SEQ ID NO: 265 - protein, 9 aa, Artificial Sequence
  Synthetic Peptide
LAKHRNDEE

SEQ ID NO: 266 - protein, 19 aa, Artificial Sequence
  Synthetic Peptide
SGGGSKMRMATPLLMQALY

SEQ ID NO: 267 - protein, 15 aa, Artificial Sequence
  Synthetic Peptide
SKMRMATPLLMQALY

SEQ ID NO: 268 - protein, 21 aa, Artificial Sequence
  Synthetic Peptide
SGGGANSGFRIMAVLASGGQY

SEQ ID NO: 269 - protein, 17 aa, Artificial Sequence
  Synthetic Peptide
ANSGFRIMAVLASGGQY

SEQ ID NO: 270 - protein, 18 aa, Artificial Sequence
  Synthetic Peptide
SGGGKALVQNDTLLQVKG